(19) United States
(12) Patent Application Publication
(10) Pub. No.: US 2009/0123516 A1
(43) Pub. Date: May 14, 2009

(54) DRUG DELIVERY FROM IMPLANTS USING SELF-ASSEMBLED MONOLAYERS-THERAPEUTIC SAMS

(75) Inventors: C. Mauli Agrawal, San Antonio, TX (US); David Johnson, Bulverde, TX (US); Gopinath Mani, San Antonio, TX (US); Anil Mahapatro, San Antonio, TX (US); Marc Feldman, San Antonio, TX (US); Devang Patel, San Antonio, TX (US); Arturo Ayon, San Antonio, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/997,092

(22) PCT Filed: Aug. 8, 2006
(86) PCT No.: PCT/US06/30818

§ 371 Date: Jun. 26, 2008

Related U.S. Application Data
(60) Provisional application No. 60/706,266, filed on Aug. 8, 2005.

(51) Int. Cl. A61F 2/82; A61F 2/02; A61K 38/43; A61K 39/395; A61K 31/7088; A61K 38/02; A61K 31/485; A61P 35/00; A61P 9/00; A61P 31/00; A61P 31/04; A61P 31/10; A61P 19/02

(52) U.S. Cl. 424/423; 424/422; 514/2; 424/94.1; 424/178.1; 514/44; 514/282

(57) ABSTRACT

Disclosed are medical devices comprising one or more surfaces, one or more SAM molecules attached to the one or more surfaces of the medical device, and one or more therapeutic agents attached to the one or more self-assembled monolayer molecules. Also disclosed are medical devices comprising one or more surfaces, one or more self-assembled monolayer molecules attached to the one or more surfaces of the medical device, one or more linkers comprising a first functional group and a second functional group, the first functional group attached to the self-assembled monolayer molecule and a therapeutic agent attached to the second functional group. The therapeutic agent may be attached to the SAM molecule via a linker. The present invention also concerns methods of administering a therapeutic agent to a subject, comprising contacting the subject with one of the medical devices set forth herein.

This patent application claims the benefit of priority to U.S. Provisional Patent Application Ser. No. 60/706,266, filed Aug. 8, 2005, which has the same title and inventors as the present application, and is hereby incorporated by reference in its entirety.

### BACKGROUND OF THE INVENTION

1. Field of the Invention

The present invention relates generally to the field of self-assembled monolayers (SAMs), medical devices, and pharmacotherapeutics. More particularly, it concerns medical devices comprising one or more surfaces, one or more SAM molecules attached to the one or more surfaces of the medical device, and one or more therapeutic agents attached to the one or more self-assembled monolayer molecules. The therapeutic agents may be attached to the SAM molecules via a linker. The present invention also concerns methods of administering a therapeutic agent to a subject, comprising contacting the subject with one of the medical devices set forth herein.

2. Description of Related Art

Stents are small, expandable, metal devices inserted by a catheter into a narrowed artery of a patient following completion of angioplasty. Stents are left in place to prevent restenosis of the artery. In recent years, coronary stenting has emerged as a significant breakthrough in the field of interventional cardiology. There has been an explosive use of these device in coronary interventional cases, in as much as 70% to 80% in some of the high volume centers.

Bare metal coronary stents have reduced angiographic restenosis rate (from 30-40% with percutaneous transluminal coronary angioplasty (PTCA)) to 20-30% (Serruys et al., 1994; Fischman et al., 1994), by providing scaffolding that eliminates vessel recoil and negative remodeling. However, in-stent restenosis because of neo-intima formation remains a significant problem (Hoffman et al, 1996). Neo-intima consists mainly of smooth muscle cells and their secreted collagen matrix (Komatsu et al, 1998). Vessel injury during stent expansion triggers a release of various cytokines, which act as mediators of smooth muscle cell migration and proliferation (Kornowski et al., 1998; Rectenwald et al., 2000).

Restenosis and the need for repeat procedures limits the long term benefit of coronary stents, especially in certain subgroups. In particular, longer lesions and smaller vessels have progressively higher rates of restenosis (Al Suwaidi et al., 2001; Cura et al., 2003; Serruys et al., 2002). Diabetic patients have higher rates of restenosis, even after correcting for their smaller coronary vessels and diffuse nature of their coronary artery disease (Abizaid et al., 2001).

Since the architecture and mechanical characteristics of stents are nearly optimized, the only area remaining open for restenosis prevention is in-situ treatment. It is hypothesized that if smooth muscle proliferation after stenting could be prevented, this would eliminate restenosis.

Pharmacological therapy has not been successful in preventing restenosis. One of the main reasons for the failure of systemic pharmacological therapy is the non-availability of the required dose at the site of injury (Bonan et al., 1991). Earlier approaches for delivering drugs locally by using catheters were not successful due to rapid washout of the drugs in the blood stream. Currently, two distinct approaches have been taken. Since radiation inhibits cell proliferation, various catheter-based antra-coronary brachytherapy systems have been developed. Although brachytherapy is available for treatment of in-stent restenosis (secondary prevention), it is not recommended for stenting of de-novo lesions (primary prevention) because of a higher risk of sub-acute stent thrombosis (Nguyen-Ho et al., 2002).

This paved the way for development of stent based local drug delivery. Although numerous drug candidates have been identified because of positive outcomes in cultured smooth muscle cells and subsequently animal models, most of these agents have not shown benefit in humans. So far, two agents have shown the ability to significantly reduce restenosis rate in clinical trials: the Sirolimus (rapamycin) eluting Cypher™ stent (Moses et al., 2003, and Morice et al, 2002) and the Paclitaxel eluting Taxus™ stent (Colombo et al, 2003, and Ellis 2003), although reports on adverse inflammatory responses are emerging.

The development of drug eluting stents must overcome two major hurdles. Firstly, there is the need to develop a mechanism by which the agent can be loaded onto a stainless steel stent surface. Various strategies have been attempted, but the most well-known has been to coat the stent with a polymer (biodegradable or non-biodegradable). Certain drug-eluting stents use a non-biodegradable polymer that incorporate a biologically active material, such as thrombolytic agents (see U.S. Pat. No. 6,099,562, U.S. Pat. No. 5,879,697, U.S. Pat. No. 5,092,877, and U.S. Pat. No. 5,304,121). Such coatings have been applied to the surface of a medical device by various methods, e.g., spray coating and dip coating.

A major drawback is that all polymers (particularly biodegradable polymers) induce an inflammatory reaction to some extent, which contributes to restenosis (van der Glessen et al, 1996). Current evidence suggests that adverse reactions are caused by polymers. Several cases have been reported recently about the hypersensitivity reactions to drug eluting stents (Virmani et al., 2004a; Virmani et al., 2004b; Virmani et al., 2004c; Nebeker et al., 2006). In an pathological study of stent related hypersensitivity reactions, it was noted that the polymer fragments were detaching from the stent struts and were surrounded by giant cells and eosinophils (Virmani et al., 2004a). Stent-induced inflammatory reactions predominantly consisted of T lymphocytes and eosinophils with extensive inflammation of the arterial wall (Virmani et al., 2004a; Virmani et al, 2004b). Already, the FDA has posted a cautionary view about the adverse and hypersensitive reactions following the deployment of sirolimus-eluting CYPHER stents (Virmani et al., 2004; McFadden et al., 2004; Lakovou et al., 2005).

A second major hurdle has been to control drug delivery. The biology of restenosis is such that the agents need to be present for a period of 2-4 weeks after stent delivery to effectively inhibit neo-intima formation. Resolving this by loading the stent with more agents leads to a large and toxic quantity of agent being delivered to the vessel wall within hours of stent deployment (Farb et al., 2001). A mechanism by which agents can be delivered over a 2-4 week period, while avoiding local toxicity would be ideal.

There is a concern that once the drug is depleted, polymer-induced inflammation will no longer be suppressed. This may lead to late neo-intimal proliferation or restenosis—so called catch-up effect. While the reductions in restenosis (27-36% for bare metal stents to 5-9% for drug-eluting stents) reported in the clinical trials have been impressive, restenosis rates in certain subgroups remain high (10-15% for small vessels, 10-15% for long lesions and 6-16% for diabetics) (Moses et al., 2003, and Ellis, 2003). It is likely that higher restenosis rates will be found, as the use of these stents is expanded beyond the initial narrow inclusion criteria (simple lesions) applied in clinical trials, to more complex lesions (ostial, bifurcation and long lesions, small vessels, vein grafts and chronic total occlusions) and varied clinical scenarios encountered in clinical practice. Also since the drugs and the polymer need to be dissolved in a common solvent and then coated onto the stent, this restricts the number of drugs available for polymer based drug delivery. For example polymers are unable to carry proteins or genes as anti-restenotic agents.

Self-assembled monolayers (SAMs) are formed from molecules that have a chemical group which binds to a surface strongly, and a portion of the molecules which will bind to neighboring molecules in a monolayer film. The utility of SAMs is evident from their name: the monolayer is formed by virtue of the chemical structure of its constituent molecules. Despite extensive literature concerning SAMs on many metals (e.g., Schreiber, 2000; Ulman, 1996; Allara et al., 1991; Tao, 1993; Schlotter et al., 1986; Chau and Porter, 1990; Folkers et al., 1995; Lin et al., 2002, each of which is herein specifically incorporated by reference), SAMs formed on other metals such as titanium (Hofer et al., 2001; Tosatti et al., 2002; Zwahlen et al., 2002; Chen et al., 2001) and 316L stainless steel (Meth and Sukenik, 2003; Shustak et al., 2004; Ruan et al., 2002) are considerably less well-studied, SAMs on gold being the most studied metal (Schreiber, 2000; Ulman, 1996).

Surface modification of SAMs have been carried out to immobilize peptides, proteins and other biomolecules to the surface to prepare the complex surface required for well defined biological experiments (Castner and Ratner, 2002). For example, polylysine was covalently attached via amide bonds to an alkanethiol SAM on gold, for applications in developing biosensors (Frey and Corn, 1996). A mixture of SAMs was synthesized and derivatives of polyethylene glycol was covalently attached to form SAMs that resist adsorption of proteins (Chapman et al., 2000). Limited information pertaining to application of SAMS on a gold surface of a medical device (see U.S. Patent Application Pub. No. 20040037836) or gold/silver surface (U.S. Pat. No. 6,617,027).

316L, a medical grade stainless steel (SS), used extensively for the manufacturing of implantable medical devices (Shustak et al., 2004), is currently used in cardiovascular implant applications such as coronary stents. Attachment of therapeutic drugs to SAMs after their assembly on 316L SS could possibly serve as a localized drug delivery system, which, if used in coronary stents, could reduce arterial restenosis. It could also minimize or eliminate some of the problems with current technologies such as allergic reactions to the polymers used on stents for drug delivery.

A variety of terminal functional groups and their chemical transformations on SAMs after their assembly have been examined (see, e.g., Sagiv et al., 1980; Duevel and Corn, 1992). These studies have shown that many organic reactions that work well in solution are difficult to apply at surfaces because of steric hindrance. In such a hindered environment, backside reactions (e.g., S_{N}2 reaction) and reactions with large transition state (e.g., esterification, saponification, Diels-Alder reaction and others) often proceed slowly (Yan et al., 2004). Use of enzymes in organic synthesis and polymer science is well-established, and has been discussed elsewhere within comprehensive reviews (Roberts, 2001). Recent advances in nonaqueous enzymology have significantly expanded the potential conditions under which these reactions can be performed. Use of an enzyme for surface modification of SAMs on metal surfaces would offer distinct advantages.

There are numerous reports of hydrolysis of lipid monolayers using different lipases (Tanaka and Yu, 2002; Laboda et al., 1988). Relatively few reports demonstrate lipase catalyzed esterification synthesis on air/water monolayers (Singh et al., 1993; Singh et al., 1994). Specifically, Singh, et al. (1993) have reported use of lipase lipozyme for the synthesis of glycerol and fatty acid on stearic acid monolayers. Singh, et al (1994) have also reported lipase catalyzed synthesis esterification of oleic acid with glycerol in monolayers. Turner, et al. (1996) have reported the hydrolysis of a phospholipid film which was covalently attached via chemical methods to a silica surface. However, there are no published reports of lipase-catalyzed esterification of therapeutic drugs to functional SAMS has not been demonstrated.

Thus, there is the need for novel forms of stents and other medical devices that can be designed to deliver therapeutic agents in a controlled fashion to a target tissue without eliciting an adverse response.

### SUMMARY OF THE INVENTION

The inventors have discovered certain novel medical devices that incorporate a therapeutic agent through the use of self-assembled monolayer (SAM) molecules. For example, the medical device can be coated with a SAM, wherein one or more therapeutic agents are attached to the SAM via a linker interposed between SAM molecules and therapeutic agents. Also discovered are certain novel methods of delivery of a therapeutic agent to a subject that involve contacting the subject with one of the novel medical devices set forth herein.

Within the SAM, individually small, but cumulatively large, forces drive the molecules into a self-assembly process, forming a molecular coating with precise and reproducible physical properties. In some embodiments, only the SAM molecules will be present at the implant surface, and only the therapeutic agent will be present at the implant-tissue interface. This level of precision creates opportunities for highly consistent dose delivery of therapeutic agents. This technology represents a dramatic improvement over polymer coatings because the SAMs form a molecular layer that is integrated on part or all of the implant surface. In some embodiments, this results in a coating that will expand or contract uniformly with the implant while maintaining structural integrity and chemical composition.

In this regard, certain embodiments of the present invention generally pertain to a medical device comprising one or more surfaces, one or more SAM molecules attached to the one or more surfaces of the medical device, and one or more therapeutic agents attached to the one or more SAM molecules. A “medical device” is defined herein to refer to an instrument, apparatus, implement, machine, contrivance, implant, in vitro reagent, or other similar or related article, including a component part, or accessory which is: (a) intended for use in the diagnosis of disease or other conditions, or in the cure, mitigation, treatment, or prevention of disease, in a subject, or (b) intended to affect the structure or any function of the body of a subject. A “self-assembled monolayer molecule” is defined herein to refer to a molecule that has one or more chemical groups which attach to a surface strongly, wherein a portion of the molecule will bind to one or more neighboring self-assembled monolayer molecules in a monolayer film, or “self-assembled monolayer” (SAM). Additional information pertaining to medical devices, SAM molecules, and SAMs is addressed in greater detail in the specification below.

Other embodiments of the present invention generally pertain to a medical device comprising one or more surfaces, one or more self-assembled monolayer molecules attached to the one or more surfaces of the medical device, one or more linkers comprising a first functional group and a second functional group, the first functional group attached to the self-assembled monolayer molecules and a therapeutic agent attached to the second functional group. A “linker” is defined herein to refer to a molecule comprising two or more functional groups, wherein one of the functional groups is capable of forming an attachment to a SAM molecule, and wherein a second functional group is capable of forming an attachment to a therapeutic agent. Linkers are discussed in greater detail in the specification below.

The medical device may include more than one self-assembled monolayer molecules forming one or more self-assembled monolayers (SAM) coating a portion or all of one or more surface of the medical device. SAM molecules are defined and discussed in the specification below.

The medical devices set forth herein may be comprised of any material known to those of ordinary skill in the art. Examples include stainless steel, titanium, tantalum, cobalt, chromium, gold, silver, platinum, a polymer, a polymer derivative, a copolymer, a multi-component copolymer, glass, pyrolytic carbon, alumina, zirconia, titania, graphite, and a ceramic. In some embodiments, the medical device is comprised of an alloy of two or more metals selected from the group consisting of stainless steel, titanium, tantalum, cobalt, chromium, gold, silver, and platinum. In certain particular embodiments, the alloy is nitinol. In other particular embodiments, the material is stainless steel, such as 316L stainless steel.

The medical device may also be comprised of one or more polymers selected from the group consisting of poly(ethylene glycol), poly(caprolactone), poly(hydroxyethyl methacrylate), poly(lactic acid), poly(ethylene), poly(glycolic acid), poly(styrene), a poly (anhydride), a poly(urethane), a poly(carbamate), a poly(ester), and a derivative thereof. In some embodiments, the polymer is further defined as a terpolymer or a polymer blend.

In certain embodiments of the present invention, a SAM is attached to one surface of the medical device. Alternatively, a SAM may be attached to more than one surface of the medical device. As set forth in the specification below, the surface can be any surface, such as a surface of the medical device that will be in contact with tissue following implantation of the medical device in a subject. In other embodiments, a SAM is attached to a portion of one or more surface of the medical device.

Any method known to those of ordinary skill in the art can be used to attach the one or more SAM molecule to the one or more surface of the medical device. For example, the attachment may be via one or more moiety selected from the group consisting of a thiol, a disulfide, a dithioic acid, a dithiocarbamate, a silane, a chlorosilane, a dichlorosilane, a trichlorosilane, an alkoxysilane, a dialkoxysilane, a trialkoxysilane, a hydroxyamic acid, a phosphate, a phosphonic acid, a carboxylic acid, a hydroxamic acid, an alcohol, an amine, a sulfate, a sulfonate, and a sulfinate. In some particular embodiments, the one or more self-assembled monolayer molecule is attached to the one or more surface via a thiol moiety. In other particular embodiments, the one or more self-assembled monolayer molecules are attached to the one or more surfaces via a silane or silane derivative. In further particular embodiments, the one or more self-assembled monolayer molecules are attached to the one or more surfaces via a phosphonate or phosphate.

In some embodiments, the one or more SAM molecules are comprised of carbon atoms. There may be any number of carbon atoms in each SAM molecule. In some embodiments, for example, the one or more self-assembled monolayer molecules are comprised of six to thirty-nine carbon atoms. In more particular embodiments, the one or more self-assembled monolayer molecules are comprised of eight, nine, ten, eleven or twelve carbon atoms.

In other embodiments of the present invention, the medical device further includes a polymer or a peptide attached to the one or more of the self-assembled monolayer molecules. Addition of a polymer may facilitate controlled release of the therapeutic agent. Polymers are discussed in greater detail in the specification below. In certain preferred embodiments, the polymer is poly(ethylene glycol). In some embodiments, the peptide is a cellular adhesion peptide.

The medical device may include more than one type of self-assembled monolayer molecules. These embodiments may further include a polymer or peptide attached to the SAM molecule. In certain preferred embodiments, the polymer is poly(ethylene glycol). In some embodiments, the peptide is a cellular adhesion peptide. As discussed above, the SAM molecules can be attached to the one or more surfaces via any mechanism known to those of ordinary skill in the art, such as via a thiol, a disulfide, a dithioic acid, a dithiocarbamate, a silane, a chlorosilane, a dichlorosilane, a trichlorosilane, an alkoxysilane, a dialkoxysilane, a trialkoxysilane, a hydroxyamic acid, a phosphate, a phosphonic acid, a carboxylic acid, a hydroxamic acid, an alcohol, an amine, a sulfate, a sulfonate, or a sulfinate moiety.

In those embodiments comprising a linker, any linker known to those of ordinary skill in the art is contemplated. Exemplary linkers include of polyethylene glycol, a dendrimer, a molecule comprising a tert-butyl protecting group, a molecule comprising an isobutylene oxide connection, an amino benzyl alcohol, a hydroxy benzyl alcohol connection, an aminobenzene dimethanol, an aminobenzene trimethanol, a hydroxybenzene dimethanol, a hydroxybenzene trimethanol, a vinyl sulfoxide, a substituted vinyl sulfoxide, a substituted methoxymethyl connection, a substituted vinyl ether connection, a carbonate connection, an ester connection, an anhydride connection, a substituted carbamic anhydride connection, a carbonic anhydride connection, an substituted urea connection, a substituted urethane connection, a substituted guanidine connection, a ether connection, a mercaptan connection, a sulfoxide connection, a sulfinate connection, a sulfonate connection, a sulfenate connection, a nitronate connection, a sulfite connection, a sulfate connection, a phosphate connection, a phosphonate connection, a phosphine connection, a silane connection, a silicate connection, a disulfide connection, a peroxide connection, an alkane connection, an alkene connection, an alkyne connection, an iodonium connection, an amino connection, a substituted allyl ether connection, a substituted benzyl ether connection and an imine connection. In certain particular embodiments, the linker is a dendrimer or dendritic structure. Dendrimers and dendritic structures are discussed in greater detail in the specification below. The dendritic structure or dendrimer may be capable of disassembly, self-immolation, release by dendritic amplification, or cascade-release.

The first and second functional groups of the linker can be any type of functional group known to those of ordinary skill in the art. Exemplary functional groups include a hydroxyl, a carboxyl, an amino, a phosphate, a phosphonate, a sulfate, a sulfite, a sulfenate, a sulfinate, a sulfonate, a sulfoxide, a sulfone, an amide, an ester, an ketone, an aldehyde, a nitrile, an alkene, an alkyne, an ether, a thiol, a hydroxyamic acid, a silane, a silicate, a carbamodithionate, a dithionate, a mercaptan, a disulfide, a peroxide or a nitronate.

The attachment between the linker and one or more SAM molecule may be covalent or non-covalent. Similarly, the attachment between the linker and the therapeutic agent may be covalent or non-covalent.

Any therapeutic agent is contemplated by the present invention. Therapeutic agents are discussed in greater detail in the specification below. Examples of types of therapeutic agents include a small molecule, a peptide, a polypeptide, a protein, an enzyme, an antibody, a DNA molecule, and an RNA molecule. Exemplary therapeutic agents include an anticancer agent, a hormone, an anesthetic agent, a vasodilator, an anticoagulant, an anti-inflammatory agent, a steroid, an antibiotic, an antiseptic, an antifungal, an opiate, an analgesic, an antiproliferative agent, or an anti-platelet agent. In particular embodiments, the therapeutic agent is rapamycin, sirolimus, a taxol, everolimus, tacrolimus, dexamethasone, prednisolone, morphine, or fentanyl. The taxol can be any taxol, such as paclitaxel. Further, the medical device can include more than one type of therapeutic agent.

The medical device can be any type of medical device known to those of ordinary skill in the art. For example, the medical device may further defined as a medical device suitable for implantation in a subject. Exemplary medical devices include a stent, a valve, a metal plate, a musculoskeletal fixation system, a pin, an artificial joint, a dental implant, a temporal mandibular joint, an ocular implant, a neural implant, an artificial heart, and an artificial organ, and an implant in contact with body fluids. In certain particular embodiments of the present invention, the medical device is a stent, such as a coronary stent. Other types of stents include an arterial stent, a GI stent, a pulmonary stent, a vascular stent, and a ureteral stent. In other embodiments, the medical device is further defined as a medical device suitable for application to a surface of a subject. The surface of the subject may be any surface, such as a skin surface, a mucosal surface, a wound surface, a surface of a hollow viscus, or a tumor surface.

In further embodiments of the present invention, the medical device comprises one or more openings in one or more surfaces of the medical device. The openings can be of any size or shape. For example, surface can be further defined as a nanoporous surface. Thus, the medical devices set forth herein can comprise one or more nanoporous surfaces. A “nanoporous surface” to refer to a surface that is comprised of one or more openings with a diameter in the nanometer scale. In certain particular embodiments, the body of the medical device is a nanoporous body. A “nanoporous body” is a body of a medical device that is comprised of one or more openings with a diameter in the nanometer scale, ranging from 0.1 nm to 100 nm. The nanoporous body comprises a substance with a bicontinuous, partially bicontinuous or non-bicontinuous material in which one of the phases of the body comprises the material from which the body is built and the other phase is empty void space, air, or filled void space. In certain embodiments set forth herein, a SAM molecule is attached or a SAM coats the surface comprising one or more openings. Such a coating may facilitate increased surface area of the medical device, and thus increased capacity for attachment of therapeutic agents to the medical device.

As discussed in greater detail in the specification below, in certain particular embodiments set forth herein, the medical device is capable of releasing the therapeutic agent in a subject following contact of the medical device with a subject. Release can be by any mechanism known to those of ordinary skill in the art. For example, the therapeutic agent may be released by hydrolysis, oxidation, reduction, cycloaddition, retro-cycloaddition, ring-closure, decomposition, disproportionation, electrophilic cleavage, nucleophilic cleavage, aminolysis, alcoholysis, elimination, and solvolysis, acid catalysis, biocatalysis, or base catalysis following implantation of the medical device in a subject.

The present invention also generally pertains to use of any of the medical devices set forth above for treating a disease in a subject. In certain embodiments, the subject is a human. For example, the human may be a patient in need of the therapeutic agent or treatment or prevention of a disease. For example, the disease may be a cardiovascular disease, hyperproliferative disease, coronary artery disease, valvular heart disease, heart failure, peripheral vascular disease, ureteral obstruction, bile duct obstruction, bronchial or tracheal obstruction, arthritis, degenerative joint disease, a bone fracture, arthritis, degenerative joint disease, cancer, or a cardiac arrhymthia. In some embodiments, the patient is further defined as a patient in need of surgical therapy with implantation or application of a medical device for treatment or prevention of cardiovascular disease, hyperproliferative disease, coronary artery disease, valvular heart disease, heart failure, peripheral vascular disease, ureteral obstruction, bile duct obstruction, bronchial or tracheal obstruction, arthritis, degenerative joint disease, a bone fracture, arthritis, degenerative joint disease, cancer, or a cardiac arrhymthia. The use may further be defined as comprising administering one or more secondary forms of therapy.

The present invention is also directed to methods of administering a therapeutic agent to a subject, comprising contacting the subject with a medical device comprising one or more surface, one or more SAM molecule attached to the one or more surface of the medical device, and one or more therapeutic agent attached to the one or more SAM molecule. The present invention is also directed to methods of administering a therapeutic agent to a subject, comprising contacting the subject with a medical device comprising one or more surfaces, one or more self-assembled monolayer molecules attached to the one or more surfaces of the medical device, one or more linkers comprising a first functional group and a second functional group, the first functional group attached to a self-assembled monolayer molecule and a therapeutic agent attached to the second functional group. The medical device can be any of those medical devices discussed above and elsewhere in this specification.

In some embodiments of the present invention, the method further includes release of the therapeutic agent following contact of the medical device with the subject. The subject can be any subject, such as an avian species or a mammal. The mammal can be any mammal, such as a human or a laboratory animal. In certain particular embodiments, the mammal is a patient in need of the therapeutic agent or treatment or prevention of a disease.

The disease can be any disease or health-related condition. Exemplary diseases include cardiovascular disease, hyperproliferative disease, coronary artery disease, valvular heart disease, heart failure, peripheral vascular disease, ureteral obstruction, bile duct obstruction, bronchial or tracheal obstruction, arthritis, degenerative joint disease, a bone fracture, arthritis, degenerative joint disease, cancer, a cardiac arrhymthia, or sudden death as a result of cardiovascular disease. The patient may be a patient in need of surgical therapy with implantation or application of a medical device for treatment or prevention of cardiovascular disease, hyperproliferative disease, coronary artery disease, valvular heart disease, heart failure, peripheral vascular disease, ureteral obstruction, bile duct obstruction, bronchial or tracheal obstruction, arthritis, degenerative joint disease, a bone fracture, arthritis, degenerative joint disease, cancer, a cardiac arrhymthia, or sudden cardiac death. The therapeutic agent can be any of those agents discussed above and elsewhere in this specification.

Some embodiments of the present invention further comprise identifying a subject in need of the therapeutic agent. Identifying a subject in need can include any method known to those of ordinary skill in the art. Examples of such methods include identification of subjects by medical history, identification of subjects based on their physical examination by a physician, identification of subjects that have undergone certain medical tests and procedures, and so forth.

Other embodiments of the methods set forth herein pertain to methods of preventing a disease in a subject. “Preventing” refers to the halting of onset of a disease. The disease can be any disease or health-related condition. Examples include those diseases set forth above. In certain embodiments, the methods further concern identifying a patient in need of preventive therapy. Identification of a patient in need of preventive therapy can include any method known to those of ordinary skill in the art. Exemplary methods include identification of subjects at risk based on family history of a particular disease or other clinical criteria familiar to those of ordinary skill in the art.

The one or more secondary form of therapy can be any secondary form of therapy known to those of ordinary skill in the art. Examples, discussed in more detail in the specification below, include secondary pharmacotherapy, secondary surgical therapy, radiation therapy, chemotherapy, gene therapy, and/or immunotherapy.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic diagram of self-assembled monolayer molecules on a metal surface.

FIG. 2. XPS spectra of self-assembled monolayers in the S region for HO-SAMS.

FIG. 3. ¹H NMR spectra of aspirin attached NH₄-12-HDDA ester.

FIG. 4. XPS C (1s) spectra of NH₄-12-HDDA and Asp-NH₄-12-HDDA, self-assembled onto Ti-6Al-4V by AT-AS procedure.

FIG. 5. XPS O (1s) spectra of NH₄-12-HDDA and Asp-NH₄-12-HDDA, self-assembled onto Ti-6Al-4V by AT-AS procedure.

FIG. 6. XPS C (1s) spectra of NH₄-12-HDDA and Asp-NH₄-12-HDDA, self-assembled onto Ti-6Al-4V by AS-AT procedure.

FIG. 7. XPS O (1s) spectra of NH₄-12-HDDA and Asp-NH₄-12-HDDA, self-assembled onto Ti-6Al-4V by AS-AT procedure.

FIG. 8. Comparison of XPS spectra of SAMs in the C region [Peak identification: (a) C—C, 284.7 eV; (b) C—O; 286.5 eV; (c) C═O; 288 eV]

FIG. 9. Comparison of XPS spectra of SAMs in the O region [Peak identification: (a) metal oxide; 530.1 eV; (b) O—H, 531.8 eV, (c) C—O—C═O; 288 eV].

FIG. 10. Effect of surface modification on contact angle of 316L SS. A—As received SS; B—after chemical treatment; C—after plasma treatment; D—OH SAM; E—OH SAM+ibuprofen; F—COOH SAM; G—COOH SAM+perphenazine.

FIG. 11. XPS spectra of S 2p for functional SAMs on 316L SS.

FIG. 12. FTIR spectra of —COOH terminated SAM on 316L for before and after esterification via lipase catalysis [Control 1: control reaction with drug and without Novozyme-435, Control 2: control reaction with Novozyme-435 but without drug].

FIG. 13. Scheme showing lipase-catalyzed esterification of —OH SAMs with ibuprofen.

FIG. 14A, 14B. FIG. 14A—Lipase catalyzed esterification of —COOH SAMs with perphenazine; FIG. 14B—XPS spectra of the C (1s) region of functional SAMs on 316L SS for (a) ibuprofen and (b) perphenazine before and after esterification via lipase catalysis. [Control 1: control reaction with drug and without Novozyme-435, Control 2: control reaction with Novozyme-435 but without drug]

FIG. 15. XPS spectra of the C (1s) region of functional SAMs on 316L SS for (a) Ibuprofen and (b) perphenazine before and after esterification via lipase catalysis.

FIG. 16. High-resolution XPS spectra of the C 1s region for the HS(CH₂)₁₁OH SAMs on gold substrates.

FIG. 17. High-resolution XPS spectra of the O 1s region for the HS(CH₂)₁₁OH SAMs on gold substrates.

FIG. 18. Formation of SAMs on titanium surfaces: Contact angle measurements for the optimized and SAMs formed titanium surfaces.

FIG. 19. Formation of T-SAMs: schematic representation of drug attachment chemical reactions.

FIG. 20. High-resolution XPS spectra of C 1s region for the T-SAMs_{(Asprin)} on gold substrates.

FIG. 21. High-resolution XPS spectra of O 1s region for the T-SAMs_{(Aspirin)} on gold substrates.

FIG. 22. High-resolution XPS spectra of F 1s region for the T-SAMs_{(Diflunisal)} on gold substrates.

FIG. 23. High-resolution XPS spectra of F 1s region for the T-SAMs_{(Flufenamic acid)} on gold substrates.

FIG. 24. High-resolution XPS spectra of N 1s region for the T-SAMs_{(Flufenamic acid)} on gold substrates.

FIG. 25A, 25B. FIG. 25A-Chemical structure of diflunisal (left) and flufenamic acid (right); FIG. 25B-high-resolution XPS spectra of C 1s region for the T-SAMs_{(Diflunisal)} on gold substrates.

FIG. 26. High-resolution XPS spectra of C 1s region for the T-SAMs_{(Flufenamic acid)} on gold substrates.

FIG. 27. High-resolution XPS spectra of O 1s region for the T-SAMs_{(Diflunisal)} on gold substrates.

FIG. 28. High-resolution XPS spectra of O 1s region for the T-SAMs_{(Flufenamic acid)} on gold substrates.

FIG. 29. Calibration plots for the determination of aspirin by reverse-phase HPLC.

FIG. 30. Cumulative in vitro drug release profiles for T-SAMs_{(Aspirin)} on gold substrates.

FIG. 31. Atomic concentration (%) of the ester components in the XPS C 1s spectra of T-SAMs_{(Aspirin)} formed gold substrates and aspirin eluted samples at different time points.

FIG. 32. Atomic concentration (%) of the ester components in the XPS O 1s spectra of T-SAMs_{(Aspirin)} formed gold substrates and aspirin eluted samples at different time points.

FIG. 33. Atomic concentration (%) of the thiol (BE=162.2±0.4 eV) and oxidized thiol (BE=169.3±0.6 eV) components in the XPS S 2p spectra.

FIG. 34. High-resolution XPS spectra of S 2p region for the SAMS, T-SAMs_{(Aspirin)}, and aspirin eluted samples at 30 days on gold substrates.

FIG. 35. Stability of phosphate SAMs on titanium surfaces: Contact angle measurements of phosphate SAMs before and after saline solution treatment.

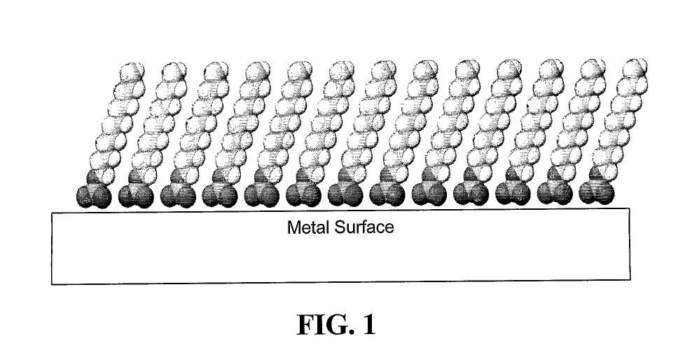

### DESCRIPTION OF ILLUSTRATIVE EMBODIMENTS

The inventors have discovered certain novel medical devices that incorporate a therapeutic agent through the use of self-assembled monolayer (SAM) molecules. For example, in some embodiments, one or more therapeutic agents is attached to the SAM via a linker. Novel methods of delivery of therapeutic agents using the aforementioned medical devices are also set forth herein.

The technology set forth herein can be used to effectively release therapeutic agents from medical devices, such as intravascular stents. SAMs comprising therapeutic agents, which will be only a few nanometers (<20 nm) in thickness, will afford several advantages over current systems: (a) the properties of the T-SAMs can be designed at the molecular level; (b) the self-assembly process will greatly simplify manufacturing of therapeutic implants; (c) the base SAM may be made biologically inert; (d) the nanometer scale of the SAMs will deform with the implant without damage to the coating; (e) the release rate of the therapeutic agent can be highly reproducible; and (f) the amount of therapeutic agent loaded will be highly reproducible.

### A. SELF-ASSEMBLED MONOLAYER MOLECULES AND SELF-ASSEMBLED MONOLAYERS

1. Definitions

A “self-assembled monolayer molecule” is defined herein to refer to a molecule that has one or more chemical groups which attach to a surface strongly, wherein a portion of the molecule will bind to one or more neighboring self-assembled monolayer molecules in a monolayer film, or “self-assembled monolayer” (SAM). Within the SAM, individually small, but cumulatively large, forces drive the SAM molecules into a self-assembly process, forming a molecular coating (i.e., SAM) with precise and reproducible physical properties. In essence, each SAM molecule is bound to the surface, and to the film of neighboring molecules. The utility of SAMs is evident from their name: the monolayer is spontaneously formed by virtue of the chemical structure of its constituent molecules.

A “monolayer film,” in the context of the present invention, is defined herein to refer to a layer that is the thickness of one SAM molecule that is attached to a surface.

A “surface” is defined herein to refer to a superficial, topmost, outer, or external aspect of an object. In the context of the present invention, the object is a medical device. Medical devices, and materials that comprise medical devices, as discussed in greater detail in the specification below.

The chemical group which attaches to a surface strongly can be any chemical group known to those of ordinary skill in the art which is able to attach to a surface. The attachment can be covalent (such as SAMS based on ionic or polar chemical functional groups such as, but not limited to, phosphonates, phosphates, carboxylates, or their corresponding acids). The surface can be composed of any agent or combination of agents, so long as the SAM molecule is able to attach to the surface. Surfaces of medical devices are discussed in greater detail in the specification below.

Exemplary chemical groups for attachment to a surface include the following: a thiol, a disulfide, a dithioic acid, a dithiocarbamate, a silane, a chlorosilane, a dichlorosilane, a trichlorosilane, an alkoxysilane, a dialkoxysilane, a trialkoxysilane, a methyldichlorosilane, a dimethyl chlorosilane, other silane derivatives, a hydroxyamic acid, a phosphate, a phosphonic acid, a carboxylic acid, a hydroxamic acid, an alcohol, an amine, a sulfate, a sulfonate, and a sulfinate. One of ordinary skill in the art would be familiar with these and other chemical groups that are able to form an attachment to the surface.

In the context of the present invention, the SAM molecule includes one or more additional chemical groups that is able to attach to a first functional group of a linker. Linkers are defined and discussed in greater detail in the specification below. The additional chemical group can be any chemical group known to those of ordinary skill in the art that has the ability to form an attachment to a linker. Examples of such additional chemical groups include a hydroxyl, a carboxyl, an amino, a phosphate, a phosphonate, a sulfate, a sulfite, a sulfenate, a sulfinate, a sulfonate, a sulfoxide, a sulfone, an amide, an ester, an ketone, an aldehyde, a nitrile, an alkene, an alkyne, an ether, a thiol, a hydroxyamic acid, a silane, a silicate, a carbamodithionate, a dithionate, a mercaptan, a disulfide, a peroxide and a nitronate.

The remainder of the SAM molecule can be of any structure, so long as the SAM molecule is able to attach to a surface, and such that the remaining structure of the SAM molecule can promote an association between one or more other SAM molecules. For example, the SAM molecule may be comprised of any number of carbon atoms. In some embodiments, the SAM molecule is comprised of six to thirty-nine carbon atoms. In certain particular embodiments, the SAM molecules are comprised of eight, nine, ten, eleven, or twelve carbon atoms.

2. Exemplary Self-Assembled Monolayer Molecules

Exemplary self-assembled monolayer (SAM) molecules include, but are not limited to, molecules that include three parts—a middle portion and two end portions (one end portion covalently attend to each end of the middle portion). The middle portion, for example, may be composed of a carbon chain backbone structure of from six to thirty-nine or more carbon atoms. In this regard, the carbon chain backbone may be selected from the group consisting of hexane, heptane, octane, nonane, decane, undecane, dodecane, tridecane, tetradecane, pentadecane, hexadecane, heptadecane, octadecane, nonadecane, eicosane, uncosane, docosane, tricosane, tetracosane, petacosane, hexacosane, heptacosane, octacosane, nonacosane, triacontane, hentriacontane, dotriacontane, tritriacontane, tetratriacontane, pentatriacontane, hexatriacontane, heptatriacontane, octatriacontane, and nonatriacontane.

The SAM molecule may include either a branched or unbranched hydrocarbon chain, and may include any combination of single, double (alkene) and/or triple bonds (alkyne) in the carbon chain backbone. Further, the hydrocarbon chain may comprise a cyclic hydrocarbon group (e.g., pentanyl, hexanyl). The carbon chain backbone may be substituted or unsubstituted, wherein any one or more substituents may comprise one or more atoms (e.g., C, H, O, N, P, S, halogen) with such substituents being known to those of skill in the art. Any substitution that does not substantially alter the ability of the SAM molecule to form a SAM is contemplated. Non-limiting examples of such substituents include hydrogen, halogen, oxo (e.g., hydroxy, alkoxy, ester), cycloalkyl, carbonyl, acyl (including, for example, formyl, acetyl, propionyl, and the like), aryl, cyano, azido, amido, aminocarbonyl, amino, —NH-alkyl, —N(alkyl)₂, —NH-cycloalkyl, —N(cycloalkyl)₂, —NH-aryl, —N(aryl)₂, trialkylsilyloxy, acylamino, bis-acylamino, sulfo (e.g., thioether, thioester, sulfonamido, sulfonyl, silyloxy), NO, NO₂ and any combination of one or more of these groups.

As used herein, “alkyl” refers to a straight or branched chain comprising carbon-carbon single bonds, optionally including alkene or alkyne bonding, containing 1-30 carbons, preferably 1-6 carbons, and optionally substituted, as described above.

As used herein the term “cycloalkyl” refers to carbocycles or heterocycles of three or more ring atoms, the ring atoms of which may be optionally substituted with C, O, N or S and the ring atoms of which may comprise one or more substituents as described above.

The term “amino,” alone or in combination, is used interchangeably with “amine” and may refer to any one or more of the following: a primary (e.g., —NH₂), secondary (e.g., alkyl-NH—), tertiary (e.g., (alkyl)₂-N—), or quarternary (e.g., (alkyl)₃-N(+)—) amine radical.

The term “aryl” refers to a carbocyclic aromatic group or a heterocyclic aromatic group.

As used herein, a “halogen” refers to fluoro, chloro, bromo or iodo.

Exemplary end portion groups include those selected from the group consisting of amine, alcohol, carboxylic acid, phosphate, thiol, dithioic acid, carbamodithioic acid, phosphonic acid, carboxamide, N-hydroxycarboxamide, isocyanate, silane, methyldichlorosilane, trichlorosilane, chlorodimethylsilane, triethoxysilane, isocyanate, trimethoxysilane, bromide, chloride, and iodide. The end portions of a SAM molecule may be either identical or different. Included as possible end portions are those groups not otherwise set forth that do not substantially alter the ability of the SAM-forming molecule to form a SAM.

Particular examples of SAM molecules include the following, including any subsets of the following: 6-aminohexan-1-ol, 6-aminohexanoic acid, 6-aminohexyl dihydrogen phosphate, 6-aminohexan-1-thiol, 6-aminohexane(dithioc) acid, (6-aminohexyl)carbamodithioc acid, (6-aminohexyl)phosphonic acid, (6-amino)-N-hydroxyhexanamide, N-hydroxy-6-mercaptohexanamide, (6-mercaptohexyl)phosphonic acid, 6-mercaptohexan-1-ol, 6-mercaptohexanoic acid, 6-mercaptohexyl dihydrogen phosphate, 6-mercaptohexane(dithioc) acid, (6-mercaptohexyl)carbamodithioc acid, 6-hydroxyhexanoic acid, 6-hydroxyhexyl dihydrogen phosphate, 6-hydroxyhexane(dithioc) acid, (6-hydroxyhexyl)carbamodithioc acid, N,6-dihydroxyhexanamide, (6-hydroxyhexyl)phosphonic acid, 6-phosphonohexanoic acid, 6-(phosphonooxy)hexanoic acid, 6-mercapto-6-thioxohexanoic acid, 6-(hydroxyamino)-6-oxohexanoic acid, 6-[(mercaptocarbonothioyl)amino]hexanoic acid, [6-(hydroxyamino)-6-oxohexyl]phosphonic acid, [6-(phosphonooxy)hexyl]phosphonic acid, {6-[(mercaptocarbonothioyl)amino]hexyl}phosphonic acid, 6-phosphonohexane(dithioic) acid, [6-(phosphonooxy)hexyl]carbamodithioic acid, 6-(hydroxyamino)-6-oxohexyl dihydrogen phosphate, 6-(phosphonooxy)hexane(dithioic) acid, 6-(hydroxyamino)-6-oxohexane(dithioic) acid, [6-(hydroxyamino)-6-oxohexyl]carbarnodithioic acid, 6-[(mercaptocarbonothioyl)amino]hexane(dithioic) acid, 6-aminohexylmethyldichlorosilane, 6-mercaptohexylmethyldichlorosilane, 6-isocyanatohexylmethyldichlorosilane, 6-carboxyhexylmethyldichlorosilane, 6-hydroxyhexylmethyldichlorosilane, 6-iodohexylmethyldichlorosilane, 6-chlorohexylmethyldichlorosilane, 6-bromohexylmethyldichlorosilane, 6-aminohexyltrichlorosilane, 6-mercaptohexyltrichlorosilane, 6-isocyanatohexyltrichliorosilane, 6-carboxyhexyltrichlorosilane, 6-hydroxyhexyltrichlorosilane, 6-iodohexyltrichlorosilane, 6-chlorohexyltrichlorosilane, 6-bromohexyltrichlorosilane, 6-aminohexylchlorodimethylsilane, 6-mercaptohexylchlorodimethylsilane, 6-isocyanatohexylchlorodimethylsilane, 6-carboxyhexylchlorodimethylsilane, 6-hydroxyhexylchlorodimethylsilane, 6-iodohexylchlorodimethylsilane, 6-chlorohexylchlorodimethylsilane, 6-bromohexylchlorodimethylsilane, 6-aminohexyltriethoxysilane, 6-mercaptohexyltriethoxysilane, 6-isocyanatohexyltriethoxysilane, 6-carboxyhexyltriethoxysilane, 6-hydroxyhexyltriethoxysilane, 6-iodohexyltriethoxysilane, 6-chlorohexyltriethoxysilane, 6-bromohexyltriethoxysilane, 6-aminohexyltrimethoxysilane, 6-mercaptohexyltrimethoxysilane, 6-isocyanatohexyltrimethoxysilane, 6-carboxyhexyltrimethoxysilane, 6-hydroxyhexyltrimethoxysilane, 6-iodohexyltrimethoxysilane, 6-chlorohexyltrimethoxysilane, 6-bromohexyltrimethoxysilane, 6-bromohexan-1-ol, 6-bromohexanoic acid, 6-bromohexyl dihydrogen phosphate, 6-bromohexan-1-thiol, 6-bromohexane(dithioc) acid, (6-bromohexyl)carbamodithioc acid, (6-bromohexyl)phosphonic acid, (6-bromo)-N-hydroxyhexanamide, 6-chlorohexan-1-ol, 6-chlorohexanoic acid, 6-chlorohexyl dihydrogen phosphate, 6-chlorohexan-1-thiol, 6-chlorohexane(dithioc) acid, (6-chlorohexyl)carbamodithioc acid, (6-chlorohexyl)phosphonic acid, (6-chloro)-N-hydroxyhexanamide, 6-iodohexan-1-ol, 6-iodohexanoic acid, 6-iodohexyl dihydrogen phosphate, 6-iodohexan-1-thiol, 6-iodohexane(dithioc) acid, (6-iodohexyl)carbamodithioc acid, (6-iodohexyl)phosphonic acid, (6-iodo)-N-hydroxyhexanamide, 6-isocyanatohexan-1-ol, 6-isocyanatohexanoic acid, 6-isocyanatohexyl dihydrogen phosphate, 6-isocyanatohexan-1-thiol, 6-isocyanatohexane(dithioc) acid, (6-isocyanatohexyl)carbamodithioc acid, (6-isocyanatohexyl)phosphonic acid, (6-isocyanato)-N-hydroxyhexanamide, 7-aminoheptan-1-ol, 7-aminoheptanoic acid, 7-aminoheptyl dihydrogen phosphate, 7-aminoheptan-1-thiol, 7-aminoheptane(dithioc) acid, (7-aminoheptyl)carbamodithioc acid, (7-aminoheptyl)phosphonic acid, (7-amino)-N-hydroxyheptanamide, N-hydroxy-7-mercaptoheptanamide, (7-mercaptoheptyl)phosphonic acid, 7-mercaptoheptan-1-ol, 7-mercaptoheptanoic acid, 7-mercaptoheptyl dihydrogen phosphate, 7-mercaptoheptane(dithioc) acid, (7-mercaptoheptyl)carbamodithioc acid, 7-hydroxyheptanoic acid, 7-hydroxyheptyl dihydrogen phosphate, 7-hydroxyheptane(dithioc) acid, (7-hydroxyheptyl)carbamodithioc acid, N,7-dihydroxyheptanamide, (7-hydroxyheptyl)phosphonic acid, 7-phosphonoheptanoic acid, 7-(phosphonooxy)heptanoic acid, 7-mercapto-7-thioxoheptanoic acid, 7-(hydroxyamino)-7-oxoheptanoic acid, 7-[(mercaptocarbonothioyl)amino]heptanoic acid, [7-(hydroxyamino)-7-oxoheptyl]phosphonic acid, [7-(phosphonooxy)heptyl]phosphonic acid, {7-[(mercaptocarbonothioyl)amino]heptyl}phosphonic acid, 7-phosphonoheptane(dithioic) acid, [7-(phosphonooxy)heptyl]carbamodithioic acid, 7-(hydroxyamino)-7-oxoheptyl dihydrogen phosphate, 7-(phosphonooxy)heptane(dithioic) acid, 7-(hydroxyamino)-7-oxoheptane(dithioic) acid, [7-(hydroxyamino)-7-oxoheptyl]carbamodithioic acid, 7-[(mercaptocarbonothioyl)amino]heptane(dithioic) acid, 7-aminoheptylmethyldichlorosilane, 7-mercaptoheptylmethyldichlorosilane, 7-isocyanatoheptylmethyldichlorosilane, 7-carboxyheptylmethyldichlorosilane, 7-hydroxyheptylmethyldichlorosilane, 7-iodoheptylmethyldichlorosilane, 7-chloroheptylmethyldichlorosilane, 7-bromoheptylmethyldichlorosilane, 7-aminoheptyltrichlorosilane, 7-mercaptoheptyltrichlorosilane, 7-isocyanatoheptyltrichlorosilane, 7-carboxyheptyltrichlorosilane, 7-hydroxyheptyltrichlorosilane, 7-iodoheptyltrichlorosilane, 7-chloroheptyltrichlorosilane, 7-bromoheptyltrichlorosilane, 7-aminoheptylchlorodimethylsilane, 7-mercaptoheptylchlorodimethylsilane, 7-isocyanatoheptylchlorodimethylsilane, 7-carboxyheptylchlorodimethylsilane, 7-hydroxyheptylchlorodimethylsilane, 7-iodoheptylchlorodimethylsilane, 7-chloroheptylchlorodimethylsilane, 7-bromoheptylchlorodimethylsilane, 7-aminoheptyltriethoxysilane, 7-mercaptoheptyltriethoxysilane, 7-isocyanatoheptyltriethoxysilane, 7-carboxyheptyltriethoxysilane, 7-hydroxyheptyltriethoxysilane, 7-iodoheptyltriethoxysilane, 7-chloroheptyltriethoxysilane, 7-bromoheptyltriethoxysilane, 7-aminoheptyltrimethoxysilane, 7-mercaptoheptyltrimethoxysilane, 7-isocyanatoheptyltrimethoxysilane, 7-carboxyheptyltrimethoxysilane, 7-hydroxyheptyltrimethoxysilane, 7-iodoheptyltrimethoxysilane, 7-chloroheptyltrimethoxysilane, 7-bromoheptyltrimethoxysilane, 7-bromoheptan-1-ol, 7-bromoheptanoic acid, 7-bromoheptyl dihydrogen phosphate, 7-bromoheptan-1-thiol, 7-bromoheptane(dithioc) acid, (7-bromoheptyl)carbamodithioc acid, (7-bromoheptyl)phosphonic acid, (7-bromo)-N-hydroxyheptanamide, 7-chloroheptan-1-ol, 7-chloroheptanoic acid, 7-chloroheptyl dihydrogen phosphate, 7-chloroheptan-1-thiol, 7-chloroheptane(dithioc) acid, (7-chloroheptyl)carbamodithioc acid, (7-chloroheptyl)phosphonic acid, (7-chloro)-N-hydroxyheptanamide, 7-iodoheptan-1-ol, 7-iodoheptanoic acid, 7-iodoheptyl dihydrogen phosphate, 7-iodoheptan-1-thiol, 7-iodoheptane(dithioc) acid, (7-iodoheptyl)carbamodithioc acid, (7-iodoheptyl)phosphonic acid, (7-iodo)-N-hydroxyheptanamide, 7-isocyanatoheptan-1-ol, 7-isocyanatoheptanoic acid, 7-isocyanatoheptyl dihydrogen phosphate, 7-isocyanatoheptan-1-thiol, 7-isocyanatoheptane(dithioc) acid, (7-isocyanatoheptyl)carbamodithioc acid, (7-isocyanatoheptyl)phosphonic acid, (7-isocyanato)-N-hydroxyheptanamide, 8-aminooctan-1-ol, 8-aminooctanoic acid, 8-aminooctyl dihydrogen phosphate, 8-aminooctan-1-thiol, 8-aminooctane(dithioc) acid, (8-aminooctyl)carbamodithioc acid, (8-aminooctyl)phosphonic acid, (8-amino)-N-hydroxyoctanamide, N-hydroxy-8-mercaptooctanamide, (8-mercaptooctyl)phosphonic acid, 8-mercaptooctan-1-ol, 8-mercaptooctanoic acid, 8-mercaptooctyl dihydrogen phosphate, 8-mercaptooctane(dithioc) acid, (8-mercaptooctyl)carbamodithioc acid, 8-hydroxyoctanoic acid, 8-hydroxyoctyl dihydrogen phosphate, 8-hydroxyoctane(dithioc) acid, (8-hydroxyoctyl)carbamodithioc acid, N,8-dihydroxyoctanamide, (8-hydroxyoctyl)phosphonic acid, 8-phosphonooctanoic acid, 8-(phosphonooxy)octanoic acid, 8-mercapto-8-thioxooctanoic acid, 8-(hydroxyamino)-8-oxooctanoic acid, 8-[(mercaptocarbonothioyl)amino]octanoic acid, [8-(hydroxyamino)-8-oxooctyl]phosphonic acid, [8-(phosphonooxy)octyl]phosphonic acid, {8-[(mercaptocarbonothioyl)amino]octyl}phosphonic acid, 8-phosphonooctane(dithioic) acid, [8-(phosphonooxy)octyl]carbamodithioic acid, 8-(hydroxyamino)-8-oxooctyl dihydrogen phosphate, 8-(phosphonooxy)octane(dithioic) acid, 8-(hydroxyamino)-8-oxooctane(dithioic) acid, [8-(hydroxyamino)-8-oxooctyl]carbamodithioic acid, 8-[(mercaptocarbonothioyl)amino]octane(dithioic) acid, 8-aminooctylmethyldichlorosilane, 8-mercaptooctylmethyldichlorosilane, 8-isocyanatooctylmethyldichlorosilane, 8-carboxyoctylmethyldichlorosilane, 8-hydroxyoctylmethyldichlorosilane, 8-iodooctylmethyldichlorosilane, 8-chlorooctylmethyldichlorosilane, 8-bromooctylmethyldichlorosilane, 8-aminooctyltrichlorosilane, 8-mercaptooctyltrichlorosilane, 8-isocyanatooctyltrichlorosilane, 8-carboxyoctyltrichlorosilane, 8-hydroxyoctyltrichlorosilane, 8-iodooctyltrichlorosilane, 8-chlorooctyltrichlorosilane, 8-bromooctyltrichlorosilane, 8-aminooctylchlorodimethylsilane, 8-mercaptooctylchlorodimethylsilane, 8-isocyanatooctylchlorodimethylsilane, 8-carboxyoctylchlorodimethylsilane, 8-hydroxyoctylchlorodimethylsilane, 8-iodooctylchlorodimethylsilane, 8-chlorooctylchlorodimethylsilane, 8-bromooctylchlorodimethylsilane, 8-aminooctyltriethoxysilane, 8-mercaptooctyltriethoxysilane, 8-isocyanatooctyltriethoxysilane, 8-carboxyoctyltriethoxysilane, 8-hydroxyoctyltriethoxysilane, 8-iodooctyltriethoxysilane, 8-chlorooctyltriethoxysilane, 8-bromooctyltriethoxysilane, 8-aminooctyltrimethoxysilane, 8-mercaptooctyltrimethoxysilane, 8-isocyanatooctyltrimethoxysilane, 8-carboxyoctyltrimethoxysilane, 8-hydroxyoctyltrimethoxysilane, 8-iodooctyltrimethoxysilane, 8-chlorooctyltrimethoxysilane, 8-brornooctyltrimethoxysilane, 8-bromooctan-1-ol, 8-bromooctanoic acid, 8-bromooctyl dihydrogen phosphate, 8-bromooctan-1-thiol, 8-bromooctane(dithioc) acid, (8-bromooctyl)carbamodithioc acid, (8-bromooctyl)phosphonic acid, (8-bromo)-N-hydroxyoctanamide, 8-chlorooctan-1-ol, 8-chlorooctanoic acid, 8-chlorooctyl dihydrogen phosphate, 8-chlorooctan-1-thiol, 8-chlorooctane(dithioc) acid, (8-chlorooctyl)carbamodithioc acid, (8-chlorooctyl)phosphonic acid, (8-chloro)-N-hydroxyoctanamide, 8-iodooctan-1-ol, 8-iodooctanoic acid, 8-iodooctyl dihydrogen phosphate, 8-iodooctan-1-thiol, 8-iodooctane(dithioc) acid, (8-iodooctyl)carbamodithioc acid, (8-iodooctyl)phosphonic acid, (8-iodo)-N-hydroxyoctanamide, 8-isocyanatooctan-1-ol, 8-isocyanatooctanoic acid, 8-isocyanatooctyl dihydrogen phosphate, 8-isocyanatooctan-1-thiol, 8-isocyanatooctane(dithioc) acid, (8-isocyanatooctyl)carbamodithioc acid, (8-isocyanatooctyi)phosphonic acid, (8-isocyanato)-N-hydroxyoctanamide, 9-aminononan-1-ol, 9-aminononanoic acid, 9-aminononyl dihydrogen phosphate, 9-aminononan-1-thiol, 9-aminononane(dithioc) acid, (9-aminononyl)carbamodithioc acid, (9-aminononyl)phosphonic acid, (9-amino)-N-hydroxynonanamide, N-hydroxy-9-mercaptononanamide, (9-mercaptononyl)phosphonic acid, 9-mercaptononan-1-ol, 9-mercaptononanoic acid, 9-mercaptononyl dihydrogen phosphate, 9-mercaptononane(dithioc) acid, (9-mercaptononyl)carbamodithioc acid, 9-hydroxynonanoic acid, 9-hydroxynonyl dihydrogen phosphate, 9-hydroxynonane(dithioc) acid, (9-hydroxynonyl)carbamodithioc acid, N,9-dihydroxynonanamide, (9-hydroxynonyl)phosphonic acid, 9-phosphonononanoic acid, 9-(phosphonooxy)nonanoic acid, 9-mercapto-9-thioxononanoic acid, 9-(hydroxyamino)-9-oxononanoic acid, 9-[(mercaptocarbonothioyl)amino]nonanoic acid, [9-(hydroxyamino)-9-oxononyl]phosphonic acid, [9-(phosphonooxy)nonyl]phosphonic acid, {9-[(mercaptocarbonothioyl)amino]nonyl}phosphonic acid, 9-phosphonononane(dithioic) acid, [9-(phosphonooxy)nonyl]carbamodithioic acid, 9-(hydroxyamino)-9-oxononyl dihydrogen phosphate, 9-(phosphonooxy)nonane(dithioic) acid, 9-(hydroxyamino)-9-oxononane(dithioic) acid, [9-(hydroxyamino)-9-oxononyl]carbamodithioic acid, 9-[(mercaptocarbonothioyl)amino]nonane(dithioic) acid, 9-aminononylmethyldichlorosilane, 9-mercaptononylmethyldichlorosilane, 9-isocyanatononylmethyldichlorosilane, 9-carboxylionylmethyldichlorosilane, 9-hydroxynonylmethyldichlorosilane, 9-iodononylmethyldichlorosilane, 9-chlorononylmethyldichlorosilane, 9-bromononylmethyldichlorosilane, 9-aminononyltrichlorosilane, 9-mercaptononyltrichlorosilane, 9-isocyanatononyltrichlorosilane, 9-carboxynonyltrichlorosilane, 9-hydroxynonyltrichlorosilane, 9-iodononyltrichlorosilane, 9-chlorononyltrichlorosilane, 9-bromiononyltrichlorosilane, 9-aminononylchlorodimethylsilane, 9-mercaptononylchlorodimethylsilane, 9-isocyanatononylchlorodimethylsilane, 9-carboxynonylchlorodimethylsilane, 9-hydroxynonylchlorodimethylsilane, 9-iodononylchlorodimethylsilane, 9-chlorononylchlorodimethylsilane, 9-bromononylchlorodimethylsilane, 9-aminononyltriethoxysilane, 9-mercaptononyltriethoxysilane, 9-isocyanatononyltriethoxysilane, 9-carboxynonyltriethoxysilane, 9-hydroxynonyltriethoxysilane, 9-iodononyltriethoxysilane, 9-chlorononyltriethoxysilane, 9-bromononyltriethoxysilane, 9-aminononyltrimethoxysilane, 9-mercaptononyltrimethoxysilane, 9-isocyanatononyltrimethoxysilane, 9-carboxynonyltrimethoxysilane, 9-hydroxynonyltrimethoxysilane, 9-iodononyltrimethoxysilane, 9-chlorononyltrimethoxysilane, 9-bromononyltrimethoxysilane, 9-bromononan-1-ol, 9-bromononanoic acid, 9-bromononyl dihydrogen phosphate, 9-bromononan-1-thiol, 9-bromononane(dithioc) acid, (9-bromononyl)carbamodithioc acid, (9-bromononyl)phosphonic acid, (9-bromo)-N-hydroxynonanamide, 9-chlorononan-1-ol, 9-chlorononanoic acid, 9-chlorononyl dihydrogen phosphate, 9-chlorononan-1-thiol, 9-chlorononane(dithioc) acid, (9-chlorononyl)carbamodithioc acid, (9-chlorononyl)phosphonic acid, (9-chloro)-N-hydroxynonanamide, 9-iodononan-1-ol, 9-iodononanoic acid, 9-iodononyl dihydrogen phosphate, 9-iodononan-1-thiol, 9-iodononane(dithioc) acid, (9-iodononyl)carbamodithioc acid, (9-iodononyl)phosphonic acid, (9-iodo)-N-hydroxynonanamide, 9-isocyanatononan-1-ol, 9-isocyanatononanoic acid, 9-isocyanatononyl dihydrogen phosphate, 9-isocyanatononan-1-thiol, 9-isocyanatononane(dithioc) acid, (9-isocyanatononyl)carbamodithioc acid, (9-isocyanatononyl)phosphonic acid, (9-isocyanato)-N-hydroxynonanamide, 10-aminodecan-1-ol, 10-aminodecanoic acid, 10-aminodecyl dihydrogen phosphate, 10-aminodecan-1-thiol, 10-aminodecane(dithioc) acid, (10-aminodecyl)carbamodithioc acid, (10-aminodecyl)phosphonic acid, (10-amino)-N-hydroxydecanamide, N-hydroxy-10-mercaptodecanamide, (10-mercaptodecyl)phosphonic acid, 10-mercaptodecan-1-ol, 10-mercaptodecanoic acid, 10-mercaptodecyl dihydrogen phosphate, 10-mercaptodecane(dithioc) acid, (10-mercaptodecyl)carbamodithioc acid, 10-hydroxydecanoic acid, 10-hydroxydecyl dihydrogen phosphate, 10-hydroxydecane(dithioc) acid, (10-hydroxydecyl)carbamodithioc acid, N,10-dihydroxydecanamide, (10-hydroxydecyl)phosphonic acid, 10-phosphonodecanoic acid, 10-(phosphonooxy)decanoic acid, 10-mercapto-10-thioxodecanoic acid, 10-(hydroxyamino)-10-oxodecanoic acid, 10-[(mercaptocarbonothioyl)amino]decanoic acid, [10-(hydroxyamino)-10-oxodecyl]phosphonic acid, [10-(phosphonooxy)decyl]phosphonic acid, {10-[(mercaptocarbonothioyl)amino]decyl}phosphonic acid, 10-phosphonodecane(dithioic) acid, [10-(phosphonooxy)decyl]carbamodithioic acid, 10-(hydroxyamino)-10-oxodecyl dihydrogen phosphate, 10-(phosphonooxy)decane(dithioic) acid, 10-(hydroxyamino)-10-oxodecane(dithioic) acid, [10-(hydroxyamino)-10-oxodecyl]carbamodithioic acid, 10-[(mercaptocarbonothioyl)amino]decane(dithioic) acid, 10-aminodecylmethyldichlorosilane, 10-mercaptodecylmethyldichlorosilane, 10-isocyanatodecylmethyldichlorosilane, 10-carboxydecylmethyldichlorosilane, 10-hydroxydecylmethyldichlorosilane, 10-iododecylmethyldichlorosilane, 10-chlorodecylmethyldichlorosilane, 10-bromodecylmethyldichlorosilane, 10-aminodecyltrichlorosilane, 10-mercaptodecyltrichlorosilane, 10-isocyanatodecyltrichlorosilane, 10-carboxydecyltrichlorosilane, 10-hydroxydecyltrichlorosilane, 10-iododecyltrichlorosilane, 10-chlorodecyltrichlorosilane, 10-bromodecyltrichlorosilane, 10-aminodecylchlorodimethylsilane, 10-mercaptodecylchlorodimethylsilane, 10-isocyanatodecylchlorodimethylsilane, 10-carboxydecylchlorodimethylsilane, 10-hydroxydecylchlorodimethylsilane, 10-iododecylchlorodimethylsilane, 10-chlorodecylchlorodimethylsilane, 10-bromodecylchlorodimethylsilane, 10-aminodecyltriethoxysilane, 10-mercaptodecyltriethoxysilane, 10-isocyanatodecyltriethoxysilane, 10-carboxydecyltriethoxysilane, 10-hydroxydecyltriethoxysilane, 10-iododecyltriethoxysilane, 10-chlorodecyltriethoxysilane, 10-bromodecyltriethoxysilane, 10-aminodecyltrimethoxysilane, 10-mercaptodecyltrimethoxysilane, 10-isocyanatodecyltrimethoxysilane, 10-carboxydecyltrimethoxysilane, 10-hydroxydecyltrimethoxysilane, 10-iododecyltrimethoxysilane, 10-chlorodecyltrimethoxysilane, 10-bromodecyltrimethoxysilane, 10-bromodecan-1-ol, 10-bromodecanoic acid, 10-bromodecyl dihydrogen phosphate, 10-bromodecan-1-thiol, 10-bromodecane(dithioc) acid, (10-bromodecyl)carbamodithioc acid, (10-bromodecyl)phosphonic acid, (10-bromo)-N-hydroxydecanamide, 10-chlorodecan-1-ol, 10-chlorodecanoic acid, 10-chlorodecyl dihydrogen phosphate, 10-chlorodecan-1-thiol, 10-chlorodecane(dithioc) acid, (10-chlorodecyl)carbamodithioc acid, (10-chlorodecyl)phosphonic acid, (10-chloro)-N-hydroxydecanamide, 10-iododecan-1-ol, 10-iododecanoic acid, 10-iododecyl dihydrogen phosphate, 10-iododecan-1-thiol, 10-iododecane(dithioc) acid, (10-iododecyl)carbamodithioc acid, (10-iododecyl)phosphonic acid, (10-iodo)-N-hydroxydecanamide, 10-isocyanatodecan-1-ol, 10-isocyanatodecanoic acid, 10-isocyanatodecyl dihydrogen phosphate, 10-isocyanatodecan-1-thiol, 10-isocyanatodecane(dithioc) acid, (10-isocyanatodecyl)carbamodithioc acid, (10-isocyanatodecyl)phosphonic acid, (10-isocyanato)-N-hydroxydecanamide, 11-aminoundecan-1-ol, 11-aminoundecanoic acid, 1-aminoundecyl dihydrogen phosphate, 11-aminoundecan-1-thiol, 11-aminoundecane(dithioc) acid, (11-aminoundecyl)carbamodithioc acid, (11-aminoundecyl)phosphonic acid, (11-amino)-N-hydroxyundecanamide, N-hydroxy-11-mercaptoundecanamide, (11-mercaptoundecyl)phosphonic acid, 11-mercaptoundecan-1-ol, 11-mercaptoundecanoic acid, 11-mercaptoundecyl dihydrogen phosphate, 11-mercaptoundecane(dithioc) acid, (11-mercaptoundecyl)carbamodithioc acid, 11-hydroxyundecanoic acid, 11-hydroxyundecyl dihydrogen phosphate, 11-hydroxyundecane(dithioc) acid, (11-hydroxyundecyl)carbamodithioc acid, N,11-dihydroxyundecanamide, (11-hydroxyundecyl)phosphonic acid, 11-phosphonoundecanoic acid, 11-(phosphonooxy)undecanoic acid, 11-mercapto-11-thioxoundecanoic acid, 11-(hydroxyamino)-11-oxoundecanoic acid, 11-[(mercaptocarbonothioyl)amino]undecanoic acid, [11-(hydroxyamino)-11-oxoundecyl]phosphonic acid, [11-(phosphonooxy)undecyl]phosphonic acid, {11-[(mercaptocarbonothioyl)amino]undecyl}phosphonic acid, 11-phosphonoundecane(dithioic) acid, [11-(phosphonooxy)undecyl]carbamodithioic acid, 11-(hydroxyamino)-1-oxoundecyl dihydrogen phosphate, 11-(phosphonooxy)undecane(dithioic) acid, 11-(hydroxyamino)-11-oxoundecane(dithioic) acid, [11-(hydroxyamino)-11-oxoundecyl]carbamodithioic acid, 11-[(mercaptocarbonothioyl)amino]undecane(dithioic) acid, 11-aminoundecylmethyldichlorosilane, 11-mercaptoundecylmethyldichlorosilane, 11-isocyanatoundecylmethyldichlorosilane, 11-carboxyundecylmethyldichlorosilane, 11-hydroxyundecylmethyldichlorosilane, 11-iodoundecylmethyldichlorosilane, 11-chloroundecylmethyldichlorosilane, 11-bromoundecylmethyldichlorosilane, 11-aminoundecyltrichlorosilane, 11-mercaptoundecyltrichlorosilane, 11-isocyanatoundecyltrichlorosilane, 1-carboxyundecyltrichlorosilane, 11-hydroxyundecyltrichlorosilane, 11-iodoundecyltrichlorosilane, 11-chloroundecyltrichlorosilane, 11-bromoundecyltrichlorosilane, 11-aminoundecylchlorodimethylsilane, 11-mercaptoundecylchlorodimethylsilane, 11-isocyanatoundecylchlorodimethylsilane, 11-carboxyundecylchlorodimethylsilane, 11-hydroxyundecylchlorodimethylsilane, 11-iodoundecylchlorodimethylsilane, 11-chloroundecylchlorodimethylsilane, 11-bromoundecylchlorodimethylsilane, 11-aminoundecyltriethoxysilane, 11-mercaptoundecyltriethoxysilane, 11-isocyanatoundecyltriethoxysilane, 11-carboxyundecyltriethoxysilane, 11-hydroxyundecyltriethoxysilane, 11-iodoundecyltriethoxysilane, 11-chloroundecyltriethoxysilane, 11-bromoundecyltriethoxysilane, 11-aminoundecyltrimethoxysilane, 11-mercaptoundecyltrimethoxysilane, 11-isocyanatoundecyltrimethoxysilane, 11-carboxyundecyltrimethoxysilane, 11-hydroxyundecyltrimethoxysilane, 11-iodoundecyltrimethoxysilane, 11-chloroundecyltrimethoxysilane, 1-bromoundecyltrimethoxysilane, 1-bromoundecan-1-ol, 11-bromoundecanoic acid, 11-bromoundecyl dihydrogen phosphate, 11-bromoundecan-1-thiol, 11-bromoundecane(dithioc) acid, (11-bromoundecyl)carbamodithioc acid, (11-bromoundecyl)phosphonic acid, (11-bromo)-N-hydroxyundecanamide, 11-chloroundecan-1-ol, 11-chloroundecanoic acid, 11-chloroundecyl dihydrogen phosphate, 11-chloroundecan-1-thiol, 1′-chloroundecane(dithioc) acid, (1′-chloroundecyl)carbamodithioc acid, (11-chloroundecyl)phosphonic acid, (11-chloro)-N-hydroxyundecanamide, 11-iodoundecan-1-ol, 11-iodoundecanoic acid, 11-iodoundecyl dihydrogen phosphate, 11-iodoundecan-1-thiol, 11-iodoundecane(dithioc) acid, (11-iodoundecyl)carbamodithioc acid, (11-iodoundecyl)phosphonic acid, (11-iodo)-N-hydroxyundecanamide, 11-isocyanatoundecan-1-ol, 11-isocyanatoundecanoic acid, 11-isocyanatoundecyl dihydrogen phosphate, 11-isocyanatoundecan-1-thiol, 11-isocyanatoundecane(dithioc) acid, (11-isocyanatoundecyl)carbamodithioc acid, (11-isocyanatoundecyl)phosphonic acid, (11-isocyanato)-N-hydroxyundecanamide, 12-aminododecan-1-ol, 12-aminododecanoic acid, 12-aminododecyl dihydrogen phosphate, 12-aminododecan-1-thiol, 12-aminododecane(dithioc) acid, (12-aminododecyl)carbamodithioc acid, (12-aminododecyl)phosphonic acid, (12-amino)-N-hydroxydodecanamide, N-hydroxy-12-mercaptododecanamide, (12-mercaptododecyl)phosphonic acid, 12-mercaptododecan-1-ol, 12-mercaptododecanoic acid, 12-mercaptododecyl dihydrogen phosphate, 12-mercaptododecane(dithioc) acid, (12-mercaptododecyl)carbamodithioc acid, 12-hydroxydodecanoic acid, 12-hydroxydodecyl dihydrogen phosphate, 12-hydroxydodecane(dithioc) acid, (12-hydroxydodecyl)carbamodithioc acid, N,12-dihydroxydodecanamide, (12-hydroxydodecyl)phosphonic acid, 12-phosphonododecanoic acid, 12-(phosphonooxy)dodecanoic acid, 12-mercapto-12-thioxododecanoic acid, 12-(hydroxyamino)-12-oxododecanoic acid, 12-[(mercaptocarbonothioyl)amino]dodecanoic acid, [12-(hydroxyamino)-12-oxododecyl]phosphonic acid, [12-phosphonooxy)dodecyl]phosphonic acid, {12-[(mercaptocarbonothioyl)amino]dodecyl}phosphonic acid, 12-phosplionododecane(dithioic) acid, [12-(phosphonooxy)dodecyl]carbamodithioic acid, 12-(hydroxyamino)-12-oxododecyl dihydrogen phosphate, 12-(phosphonooxy)dodecane(dithioic) acid, 12-(hydroxyamino)-12-oxododecane(dithioic) acid, [12-(hydroxyamino)-12-oxododecyl]carbamodithioic acid, 12-[(mercaptocarbonothioyl)amino]dodecane(dithioic) acid, 12-aminododecylmethyldichlorosilane, 12-mercaptododecylmethyldichlorosilane, 12-isocyanatododecylmethyldichlorosilane, 12-carboxydodecylmethyldichlorosilane, 12-hydroxydodecylmethyldichlorosilane, 12-iodododecylmethyldichlorosilane, 12-chlorododecylmethyldichlorosilane, 12-bromododecylmethyldichlorosilane, 12-aminododecyltrichlorosilane, 12-mercaptododecyltrichlorosilane, 12-isocyanatododecyltrichlorosilane, 12-carboxydodecyltrichlorosilane, 12-hydroxydodecyltrichlorosilane, 12-iodododecyltrichlorosilane, 12-chlorododecyltrichlorosilane, 12-bromododecyltrichlorosilane, 12-aminododecylchlorodimethylsilane, 12-mercaptododecylchlorodimethylsilane, 12-isocyanatododecylchlorodimethylsilane, 12-carboxydodecylchlorodimethylsilane, 12-hydroxydodecylchlorodimethylsilane, 12-iodododecylchlorodimethylsilane, 12-chlorododecylchlorodimethylsilane, 12-bromododecylchlorodimethylsilane, 12-aminododecyltriethoxysilane, 12-mercaptododecyltriethoxysilane, 12-isocyanatododecyltriethoxysilane, 12-carboxydodecyltriethoxysilane, 12-hydroxydodecyltriethoxysilane, 12-iodododecyltriethoxysilane, 12-chlorododecyltriethoxysilane, 12-bromododecyltriethoxysilane, 12-aminododecyltrimethoxysilane, 12-mercaptododecyltrimethoxysilane, 12-isocyanatododecyltrimethoxysilane, 12-carboxydodecyltrimethoxysilane, 12-hydroxydodecyltrimethoxysilane, 12-iodododecyltrimethoxysilane, 12-chlorododecyltrimethoxysilane, 12-bromododecyltrimethoxysilane, 12-bromododecan-1-ol, 12-bromododecanoic acid, 12-bromododecyl dihydrogen phosphate, 12-bromododecan-1-thiol, 12-bromododecane(dithioc) acid, (12-bromododecyl)carbamodithioc acid, (12-bromododecyl)phosphonic acid, (12-bromo)-N-hydroxydodecanamide, 12-chlorododecan-1-ol, 12-chlorododecanoic acid, 12-chlorododecyl dihydrogen phosphate, 12-chlorododecan-1-thiol, 12-chlorododecane(dithioc) acid, (12-chlorododecyl)carbamodithioc acid, (12-chlorododecyl)phosphonic acid, (12-chloro)-N-hydroxydodecanamide, 12-iodododecan-1-ol, 12-iodododecanoic acid, 12-iodododecyl dihydrogen phosphate, 12-iodododecan-1-thiol, 12-iodododecane(dithioc) acid, (12-iodododecyl)carbamodithioc acid, (12-iodododecyl)phosphonic acid, (12-iodo)-N-hydroxydodecanamide, 12-isocyanatododecan-1-ol, 12-isocyanatododecanoic acid, 12-isocyanatododecyl dihydrogen phosphate, 12-isocyanatododecan-1-thiol, 12-isocyanatododecane(dithioc) acid, (12-isocyanatododecyl)carbamodithioc acid, (12-isocyanatododecyl)phosphonic acid, (12-isocyanato)-N-hydroxydodecanamide, 13-aminotridecan-1-ol, 13-aminotridecanoic acid, 13-aminotridecyl dihydrogen phosphate, 13-aminotridecan-1-thiol, 13-aminotridecane(dithioc) acid, (13-aminotridecyl)carbamodithioc acid, (13-aminotridecyl)phosphonic acid, (13-amino)-N-hydroxytridecanamide, N-hydroxy-13-mercaptotridecanamide, (13-mercaptotridecyl)phosphonic acid, 13-mercaptotridecan-1-ol, 13-mercaptotridecanoic acid, 13-mercaptotridecyl dihydrogen phosphate, 13-mercaptotridecane(dithioc) acid, (13-mercaptotridecyl)carbamodithioc acid, 13-hydroxytridecanoic acid, 13-hydroxytridecyl dihydrogen phosphate, 13-hydroxytridecane(dithioc) acid, (13-hydroxytridecyl)carbamodithioc acid, N,13-dihydroxytridecanamide, (13-hydroxytridecyl)phosphonic acid, 13-phosphonotridecanoic acid, 13-(phosphonooxy)tridecanoic acid, 13-mercapto-13-thioxotridecanoic acid, 13-(hydroxyamino)-13-oxotridecanoic acid, 13-[(mercaptocarbonothioyl)amino]tridecanoic acid, [13-(hydroxyamino)-13-oxotridecyl]phosphonic acid, [13-(phosphonooxy)tridecyl]phosphonic acid, {13-[(mercaptocarbonothioyl)amino]tridecyl}phosphonic acid, 13-phosphonotridecane(dithioic) acid, [13-(phosphonooxy)tridecyl]carbamodithioic acid, 13-(hydroxyamino)-13-oxotridecyl dihydrogen phosphate, 13-(phosphonooxy)tridecane(dithioic) acid, 13-(hydroxyamino)-13-oxotridecane(dithioic) acid, [13-(hydroxyamino)-13-oxotridecyl]carbamodithioic acid, 13-[(mercaptocarbonothioyl)amino]tridecane(dithioic) acid, 13-aminotridecylmethyldichlorosilane, 13-mercaptotridecylmethyldichlorosilane, 13-isocyanatotridecylmethyldichlorosilane, 13-carboxytridecylmethyldichlorosilane, 13-hydroxytridecylmethyldichlorosilane, 13-iodotridecylmethyldichlorosilane, 13-chlorotridecylmethyldichlorosilane, 13-bromotridecylmethyldichlorosilane, 13-aminotridecyltrichlorosilane, 13-mercaptotridec yltrichlorosilane, 13-isocyanatotridecyltrichlorosilane, 13-carboxytridecyltrichlorosilane, 13-hydroxytridecyltrichlorosilane, 13-iodotridecyltrichlorosilane, 13-chlorotridecyltrichlorosilane, 13-bromotridecyltrichlorosilane, 13-aminotridecylchlorodimethylsilane, 13-mercaptotridecylchlorodimethylsilane, 13-isocyanatotridecylchlorodimethylsilane, 13-carboxytridecylchlorodimethylsilane, 13-hydroxytridecylchlorodimethylsilane, 13-iodotridecylchlorodimethylsilane, 13-chlorotridecylchlorodimethylsilane, 13-bromotridecylchlorodimethylsilane, 13-aminotridecyltriethoxysilane, 13-mercaptotridecyltriethoxysilane, 13-isocyanatotridecyltriethoxysilane, 13-carboxytridecyltriethoxysilane, 13-hydroxytridecyltriethoxysilane, 13-iodotridecyltriethoxysilane, 13-chlorotridecyltriethoxysilane, 13-bromotridecyltriethoxysilane, 13-aminotridecyltrimethoxysilane, 13-mercaptotridecyltrimethoxysilane, 13-isocyanatotridecyltrimethoxysilane, 13-carboxytridecyltrimethoxysilane, 13-hydroxytridecyltrimethoxysilane, 13-iodotridecyltrimethoxysilane, 13-chlorotridecyltrimethoxysilane, 13-bromotridecyltrimethoxysilane, 13-bromotridecan-1-ol, 13-bromotridecanoic acid, 13-bromotridecyl dihydrogen phosphate, 13-bromotridecan-1-thiol, 13-bromotridecane(dithioc) acid, (13-bromotridecyl)carbamodithioc acid, (13-bromotridecyl)phosphonic acid, (13-bromo)-N-hydroxytridecanamide, 13-chlorotridecan-1-ol, 13-chlorotridecanoic acid, 13-chlorotridecyl dihydrogen phosphate, 13-chlorotridecan-1-thiol, 13-chlorotridecane(dithioc) acid, (13-chlorotridecyl)carbamodithioc acid, (13-chlorotridecyl)phosphonic acid, (13-chloro)-N-hydroxytridecanamide, 13-iodotridecan-1-ol, 13-iodotridecanoic acid, 13-iodotridecyl dihydrogen phosphate, 13-iodotridecan-1-thiol, 13-iodotridecane(dithioc) acid, (13-iodotridecyl)carbamodithioc acid, (13-iodotridecyl)phosphonic acid, (13-iodo)-N-hydroxytridecanamide, 13-isocyanatotridecan-1-ol, 13-isocyanatotridecanoic acid, 13-isocyanatotridecyl dihydrogen phosphate, 13-isocyanatotridecan-1-thiol, 13-isocyanatotridecane(dithioc) acid, (13-isocyanatotridecyl)carbamodithioc acid, (13-isocyanatotridecyl)phosphonic acid, (13-isocyanato)-N-hydroxytridecanamide, 14-aminotetradecan-1-ol, 14-aminotetradecanoic acid, 14-aminotetradecyl dihydrogen phosphate, 14-aminotetradecan-1-thiol, 14-aminotetradecane(dithioc) acid, (14-aminotetradecyl)carbamodithioc acid, (14-aminotetradecyl)phosphonic acid, (14-amino)-N-hydroxytetradecanamide, N-hydroxy-14-mercaptotetradecanamide, (14-mercaptotetradecyl)phosphonic acid, 14-mercaptotetradecan-1-ol, 14-mercaptotetradecanoic acid, 14-mercaptotetradecyl dihydrogen phosphate, 14-mercaptotetradecane(dithioc) acid, (14-mercaptotetradecyl)carbamodithioc acid, 14-hydroxytetradecanoic acid, 14-hydroxytetradecyl dihydrogen phosphate, 14-hydroxytetradecane(dithioc) acid, (14-hydroxytetradecyl)carbamodithioc acid, N,14-dihydroxytetradecanamide, (14-hydroxytetradecyl)phosphonic acid, 14-phosphonotetradecanoic acid, 14-(phosphonooxy)tetradecanoic acid, 14-mercapto-14-thioxotetradecanoic acid, 14-(hydroxyamino)-14-oxotetradecanoic acid, 14-[(mercaptocarbonothioyl)amino]tetradecanoic acid, [14-(hydroxyamino)-14-oxotetradecyl]phosphonic acid, [14-(phosphonooxy)tetradecyl]phosphonic acid, {14-[(mercaptocarbonothioyl)amino]tetradecyl}phosphonic acid, 14-phosphonotetradecane(dithioic) acid, [14-(phosphonooxy)tetradecyl]carbamodithioic acid, 14-(hydroxyamino)-14-oxotetradecyl dihydrogen phosphate, 14-(phosphonooxy)tetradecane(dithioic) acid, 14-(hydroxyamino)-14-oxotetradecane(dithioic) acid, [14-(hydroxyamino)-14-oxotetradecyl]carbamodithioic acid, 14-[(mercaptocarbonothioyl)amino]tetradecane(dithioic) acid, 14-aminotetradecylmethyldichlorosilane, 14-mercaptotetradecylmethyldichlorosilane, 14-isocyanatotetradecylmethyldichlorosilane, 14-carboxytetradecylmethyldichlorosilane, 14-hydroxytetradecylmethyldichlorosilane, 14-iodotetradecylmethyldichlorosilane, 14-chlorotetradecylmethyldichlorosilane, 14-bromotetradecylmethyldichlorosilane, 14-aminotetradecyltrichlorosilane, 14-mercaptotetradecyltrichlorosilane, 14-isocyanatotetradecyltrichlorosilane, 14-carboxytetradecyltrichlorosilane, 14-hydroxytetradecyltrichlorosilane, 14-iodotetradecyltrichlorosilane, 14-chlorotetradecyltrichlorosilane, 14-bromotetradecyltrichlorosilane, 14-aminotetradecylchlorodimethylsilane, 14-mercaptotetradecylchlorodimethylsilane, 14-isocyanatotetradecylchlorodimethylsilane, 14-carboxytetradecylchlorodimethylsilane, 14-hydroxytetradecylchlorodimethylsilane, 14-iodotetradecylchlorodimethylsilane, 14-chlorotetradecylchlorodimethylsilane, 14-bromotetradecylchlorodimethylsilane, 14-aminotetradecyltriethoxysilane, 14-mercaptotetradecyltriethoxysilane, 14-isocyanatotetradecyltriethoxysilane, 14-carboxytetradecyltriethoxysilane, 14-hydroxytetradecyltriethoxysilane, 14-iodotetradecyltriethoxysilane, 14-chlorotetradecyltriethoxysilane, 14-bromotetradecyltriethoxysilane, 14-aminotetradecyltrimethoxysilane, 14-mercaptotetradecyltrimethoxysilane, 14-isocyanatotetradecyltrimethoxysilane, 14-carboxytetradecyltrimethoxysilane, 14-hydroxytetradecyltrimethoxysilane, 14-iodotetradecyltrimethoxysilane, 14-chlorotetradecyltrimethoxysilane, 14-bromotetradecyltrimethoxysilane, 14-bromotetradecan-1-ol, 14-bromotetradecanoic acid, 14-bromotetradecyl dihydrogen phosphate, 14-bromotetradecan-1-thiol, 14-bromotetradecane(dithioc) acid, (14-bromotetradecyl)carbamodithioc acid, (14-bromotetradecyl)phosphonic acid, (14-bromo)-N-hydroxytetradecanamide, 14-chlorotetradecan-1-ol, 14-chlorotetradecanoic acid, 14-chlorotetradecyl dihydrogen phosphate, 14-chlorotetradecan-1-thiol, 14-chlorotetradecane(dithioc) acid, (14-chlorotetradecyl)carbamodithioc acid, (14-chlorotetradecyl)phosphonic acid, (14-chloro)-N-hydroxytetradecanamide, 14-iodotetradecan-1-ol, 14-iodotetradecanoic acid, 14-iodotetradecyl dihydrogen phosphate, 14-iodotetradecan-1-thiol, 14-iodotetradecane(dithioc) acid, (14-iodotetradecyl)carbamodithioc acid, (14-iodotetradecyl)phosphonic acid, (14-iodo)-N-hydroxytetradecanamide, 14-isocyanatotetradecan-1-ol, 14-isocyanatotetradecanoic acid, 14-isocyanatotetradecyl dihydrogen phosphate, 14-isocyanatotetradecan-1-thiol, 14-isocyanatotetradecane(dithioc) acid, (14-isocyanatotetradecyl)carbamodithioc acid, (14-isocyanatotetradecyl)phosphonic acid, (14-isocyanato)-N-hydroxytetradecanamide, 15-aminopentadecan-1-ol, 15-aminopentadecanoic acid, 15-aminopentadecyl dihydrogen phosphate, 15-aminopentadecan-1-thiol, 15-aminopentadecane(dithioc) acid, (15-aminopentadecyl)carbamodithioc acid, (15-aminopentadecyl)phosphonic acid, (15-amino)-N-hydroxypentadecanamide, N-hydroxy-15-mercaptopentadecanamide, (15-mercaptopentadecyl)phosphonic acid, 15-mercaptopentadecan-1-ol, 15-mercaptopentadecanoic acid, 15-mercaptopentadecyl dihydrogen phosphate, 15-mercaptopentadecane(dithioc) acid, (15-mercaptopentadecyl)carbamodithioc acid, 15-hydroxypentadecanoic acid, 15-hydroxypentadecyl dihydrogen phosphate, 15-hydroxypentadecane(dithioc) acid, (15-hydroxypentadecyl)carbamodithioc acid, N,15-dihydroxypentadecanamide, (15-hydroxypentadecyl)phosphonic acid, 15-phosphonopentadecanoic acid, 15-(phosphonooxy)pentadecanoic acid, 15-mercapto-15-thioxopentadecanoic acid, 15-(hydroxyamino)-15-oxopentadecanoic acid, 15-[(mercaptocarbonothioyl)amino]pentadecanoic acid, [15-(hydroxyamino)-15-oxopentadecyl]phosphonic acid, [15-(phosphonooxy)pentadecyl]phosphonic acid, {15-[(mercaptocarbonothioyl)amino]pentadecyl}phosphonic acid, 15-phosphonopentadecane(dithioic) acid, [15-(phosphonooxy)pentadecyl]carbamodithioic acid, 15-(hydroxyamino)-15-oxopentadecyl dihydrogen phosphate, 15-(phosphonooxy)pentadecane(dithioic) acid, 15-(hydroxyamino)-15-oxopentadecane(dithioic) acid, [15-(hydroxyamino)-15-oxopentadecyl]carbamodithioic acid, 15-[(mercaptocarbonothioyl)amino]pentadecane(dithioic) acid, 15-aminopentadecylmethyldichlorosilane, 15-mercaptopentadecylmethyldichlorosilane, 15-isocyanatopentadecylmethyldichlorosilane, 15-carboxypentadecylmethyldichlorosilane, 15-hydroxypentadecylmethyldichlorosilane, 15-iodopentadecylmethyldichlorosilane, 15-chloropentadecylmethyldichlorosilane, 15-bromopentadecylmethyldichlorosilane, 15-aminopentadecyltrichlorosilane, 15-mercaptopentadecyltrichlorosilane, 15-isocyanatopentadecyltrichlorosilane, 15-carboxypentadecyltrichlorosilane, 15-hydroxypentadecyltrichlorosilane, 15-iodopentadecyltrichlorosilane, 15-chloropentadecyltrichlorosilane, 15-bromopentadecyltrichlorosilane, 15-aminopentadecylchlorodimethylsilane, 15-mercaptopentadecylchlorodimethylsilane, 15-isocyanatopentadecylchlorodimethylsilane, 15-carboxypentadecylchlorodimethylsilane, 15-hydroxypentadecylchlorodimethylsilane, 15-iodopentadecylchlorodimethylsilane, 15-chloropentadecylchlorodimethylsilane, 15-bromopentadecylchlorodimethylsilane, 15-aminopentadecyltriethoxysilane, 15-mercaptopentadecyltriethoxysilane, 15-isocyanatopentadecyltriethoxysilane, 15-carboxypentadecyltriethoxysilane, 15-hydroxypentadecyltriethoxysilane, 15-iodopentadecyltriethoxysilane, 15-chloropentadecyltriethoxysilane, 15-bromopentadecyltriethoxysilane, 15-aminopentadecyltrimethoxysilane, 15-mercaptopentadecyltrimethoxysilane, 15-isocyanatopentadecyltrimethoxysilane, 15-carboxypentadecyltrimethoxysilane, 15-hydroxypentadecyltrimethoxysilane, 15-iodopentadecyltrimethoxysilane, 15-chloropentadecyltrimethoxysilane, 15-bromopentadecyltrimethoxysilane, 15-bromopentadecan-1-ol, 15-bromopentadecanoic acid, 15-bromopentadecyl dihydrogen phosphate, 15-bromopentadecan-1-thiol, 15-bromopentadecane(dithioc) acid, (15-bromopentadecyl)carbamodithioc acid, (15-bromopentadecyl)phosphonic acid, (15-bromo)-N-hydroxypentadecanamide, 15-chloropentadecan-1-ol, 15-chloropentadecanoic acid, 15-chloropentadecyl dihydrogen phosphate, 15-chloropentadecan-1-thiol, 15-chloropentadecane(dithioc) acid, (15-chloropentadecyl)carbamodithioc acid, (15-chloropentadecyl)phosphonic acid, (15-chloro)-N-hydroxypentadecanamide, 15-iodopentadecan-1-ol, 15-iodopentadecanoic acid, 15-iodopentadecyl dihydrogen phosphate, 15-iodopentadecan-1-thiol, 15-iodopentadecane(dithioc) acid, (15-iodopentadecyl)carbamodithioc acid, (15-iodopentadecyl)phosphonic acid, (15-iodo)-N-hydroxypentadecanamide, 15-isocyanatopentadecan-1-ol, 15-isocyanatopentadecanoic acid, 15-isocyanatopentadecyl dihydrogen phosphate, 15-isocyanatopentadecan-1-thiol, 15-isocyanatopentadecane(dithioc) acid, (15-isocyanatopentadecyl)carbamodithioc acid, (15-isocyanatopentadecyl)phosphonic acid, (15-isocyanato)-N-hydroxypentadecanamide, 16-aminohexadecan-1-ol, 16-aminohexadecanoic acid, 16-aminohexadecyl dihydrogen phosphate, 16-aminohexadecan-1-thiol, 16-aminohexadecane(dithioc) acid, (16-aminohexadecyl)carbamodithioc acid, (16-aminohexadecyl)phosphonic acid, (16-amino)-N-hydroxyhexadecanamide, N-hydroxy-16-mercaptohexadecanamide, (16-mercaptohexadecyl)phosphonic acid, 16-mercaptohexadecan-1-ol, 16-mercaptohexadecanoic acid, 16-mercaptohexadecyl dihydrogen phosphate, 16-mercaptohexadecane(dithioc) acid, (16-mercaptohexadecyl)carbamodithioc acid, 16-hydroxyhexadecanoic acid, 16-hydroxyhexadecyl dihydrogen phosphate, 16-hydroxyhexadecane(dithioc) acid, (16-hydroxyhexadecyl)carbamodithioc acid, N,16-dihydroxyhexadecanamide, (16-hydroxyhexadecyl)phosphonic acid, 16-phosphonohexadecanoic acid, 16-(phosphonooxy)hexadecanoic acid, 16-mercapto-16-thioxohexadecanoic acid, 16-(hydroxyamino)-16-oxohexadecanoic acid, 16-[(mercaptocarbonothioyl)amino]hexadecanoic acid, [16-(hydroxyamino)-16-oxohexadecyl]phosphonic acid, [16-(phosphonooxy)hexadecyl]phosphonic acid, {16-[(mercaptocarbonothioyl)amino]hexadecyl}phosphonic acid, 16-phosphonohexadecane(dithioic) acid, [16-(phosphonooxy)hexadecyl]carbamodithioic acid, 16-(hydroxyamino)-16-oxohexadecyl dihydrogen phosphate, 16-(phosphonooxy)hexadecane(dithioic) acid, 16-(hydroxyamino)-16-oxohexadecane(dithioic) acid, [16-(hydroxyamino)-16-oxohexadecyl]carbamodithioic acid, 16-[(mercaptocarbonothioyl)amino]hexadecane(dithioic) acid, 16-aminohexadecylmethyldichlorosilane, 16-mercaptohexadecylmethyldichlorosilane, 16-isocyanatohexadecylmethyldichlorosilane, 16-carboxyhexadecylmethyldichlorosilane, 16-hydroxyhexadecylmethyldichlorosilane, 16-iodohexadecylmethyldichlorosilane, 16-chlorohexadecylmethyldichlorosilane, 16-bromohexadecylmethyldichlorosilane, 16-aminohexadecyltrichlorosilane, 16-mercaptohexadecyltrichlorosilane, 16-isocyanatohexadecyltrichlorosilane, 16-carboxyhexadecyltrichlorosilane, 16-hydroxyhexadecyltrichlorosilane, 16-iodohexadecyltrichlorosilane, 16-chlorohexadecyltrichlorosilane, 16-bromohexadecyltrichlorosilane, 16-aminohexadecylchlorodimethylsilane, 16-mercaptohexadecylchlorodimethylsilane, 16-isocyanatohexadecylchlorodimethylsilane, 16-carboxyhexadecylchlorodimethylsilane, 16-hydroxyhexadecylchlorodimethylsilane, 16-iodohexadecylchlorodimethylsilane, 16-chlorohexadecylchlorodimethylsilane, 16-bromohexadecylchlorodimethylsilane, 16-aminohexadecyltriethoxysilane, 16-mercaptohexadecyltriethoxysilane, 16-isocyanatohexadecyltriethoxysilane, 16-carboxyhexadecyltriethoxysilane, 16-hydroxyhexadecyltriethoxysilane, 16-iodohexadecyltriethoxysilane, 16-chlorohexadecyltriethoxysilane, 16-bromohexadecyltriethoxysilane, 16-aminohexadecyltrimethoxysilane, 16-mercaptohexadecyltrimethoxysilane, 16-isocyanatohexadecyltrimethoxysilane, 16-carboxyhexadecyltrimethoxysilane, 16-hydroxyhexadecyltrimethoxysilane, 16-iodohexadecyltrimethoxysilane, 16-chlorohexadecyltrimethoxysilane, 16-bromohexadecyltrimethoxysilane, 16-bromohexadecan-1-ol, 16-bromohexadecanoic acid, 16-bromohexadecyl dihydrogen phosphate, 16-bromohexadecan-1-thiol, 16-bromohexadecane(dithioc) acid, (16-bromohexadecyl)carbamodithioc acid, (16-bromohexadecyl)phosphonic acid, (16-bromo)-N-hydroxyhexadecanamide, 16-chlorohexadecan-1-ol, 16-chlorohexadecanoic acid, 16-chlorohexadecyl dihydrogen phosphate, 16-chlorohexadecan-1-thiol, 16-chlorohexadecane(dithioc) acid, (16-chlorohexadecyl)carbamodithioc acid, (16-chlorohexadecyl)phosphonic acid, (16-chloro)-N-hydroxyhexadecanamide, 16-iodohexadecan-1-ol, 16-iodohexadecanoic acid, 16-iodohexadecyl dihydrogen phosphate, 16-iodohexadecan-1-thiol, 16-iodohexadecane(dithioc) acid, (16-iodohexadecyl)carbamodithioc acid, (16-iodohexadecyl)phosphonic acid, (16-iodo)-N-hydroxyhexadecanamide, 16-isocyanatohexadecan-1-ol, 16-isocyanatohexadecanoic acid, 16-isocyanatohexadecyl dihydrogen phosphate, 16-isocyanatohexadecan-1-thiol, 16-isocyanatohexadecane(dithioc) acid, (16-isocyanatohexadecyl)carbamodithioc acid, (16-isocyanatohexadecyl)phosphonic acid, (16-isocyanato)-N-hydroxyhexadecanamide, 17-aminoheptadecan-1-ol, 17-aminoheptadecanoic acid, 17-aminoheptadecyl dihydrogen phosphate, 17-aminoheptadecan-1-thiol, 17-aminoheptadecane(dithioc) acid, (17-aminoheptadecyl)carbamodithioc acid, (17-aminoheptadecyl)phosphonic acid, (17-amino)-N-hydroxyheptadecanamide, N-hydroxy-17-mercaptoheptadecanamide, (17-mercaptoheptadecyl)phosphonic acid, 17-mercaptoheptadecan-1-ol, 17-mercaptoheptadecanoic acid, 17-mercaptoheptadecyl dihydrogen phosphate, 17-mercaptoheptadecane(dithioc) acid, (17-mercaptoheptadecyl)carbamodithioc acid, 17-hydroxyheptadecanoic acid, 17-hydroxyheptadecyl dihydrogen phosphate, 17-hydroxyheptadecane(dithioc) acid, (17-hydroxyheptadecyl)carbamodithioc acid, N,17-dihydroxyheptadecanamide, (17-hydroxyheptadecyl)phosphonic acid, 17-phosphonoheptadecanoic acid, 17-(phosphonooxy)heptadecanoic acid, 17-mercapto-17-thioxoheptadecanoic acid, 17-(hydroxyamino)-17-oxoheptadecanoic acid, 17-[(mercaptocarbonothioyl)amino]heptadecanoic acid, [17-(hydroxyamino)-17-oxoheptadecyl]phosphonic acid, [17-(phosphonooxy)heptadecyl]phosphonic acid, {17-[(mercaptocarbonothioyl)amino]heptadecyl}phosphonic acid, 17-phosphonoheptadecane(dithioic) acid, [17-(phosphonooxy)heptadecyl]carbamodithioic acid, 17-(hydroxyamino)-17-oxoheptadecyl dihydrogen phosphate, 17-(phosphonooxy)heptadecane(dithioic) acid, 17-(hydroxyamino)-17-oxoheptadecane(dithioic) acid, [17-(hydroxyamino)-17-oxoheptadecyl]carbamodithioic acid, 17-[(mercaptocarbonothioyl)amino]heptadecane(dithioic) acid, 17-aminoheptadecylmethyldichlorosilane, 17-mercaptoheptadecylmethyldichlorosilane, 17-isocyanatoheptadecylmethyldichlorosilane, 17-carboxyheptadecylmethyldichlorosilane, 17-hydroxyheptadecylmethyldichlorosilane, 17-iodoheptadecylmethyldichlorosilane, 17-chloroheptadecylmethyldichlorosilane, 17-bromoheptadecylmethyldichlorosilane, 17-aminoheptadecyltrichlorosilane, 17-mercaptoheptadecyltrichlorosilane, 17-isocyanatoheptadecyltrichlorosilane, 17-carboxyheptadecyltrichlorosilane, 17-hydroxyheptadecyltrichlorosilane, 17-iodoheptadecyltrichlorosilane, 17-chloroheptadecyltrichlorosilane, 17-bromoheptadecyltrichlorosilane, 17-aminoheptadecylchlorodimethylsilane, 17-mercaptoheptadecylchlorodimethylsilane, 17-isocyanatoheptadecylchlorodimethylsilane, 17-carboxyheptadecylchlorodimethylsilane, 17-hydroxyheptadecylchlorodimethylsilane, 17-iodoheptadecylchlorodimethylsilane, 17-chloroheptadecylchlorodimethylsilane, 17-bromoheptadecylchlorodimethylsilane, 17-aminoheptadecyltriethoxysilane, 17-mercaptoheptadecyltriethoxysilane, 17-isocyanatoheptadecyltriethoxysilane, 17-carboxyheptadecyltriethoxysilane, 17-hydroxyheptadecyltriethoxysilane, 17-iodoheptadecyltriethoxysilane, 17-chloroheptadecyltriethoxysilane, 17-bromoheptadecyltriethoxysilane, 17-aminoheptadecyltrimethoxysilane, 17-mercaptoheptadecyltrimethoxysilane, 17-isocyanatoheptadecyltrimethoxysilane, 17-carboxyheptadecyltrimethoxysilane, 17-hydroxyheptadecyltrimethoxysilane, 17-iodoheptadecyltrimethoxysilane, 17-chloroheptadecyltrimethoxysilane, 17-bromoheptadecyltrimethoxysilane, 17-bromoheptadecan-1-ol, 17-bromoheptadecanoic acid, 17-bromoheptadecyl dihydrogen phosphate, 17-bromoheptadecan-1-thiol, 17-bromoheptadecane(dithioc) acid, (17-bromoheptadecyl)carbamodithioc acid, (17-bromoheptadecyl)phosphonic acid, (17-bromo)-N-hydroxyheptadecanamide, 17-chloroheptadecan-1-ol, 17-chloroheptadecanoic acid, 17-chloroheptadecyl dihydrogen phosphate, 17-chloroheptadecan-1-thiol, 17-chloroheptadecane(dithioc) acid, (17-chloroheptadecyl)carbamodithioc acid, (17-chloroheptadecyl)phosphonic acid, (17-chloro)-N-hydroxyheptadecanamide, 17-iodoheptadecan-1-ol, 17-iodoheptadecanoic acid, 17-iodoheptadecyl dihydrogen phosphate, 17-iodoheptadecan-1-thiol, 17-iodoheptadecane(dithioc) acid, (17-iodoheptadecyl)carbamodithioc acid, (17-iodoheptadecyl)phosphonic acid, (17-iodo)-N-hydroxyheptadecanamide, 17-isocyanatoheptadecan-1-ol, 17-isocyanatoheptadecanoic acid, 17-isocyanatoheptadecyl dihydrogen phosphate, 17-isocyanatoheptadecan-1-thiol, 17-isocyanatoheptadecane(dithioc) acid, (17-isocyanatoheptadecyl)carbamodithioc acid, (17-isocyanatoheptadecyl)phosphonic acid, (17-isocyanato)-N-hydroxyheptadecanamide, 18-aminooctadecan-1-ol, 18-aminooctadecanoic acid, 18-aminooctadecyl dihydrogen phosphate, 18-aminooctadecan-1-thiol, 18-aminooctadecane(dithioc) acid, (18-aminooctadecyl)carbamodithioc acid, (18-aminooctadecyl)phosphonic acid, (18-amino)-N-hydroxyoctadecanamide, N-hydroxy-18-mercaptooctadecanamide, (18-mercaptooctadecyl)phosphonic acid, 18-mercaptooctadecan-1-ol, 18-mercaptooctadecanoic acid, 18-mercaptooctadecyl dihydrogen phosphate, 18-mercaptooctadecane(dithioc) acid, (18-mercaptooctadecyl)carbamodithioc acid, 18-hydroxyoctadecanoic acid, 18-hydroxyoctadecyl dihydrogen phosphate, 18-hydroxyoctadecane(dithioc) acid, (18-hydroxyoctadecyl)carbamodithioc acid, N,18-dihydroxyoctadecanamide, (18-hydroxyoctadecyl)phosphonic acid, 18-phosphonooctadecanoic acid, 18-(phosphonooxy)octadecanoic acid, 18-mercapto-18-thioxooctadecanoic acid, 18-(hydroxyamino)-18-oxooctadecanoic acid, 18-[(mercaptocarbonothioyl)amino]octadecanoic acid, [18-(hydroxyamino)-18-oxooctadecyl]phosphonic acid, [18-(phosphonooxy)octadecyl]phosphonic acid, {18-[(mercaptocarbonothioyl)amino]octadecyl}phosphonic acid, 18-phosphonooctadecane(dithioic) acid, [18-(phosphonooxy)octadecyl]carbamodithioic acid, 18-(hydroxyamino)-18-oxooctadecyl dihydrogen phosphate, 18-(phosphonooxy)octadecane(dithioic) acid, 18-(hydroxyamino)-18-oxooctadecane(dithioic) acid, [18-(hydroxyamino)-18-oxooctadecyl]carbamodithioic acid, 18-[(mercaptocarbonothioyl)amino]octadecane(dithioic) acid, 18-aminooctadecylmethyldichlorosilane, 18-mercaptooctadecylmethyldichlorosilane, 18-isocyanatooctadecylmethyldichlorosilane, 18-carboxyoctadecylmethyldichlorosilane, 18-hydroxyoctadecylmethyldichlorosilane, 18-iodooctadecylmethyldichlorosilane, 18-chlorooctadecylmethyldichlorosilane, 18-bromooctadecylmethyldichlorosilane, 18-aminooctadecyltrichlorosilane, 18-mercaptooctadecyltrichlorosilane, 18-isocyanatooctadecyltrichlorosilane, 18-carboxyoctadecyltrichlorosilane, 18-hydroxyoctadecyltrichlorosilane, 18-iodooctadecyltrichlorosilane, 18-chlorooctadecyltrichlorosilane, 18-bromooctadecyltrichlorosilane, 18-aminooctadecylchlorodimethylsilane, 18-mercaptooctadecylchlorodimethylsilane, 18-isocyanatooctadecylchlorodimethylsilane, 18-carboxyoctadecylchlorodimethylsilane, 18-hydroxyoctadecylchlorodimethylsilane, 18-iodooctadecylchlorodimethylsilane, 18-chlorooctadecylchlorodimethylsilane, 18-bromooctadecylchlorodimethylsilane, 18-aminooctadecyltriethoxysilane, 18-mercaptooctadecyltriethoxysilane, 18-isocyanatooctadecyltriethoxysilane, 18-carboxyoctadecyltriethoxysilane, 18-hydroxyoctadecyltriethoxysilane, 18-iodooctadecyltriethoxysilane, 18-chlorooctadecyltriethoxysilane, 18-bromooctadecyltriethoxysilane, 18-aminooctadecyltrimethoxysilane, 18-mercaptooctadecyltrimethoxysilane, 18-isocyanatooctadecyltrimethoxysilane, 18-carboxyoctadecyltrimethoxysilane, 18-hydroxyoctadecyltrimethoxysilane, 18-iodooctadecyltrimethoxysilane, 18-chlorooctadecyltrimethoxysilane, 18-bromooctadecyltrimethoxysilane, 18-bromooctadecan-1-ol, 18-bromooctadecanoic acid, 18-bromooctadecyl dihydrogen phosphate, 18-bromooctadecan-1-thiol, 18-bromooctadecane(dithioc) acid, (18-bromooctadecyl)carbamodithioc acid, (18-bromooctadecyl)phosphonic acid, (18-bromo)-N-hydroxyoctadecanamide, 18-chlorooctadecan-1-ol, 18-chlorooctadecanoic acid, 18-chlorooctadecyl dihydrogen phosphate, 18-chlorooctadecan-1-thiol, 18-chlorooctadecane(dithioc) acid, (18-chlorooctadecyl)carbamodithioc acid, (18-chlorooctadecyl)phosphonic acid, (18-chloro)-N-hydroxyoctadecanamide, 18-iodooctadecan-1-ol, 18-iodooctadecanoic acid, 18-iodooctadecyl dihydrogen phosphate, 18-iodooctadecan-1-thiol, 18-iodooctadecane(dithioc) acid, (18-iodooctadecyl)carbamodithioc acid, (18-iodooctadecyl)phosphonic acid, (18-iodo)-N-hydroxyoctadecanamide, 18-isocyanatooctadecan-1-ol, 18-isocyanatooctadecanoic acid, 18-isocyanatooctadecyl dihydrogenphosphate, 18-isocyanatooctadecan-1-thiol, 18-isocyanatooctadecane(dithioc) acid, (18-isocyanatooctadecyl)carbamodithioc acid, (18-isocyanatooctadecyl)phosphonic acid, (18-isocyanato)-N-hydroxyoctadecanamide, 19-aminononadecan-1-ol, 19-aminononadecanoic acid, 19-aminononadecyl dihydrogen phosphate, 19-aminononadecan-1-thiol, 19-aminononadecane(dithioc) acid, (19-aminononadecyl)carbamodithioc acid, (19-aminononadecyl)phosphonic acid, (19-amino)-N-hydroxynonadecanamide, N-hydroxy-19-mercaptononadecanamide, (19-mercaptononadecyl)phosphonic acid, 19-mercaptononadecan-1-ol, 19-mercaptononadecanoic acid, 19-mercaptononadecyl dihydrogen phosphate, 19-mercaptononadecane(dithioc) acid, (19-mercaptononadecyl)carbamodithioc acid, 19-hydroxynonadecanoic acid, 19-hydroxynonadecyl dihydrogen phosphate, 19-hydroxynonadecane(dithioc) acid, (19-hydroxynonadecyl)carbamodithioc acid, N,19-dihydroxynonadecanamide, (19-hydroxynonadecyl)phosphonic acid, 19-phosphonononadecanoic acid, 19-(phosphonooxy)nonadecanoic acid, 19-mercapto-19-thioxononadecanoic acid, 19-(hydroxyamino)-19-oxononadecanoic acid, 19[(mercaptocarbonothioyl)amino]nonadecanoic acid, [19-(hydroxyamino)-19-oxononadecyl]phosphonic acid, [19-(phosphonooxy)nonadecyl]phosphonic acid, {19-[(mercaptocarbonothioyl)amino]nonadecyl}phosphonic acid, 19-phosphonononadecane(dithioic) acid, [19-(phosphonooxy)nonadecyl]carbamodithioic acid, 19-(hydroxyamino)-19-oxononadecyl dihydrogen phosphate, 19-(phosphonooxy)nonadecane(dithioic) acid, 19-(hydroxyamino)-19-oxononadecane(dithioic) acid, [19-(hydroxyamino)-19-oxononadecyl]carbamodithioic acid, 19-[(mercaptocarbonothioyl)amino]nonadecane(dithioic) acid, 19-aminononadecylmethyldichlorosilane, 19-mercaptononadecylmethyldichlorosilane, 19-isocyanatononadecylmethyldichlorosilane, 19-carboxynonadecylmethyldichlorosilane, 19-hydroxynonadecylmethyldichlorosilane, 19-iodononadecylmethyldichlorosilane, 19-chlorononadecylmethyldichlorosilane, 19-bromononadecylmethyldichlorosilane, 19-aminononadecyltrichlorosilane, 19-mercaptononadecyltrichlorosilane, 19-isocyanatononadecyltrichlorosilane, 19-carboxynonadecyltrichlorosilane, 19-hydroxynonadecyltrichlorosilane, 19-iodononadecyltrichlorosilane, 19-chlorononadecyltrichlorosilane, 19-bromononadecyltrichlorosilane, 19-aminononadecylchlorodimethylsilane, 19-mercaptononadecylchlorodimethylsilane, 19-isocyanatononadecylchlorodimethylsilane, 19-carboxynonadecylchlorodimethylsilane, 19-hydroxynonadecylchlorodimethylsilane, 19-iodononadecylchlorodimethylsilane, 19-chlorononadecylchlorodimethylsilane, 19-bromononadecylchlorodimethylsilane, 19-aminononadecyltriethoxysilane, 19-mercaptononadecyltriethoxysilane, 19-isocyanatononadecyltriethoxysilane, 19-carboxynonadecyltriethoxysilane, 19-hydroxynonadecyltriethoxysilane, 19-iodononadecyltriethoxysilane, 19-chlorononadecyltriethoxysilane, 19-bromononadecyltriethoxysilane, 19-aminononadecyltrimethoxysilane, 19-mercaptononadecyltrimethoxysilane, 19-isocyanatononadecyltrimethoxysilane, 19-carboxynonadecyltrimethoxysilane, 19-hydroxynonadecyltrimethoxysilane, 19-iodononadecyltrimethoxysilane, 19-chlorononadecyltrimethoxysilane, 19-bromononadecyltrimethoxysilane, 19-bromononadecan-1-ol, 19-bromononadecanoic acid, 19-bromononadecyl dihydrogen phosphate, 19-bromononadecan-1-thiol, 19-bromononadecane(dithioc) acid, (19-bromononadecyl)carbamodithioc acid, (19-bromononadecyl)phosphonic acid, (19-bromo)-N-hydroxynonadecanamide, 19-chlorononadecan-1-ol, 19-chlorononadecanoic acid, 19-chlorononadecyl dihydrogen phosphate, 19-chlorononadecan-1-thiol, 19-chlorononadecane(dithioc) acid, (19-chlorononadecyl)carbamodithioc acid, (19-chlorononadecyl)phosphonic acid, (19-chloro)-N-hydroxynonadecanamide, 19-iodononadecan-1-ol, 19-iodononadecanoic acid, 19-iodononadecyl dihydrogen phosphate, 19-iodononadecan-1-thiol, 19-iodononadecane(dithioc) acid, (19-iodononadecyl)carbamodithioc acid, (19-iodononadecyl)phosphonic acid, (19-iodo)-N-hydroxynonadecanamide, 19-isocyanatononadecan-1-ol, 19-isocyanatononadecanoic acid, 19-isocyanatononadecyl dihydrogen phosphate, 19-isocyanatononadecan-1-thiol, 19-isocyanatononadecane(dithioc) acid, (19-isocyanatononadecyl)carbamodithioc acid, (19-isocyanatononadecyl)phosphonic acid, (19-isocyanato)-N-hydroxynonadecanamide, 20-aminoeicosan-1-ol, 20-aminoeicosanoic acid, 20-aminoeicosyl dihydrogen phosphate, 20-aminoeicosan-1-thiol, 20-aminoeicosane(dithioc) acid, (20-aminoeicosyl)carbamodithioc acid, (20-aminoeicosyl)phosphonic acid, (20-amino)-N-hydroxyeicosanamide, N-hydroxy-20-mercaptoeicosanamide, (20-mercaptoeicosyl)phosphonic acid, 20-mercaptoeicosan-1-ol, 20-mercaptoeicosanoic acid, 20-mercaptoeicosyl dihydrogen phosphate, 20-mercaptoeicosane(dithioc) acid, (20-mercaptoeicosyl)carbamodithioc acid, 20-hydroxyeicosanoic acid, 20-hydroxyeicosyl dihydrogen phosphate, 20-hydroxyeicosane(dithioc) acid, (20-hydroxyeicosyl)carbamodithioc acid, N,20-dihydroxyeicosanamide, (20-iydroxyeicosyl)phosphonic acid, 20-phosphonoeicosanoic acid, 20-(phosphonooxy)eicosanoic acid, 20-mercapto-20-thioxoeicosanoic acid, 20-(hydroxyamino)-20-oxoeicosanoic acid, 20-[(mercaptocarbonothioyl)amino]eicosanoic acid, [20-(hydroxyamino)-20-oxoeicosyl]phosphonic acid, [20-(phosphonooxy)eicosyl]phosphonic acid, {20-[(mercaptocarbonothioyl)amino]eicosyl}phosphonic acid, 20-phosphonoeicosane(dithioic) acid, [20-(phosphonooxy)eicosyl]carbamodithioic acid, 20-(hydroxyamino)-20-oxoeicosyl dihydrogen phosphate, 20-(phosphonooxy)eicosane(dithioic) acid, 20-(hydroxyamino)-20-oxoeicosane(dithioic) acid, [20-(hydroxyamino)-20-oxoeicosyl]carbamodithioic acid, 20-[(mercaptocarbonothioyl)amino]eicosane(dithioic) acid, 20-aminoeicosylmethyldichlorosilane, 20-mercaptoeicosylmethyldichlorosilane, 20-isocyanatoeicosylmethyldichlorosilane, 20-carboxyeicosylmethyldichlorosilane, 20-hydroxyeicosylmethyldichlorosilane, 20-iodoeicosylmethyldichlorosilane, 20-chloroeicosylmethyldichlorosilane, 20-bromoeicosylmethyldichlorosilane, 20-aminoeicosyltrichlorosilane, 20-mercaptoeicosyltrichlorosilane, 20-isocyanatoeicosyltrichlorosilane, 20-carboxyeicosyltrichlorosilane, 20-hydroxyeicosyltrichlorosilane, 20-iodoeicosyltrichlorosilane, 20-chloroeicosyltrichlorosilane, 20-bromoeicosyltrichlorosilane, 20-aminoeicosylchlorodimethylsilane, 20-mercaptoeicosylchlorodimethylsilane, 20-isocyanatoeicosylchlorodimethylsilane, 20-carboxyeicosylchlorodimethylsilane, 20-hydroxyeicosylchlorodimethylsilane, 20-iodoeicosylchlorodimethylsilane, 20-chloroeicosylchlorodimethylsilane, 20-bromoeicosylchlorodimethylsilane, 20-aminoeicosyltriethoxysilane, 20-mercaptoeicosyltriethoxysilane, 20-isocyanatoeicosyltriethoxysilane, 20-carboxyeicosyltriethoxysilane, 20-hydroxyeicosyltriethoxysilane, 20-iodoeicosyltriethoxysilane, 20-chloroeicosyltriethoxysilane, 20-bromoeicosyltriethoxysilane, 20-aminoeicosyltrimethoxysilane, 20-mercaptoeicosyltrimethoxysilane, 20-isocyanatoeicosyltrimethoxysilane, 20-carboxyeicosyltrimethoxysilane, 20-hydroxyeicosyltrimethoxysilane, 20-iodoeicosyltrimethoxysilane, 20-chloroeicosyltrimethoxysilane, 20-bromoeicosyltrimethoxysilane, 20-bromoeicosan-1-ol, 20-bromoeicosanoic acid, 20-bromoeicosyl dihydrogen phosphate, 20-bromoeicosan-1-thiol, 20-bromoeicosane(dithioc) acid, (20-bromoeicosyl)carbamodithioc acid, (20-bromoeicosyl)phosphonic acid, (20-bromo)-N-hydroxyeicosanamide, 20-chloroeicosan-1-ol, 20-chloroeicosanoic acid, 20-chloroeicosyl dihydrogen phosphate, 20-chloroeicosan-1-thiol, 20-chloroeicosane(dithioc) acid, (20-chloroeicosyl)carbamodithioc acid, (20-chloroeicosyl)phosphonic acid, (20-chloro)-N-hydroxyeicosanamide, 20-iodoeicosan-1-ol, 20-iodoeicosanoic acid, 20-iodoeicosyl dihydrogen phosphate, 20-iodoeicosan-1-thiol, 20-iodoeicosane(dithioc) acid, (20-iodoeicosyl)carbamodithioc acid, (20-iodoeicosyl)phosphonic acid, (20-iodo)-N-hydroxyeicosanamide, 20-isocyanatoeicosan-1-ol, 20-isocyanatoeicosanoic acid, 20-isocyanatoeicosyl dihydrogen phosphate, 20-isocyanatoeicosan-1-thiol, 20-isocyanatoeicosane(dithioc) acid, (20-isocyanatoeicosyl)carbamodithioc acid, (20-isocyanatoeicosyl)phosphonic acid, (20-isocyanato)-N-hydroxyeicosanamide, 21-aminouncosan-1-ol, 21-aminouncosanoic acid, 21-aminouncosyl dihydrogen phosphate, 21-aminouncosan-1-thiol, 21-aminouncosane(dithioc) acid, (21-aminouncosyl)carbamodithioc acid, (21-aminouncosyl)phosphonic acid, (21-amino)-N-hydroxyuncosanamide, N-hydroxy-21-mercaptouncosanamide, (21-mercaptouncosyl)phosphonic acid, 21-mercaptouncosan-1-ol, 21-mercaptouncosanoic acid, 21-mercaptouncosyl dihydrogen phosphate, 21-mercaptouncosane(dithioc) acid, (21-mercaptouncosyl)carbamodithioc acid, 21-hydroxyuncosanoic acid, 21-hydroxyuncosyl dihydrogen phosphate, 21-hydroxyuncosane(dithioc) acid, (21-hydroxyuncosyl)carbamodithioc acid, N,21-dihydroxyuncosanamide, (21-hydroxyuncosyl)phosphonic acid, 21-phosphonouncosanoic acid, 21-(phosphonooxy)uncosanoic acid, 21-mercapto-21-thioxouncosanoic acid, 21-(hydroxyamino)-21-oxouncosanoic acid, 21-[(mercaptocarbonothioyl)amino]uncosanoic acid, [21-(hydroxyamino)-21-oxouncosyl]phosphonic acid, [21-(phosphonooxy)uncosyl]phosphonic acid, {21-[(mercaptocarbonothioyl)amino]uncosyl}phosphonic acid, 21-phosphonouncosane(dithioic) acid, [21-(phosphonooxy)uncosyl]carbamodithioic acid, 21-(hydroxyamino)-21-oxouncosyl dihydrogen phosphate, 21-(phosphonooxy)uncosane(dithioic) acid, 21-(hydroxyamino)-21-oxouncosane(dithioic) acid, [21-(hydroxyamino)-21-oxouncosyl]carbamodithioic acid, 21-[(mercaptocarbonothioyl)amino]uncosane(dithioic) acid, 21-aminouncosylmethyldichlorosilane, 21-mercaptouncosylmethyldichlorosilane, 21-isocyanatouncosylmethyldichlorosilane, 21-carboxyuncosylmethyldichlorosilane, 21-hydroxyuncosylmethyldichlorosilane, 21-iodouncosylmethyldichlorosilane, 21-chlorouncosylmethyldichlorosilane, 21-bromouncosylmethyldichlorosilane, 21-aminouncosyltrichlorosilane, 21-mercaptouncosyltrichlorosilane, 21-isocyanatouncosyltrichlorosilane, 21-carboxyuncosyltrichlorosilane, 21-hydroxyuncosyltrichlorosilane, 21-iodouncosyltrichlorosilane, 21-chlorouncosyltrichlorosilane, 21-bromouncosyltrichlorosilane, 21-aminouncosylchlorodimethylsilane, 21-mercaptouncosylchlorodimethylsilane, 21-isocyanatouncosylchlorodimethylsilane, 21-carboxyuncosylchlorodimethylsilane, 21-hydroxyuncosylchlorodimethylsilane, 21-iodouncosylchlorodimethylsilane, 21-chlorouncosylchlorodimethylsilane, 21-bromouncosylchlorodimethylsilane, 21-aminouncosyltriethoxysilane, 21-mercaptouncosyltriethoxysilane, 21-isocyanatouncosyltriethoxysilane, 21-carboxyuncosyltriethoxysilane, 21-hydroxyuncosyltriethoxysilane, 21-iodouncosyltriethoxysilane, 21-chlorouncosyltriethoxysilane, 21-bromouncosyltriethoxysilane, 21-aminouncosyltrimethoxysilane, 21-mercaptouncosyltrimethoxysilane, 21-isocyanatouncosyltrimethoxysilane, 21-carboxyuncosyltrimethoxysilane, 21-hydroxyuncosyltrimethoxysilane, 21-iodouncosyltrimethoxysilane, 21-chlorouncosyltrimethoxysilane, 21-bromouncosyltrimethoxysilane, 21-bromouncosan-1-ol, 21-bromouncosanoic acid, 21-bromouncosyl dihydrogen phosphate, 21-bromouncosan-1-thiol, 21-bromouncosane(dithioc) acid, (21-bromouncosyl)carbamodithioc acid, (21-bromouncosyl)phosphonic acid, (21-bromo)-N-hydroxyuncosanamide, 21-chlorouncosan-1-ol, 21-chlorouncosanoic acid, 21-chlorouncosyl dihydrogen phosphate, 21-chlorouncosan-1-thiol, 21-chlorouncosane(dithioc) acid, (21-chlorouncosyl)carbamodithioc acid, (21-chlorouncosyl)phosphonic acid, (21-chloro)-N-hydroxyuncosanamide, 21-iodouncosan-1-ol, 21-iodouncosanoic acid, 21-iodouncosyl dihydrogen phosphate, 21-iodouncosan-1-thiol, 21-iodouncosane(dithioc) acid, (21-iodouncosyl)carbamodithioc acid, (21-iodouncosyl)phosphonic acid, (21-iodo)-N-hydroxyuncosanamide, 21-isocyanatouncosan-1-ol, 21-isocyanatouncosanoic acid, 21-isocyanatouncosyl dihydrogen phosphate, 21-isocyanatouncosan-1-thiol, 21-isocyanatouncosane(dithioc) acid, (21-isocyanatouncosyl)carbamodithioc acid, (21-isocyanatouncosyl)phosphonic acid, (21-isocyanato)-N-hydroxyuncosanamide, 22-aminodocosan-1-ol, 22-aminodocosanoic acid, 22-aminodocosyl dihydrogen phosphate, 22-aminodocosan-1-thiol, 22-aminodocosane(dithioc) acid, (22-aminodocosyl)carbamodithioc acid, (22-aminodocosyl)phosphonic acid, (22-amino)-N-hydroxydocosanamide, N-hydroxy-22-mercaptodocosanamide, (22-mercaptodocosyl)phosphonic acid, 22-mercaptodocosan-1-ol, 22-mercaptodocosanoic acid, 22-mercaptodocosyl dihydrogen phosphate, 22-mercaptodocosane(dithioc) acid, (22-mercaptodocosyl)carbamodithioc acid, 22-hydroxydocosanoic acid, 22-hydroxydocosyl dihydrogen phosphate, 22-hydroxydocosane(dithioc) acid, (22-hydroxydocosyl)carbamodithioc acid, N,22-dihydroxydocosanamide, (22-hydroxydocosyl)phosphonic acid, 22-phosphonodocosanoic acid, 22-(phosphonooxy)docosanoic acid, 22-mercapto-22-thioxodocosanoic acid, 22-(hydroxyamino)-22-oxodocosanoic acid, 22-[(mercaptocarbonothioyl)amino]docosanoic acid, [22-(hydroxyamino)-22-oxodocosyl]phosphonic acid, [22-(phosphonooxy)docosyl]phosphonic acid, {22-[(mercaptocarbonothioyl)amino]docosyl}phosphonic acid, 22-phosphonodocosane(dithioic) acid, [22-(phosphonooxy)docosyl]carbamodithioic acid, 22-(hydroxyamino)-22-oxodocosyl dihydrogen phosphate, 22-(phosphonooxy)docosane(dithioic) acid, 22-(hydroxyamino)-22-oxodocosane(dithioic) acid, [22-(hydroxyamino)-22-oxodocosyl]carbamodithioic acid, 22-[(mercaptocarbonothioyl)amino]docosane(dithioic) acid, 22-aminodocosylmethyldichlorosilane, 22-mercaptodocosylmethyldichlorosilane, 22-isocyanatodocosylmethyldichlorosilane, 22-carboxydocosylmethyldichlorosilane, 22-hydroxydocosylmethyldichlorosilane, 22-iododocosylmethyldichlorosilane, 22-chlorodocosylmethyldichlorosilane, 22-bromodocosylmethyldichlorosilane, 22-aminodocosyltrichlorosilane, 22-mercaptodocosyltrichliorosilane, 22-isocyanatodocosyltrichlorosilane, 22-carboxydocosyltrichlorosilane, 22-hydroxydocosyltrichlorosilane, 22-iododocosyltrichlorosilane, 22-chlorodocosyltrichlorosilane, 22-bromodocosyltrichlorosilane, 22-aminodocosylchlorodimethylsilane, 22-mercaptodocosylchlorodimethylsilane, 22-isocyanatodocosylchlorodimethylsilane, 22-carboxydocosylchlorodimethylsilane, 22-hydroxydocosylchlorodimethylsilane, 22-iododocosylchlorodimethylsilane, 22-chlorodocosylchlorodimethylsilane, 22-bromodocosylchlorodimethylsilane, 22-aminodocosyltriethoxysilane, 22-mercaptodocosyltriethoxysilane, 22-isocyanatodocosyltriethoxysilane, 22-carboxydocosyltriethoxysilane, 22-hydroxydocosyltriethoxysilane, 22-iododocosyltriethoxysilane, 22-chlorodocosyltriethoxysilane, 22-bromodocosyltriethoxysilane, 22-aminodocosyltrimethoxysilane, 22-mercaptodocosyltrimethoxysilane, 22-isocyanatodocosyltrimethoxysilane, 22-carboxydocosyltrimethoxysilane, 22-hydroxydocosyltrimethoxysilane, 22-iododocosyltrimethoxysilane, 22-chlorodocosyltrimethoxysilane, 22-bromodocosyltrimethoxysilane, 22-bromodocosan-1-ol, 22-bromodocosanoic acid, 22-bromodocosyl dihydrogen phosphate, 22-bromodocosan-1-thiol, 22-bromodocosane(dithioc) acid, (22-bromodocosyl)carbamodithioc acid, (22-bromodocosyl)phosphonic acid, (22-bromo)-N-hydroxydocosanamide, 22-chlorodocosan-1-ol, 22-chlorodocosanoic acid, 22-chlorodocosyl dihydrogen phosphate, 22-chlorodocosan-1-thiol, 22-chlorodocosane(dithioc) acid, (22-chlorodocosyl)carbamodithioc acid, (22-chlorodocosyl)phosphonic acid, (22-chloro)-N-hydroxydocosanamide, 22-iododocosan-1-ol, 22-iododocosanoic acid, 22-iododocosyl dihydrogen phosphate, 22-iododocosan-1-thiol, 22-iododocosane(dithioc) acid, (22-iododocosyl)carbamodithioc acid, (22-iododocosyl)phosphonic acid, (22-iodo)-N-hydroxydocosanamide, 22-isocyanatodocosan-1-ol, 22-isocyanatodocosanoic acid, 22-isocyanatodocosyl dihydrogen phosphate, 22-isocyanatodocosan-1-thiol, 22-isocyanatodocosane(dithioc) acid, (22-isocyanatodocosyl)carbamodithioc acid, (22-isocyanatodocosyl)phosphonic acid, (22-isocyanato)-N-hydroxydocosanamide, 23-aminotricosan-1-ol, 23-aminotricosanoic acid, 23-aminotricosyl dihydrogen phosphate, 23-aminotricosan-1-thiol, 23-aminotricosane(dithioc) acid, (23-aminotricosyl)carbamodithioc acid, (23-aminotricosyl)phosphonic acid, (23-amino)-N-hydroxytricosanamide, N-hydroxy-23-mercaptotricosanamide, (23-mercaptotricosyl)phosphonic acid, 23-mercaptotricosan-1-ol, 23-mercaptotricosanoic acid, 23-mercaptotricosyl dihydrogen phosphate, 23-mercaptotricosane(dithioc) acid, (23-mercaptotricosyl)carbamodithioc acid, 23-hydroxytricosanoic acid, 23-hydroxytricosyl dihydrogen phosphate, 23-hydroxytricosane(dithioc) acid, (23-hydroxytricosyl)carbamodithioc acid, N,23-dihydroxytricosanamide, (23-hydroxytricosyl)phosphonic acid, 23-phosphonotricosanoic acid, 23-(phosphonooxy)tricosanoic acid, 23-mercapto-23-thioxotricosanoic acid, 23-(hydroxyamino)-23-oxotricosanoic acid, 23-[(mercaptocarbonothioyl)amino]tricosanoic acid, [23-(hydroxyamino)-23-oxotricosyl]phosphonic acid, [23-(phosphonooxy)tricosyl]phosphonic acid, {23-[(mercaptocarbonothioyl)amino]tricosyl}phosphonic acid, 23-phosphonotricosane(dithioic) acid, [23-(phosphonooxy)tricosyl]carbamodithioic acid, 23-(hydroxyamino)-23-oxotricosyl dihydrogen phosphate, 23-(phosphonooxy)tricosane(dithioic) acid, 23-(hydroxyamino)-23-oxotricosane(dithioic) acid, [23-(hydroxyamino)-23-oxotricosyl]carbamodithioic acid, 23-[(mercaptocarbonothioyl)amino]tricosane(dithioic) acid, 23-aminotricosylmethyldichlorosilane, 23-mercaptotricosylmethyldichlorosilane, 23-isocyanatotricosylmethyldichlorosilane, 23-carboxytricosylmethyldichlorosilane, 23-hydroxytricosylmethyldichlorosilane, 23-iodotricosylmethyldichlorosilane, 23-chlorotricosylmethyldichlorosilane, 23-bromotricosylmethyldichlorosilane, 23-aminotricosyltrichlorosilane, 23-mercaptotricosyltrichlorosilane, 23-isocyanatotricosyltrichlorosilane, 23-carboxytricosyltrichlorosilane, 23-hydroxytricosyltrichlorosilane, 23-iodotricosyltrichlorosilane, 23-chlorotricosyltrichlorosilane, 23-bromotricosyltrichlorosilane, 23-aminotricosylchlorodimethylsilane, 23-mercaptotricosylchlorodimethylsilane, 23-isocyanatotricosylchlorodimethylsilane, 23-carboxytricosylchlorodimethylsilane, 23-hydroxytricosylchlorodimethylsilane, 23-iodotricosylchlorodimethylsilane, 23-chlorotricosylchlorodimethylsilane, 23-bromotricosylchlorodimethylsilane, 23-aminotricosyltriethoxysilane, 23-mercaptotricosyltriethoxysilane, 23-isocyanatotricosyltriethoxysilane, 23-carboxytricosyltriethoxysilane, 23-hydroxytricosyltriethoxysilane, 23-iodotricosyltriethoxysilane, 23-chlorotricosyltriethoxysilane, 23-bromotricosyltriethoxysilane, 23-aminotricosyltrimethoxysilane, 23-mercaptotricosyltrimethoxysilane, 23-isocyanatotricosyltrimethoxysilane, 23-carboxytricosyltrimethoxysilane, 23-hydroxytricosyltrimethoxysilane, 23-iodotricosyltrimethoxysilane, 23-chlorotricosyltrimethoxysilane, 23-bromotricosyltrimethoxysilane, 23-bromotricosan-1-ol, 23-bromotricosanoic acid, 23-bromotricosyl dihydrogen phosphate, 23-bromotricosan-1-thiol, 23-bromotricosane(dithioc) acid, (23-bromotricosyl)carbamodithioc acid, (23-bromotricosyl)phosphonic acid, (23-bromo)-N-hydroxytricosanamide, 23-chlorotricosan-1-ol, 23-chlorotricosanoic acid, 23-chlorotricosyl dihydrogen phosphate, 23-chlorotricosan-1-thiol, 23-chlorotricosane(dithioc) acid, (23-chlorotricosyl)carbamodithioc acid, (23-chlorotricosyl)phosphonic acid, (23-chloro)-N-hydroxytricosanamide, 23-iodotricosan-1-ol, 23-iodotricosanoic acid, 23-iodotricosyl dihydrogen phosphate, 23-iodotricosan-1-thiol, 23-iodotricosane(dithioc) acid, (23-iodotricosyl)carbamodithioc acid, (23-iodotricosyl)phosphonic acid, (23-iodo)-N-hydroxytricosanamide, 23-isocyanatotricosan-1-ol, 23-isocyanatotricosanoic acid, 23-isocyanatotricosyl dihydrogen phosphate, 23-isocyanatotricosan-1-thiol, 23-isocyanatotricosane(dithioc) acid, (23-isocyanatotricosyl)carbamodithioc acid, (23-isocyanatotricosyl)phosphonic acid, (23-isocyanato)-N-hydroxytricosanamide, 24-aminotetracosan-1-ol, 24-aminotetracosanoic acid, 24-aminotetracosyl dihydrogen phosphate, 24-aminotetracosan-1-thiol, 24-aminotetracosane(dithioc) acid, (24-aminotetracosyl)carbamodithioc acid, (24-aminotetracosyl)phosphonic acid, (24-amino)-N-hydroxytetracosanamide, N-hydroxy-24-mercaptotetracosanamide, (24-mercaptotetracosyl)phosphonic acid, 24-mercaptotetracosan-1-ol, 24-mercaptotetracosanoic acid, 24-mercaptotetracosyl dihydrogen phosphate, 24-mercaptotetracosane(dithioc) acid, (24-mercaptotetracosyl)carbamodithioc acid, 24-hydroxytetracosanoic acid, 24-hydroxytetracosyl dihydrogen phosphate, 24-hydroxytetracosane(dithioc) acid, (24-hydroxytetracosyl)carbamodithioc acid, N,24-dihydroxytetracosanamide, (24-hydroxytetracosyl)phosphonic acid, 24-phosphonotetracosanoic acid, 24-(phosphonooxy)tetracosanoic acid, 24-mercapto-24-thioxotetracosanoic acid, 24-(hydroxyamino)-24-oxotetracosanoic acid, 24-[(mercaptocarbonothioyl)amino]tetracosanoic acid, [24-(hydroxyamino)-24-oxotetracosyl]phosphonic acid, [24-(phosphonooxy)tetracosyl]phosphonic acid, {24-[(mercaptocarbonothioyl)amino]tetracosyl}phosphonic acid, 24-phosphonotetracosane(dithioic) acid, [24-(phosphonooxy)tetracosyl]carbamodithioic acid, 24-(hydroxyamino)-24-oxotetracosyl dihydrogen phosphate, 24-(phosphonooxy)tetracosane(dithioic) acid, 24-(hydroxyamino)-24-oxotetracosane(dithioic) acid, [24-(hydroxyamino)-24-oxotetracosyl]carbamodithioic acid, 24-[(mercaptocarbonothioyl)amino]tetracosane(dithioic) acid, 24-aminotetracosylmethyldichlorosilane, 24-mercaptotetracosylmethyldichlorosilane, 24-isocyanatotetracosylmethyldichlorosilane, 24-carboxytetracosylmethyldichlorosilane, 24-hydroxytetracosylmethyldichlorosilane, 24-iodotetracosylmethyldichlorosilane, 24-chlorotetracosylmethyldichlorosilane, 24-bromotetracosylmethyldichlorosilane, 24-aminotetracosyltrichlorosilane, 24-mercaptotetracosyltrichlorosilane, 24-isocyanatotetracosyltrichlorosilane, 24-carboxytetracosyltrichliorosilane, 24-hydroxytetracosyltrichlorosilane, 24-iodotetracosyltrichlorosilane, 24-chlorotetracosyltrichlorosilane, 24-bromotetracosyltrichlorosilane, 24-aminotetracosylchlorodimethylsilane, 24-mercaptotetracosylchlorodimethylsilane, 24-isocyanatotetracosylchlorodimethylsilane, 24-carboxytetracosylchlorodimethylsilane, 24-hydroxytetracosylchlorodimethylsilane, 24-iodotetracosylchlorodimethylsilane, 24-chlorotetracosylchlorodimethylsilane, 24-bromotetracosylchlorodimethylsilane, 24-aminotetracosyltriethoxysilane, 24-mercaptotetracosyltriethoxysilane, 24-isocyanatotetracosyltriethoxysilane, 24-carboxytetracosyltriethoxysilane, 24-hydroxytetracosyltriethoxysilane, 24-iodotetracosyltriethoxysilane, 24-chlorotetracosyltriethoxysilane, 24-bromotetracosyltriethoxysilane, 24-aminotetracosyltrimethoxysilane, 24-mercaptotetracosyltrimethoxysilane, 24-isocyanatotetracosyltrimethoxysilane, 24-carboxytetracosyltrimethoxysilane, 24-hydroxytetracosyltrimethoxysilane, 24-iodotetracosyltrimethoxysilane, 24-chlorotetracosyltrimethoxysilane, 24-bromotetracosyltrimethoxysilane, 24-bromotetracosan-1-ol, 24-bromotetracosanoic acid, 24-bromotetracosyl dihydrogen phosphate, 24-bromotetracosan-1-thiol, 24-bromotetracosane(dithioc) acid, (24-bromotetracosyl)carbamodithioc acid, (24-bromotetracosyl)phosphonic acid, (24-bromo)-N-hydroxytetracosanamide, 24-chlorotetracosan-1-ol, 24-chlorotetracosanoic acid, 24-chlorotetracosyl dihydrogen phosphate, 24-chlorotetracosan-1-thiol, 24-chlorotetracosane(dithioc) acid, (24-chlorotetracosyl)carbamodithioc acid, (24-chlorotetracosyl)phosphonic acid, (24-chloro)-N-hydroxytetracosanamide, 24-iodotetracosan-1-ol, 24-iodotetracosanoic acid, 24-iodotetracosyl dihydrogen phosphate, 24-iodotetracosan-1-thiol, 24-iodotetracosane(dithioc) acid, (24-iodotetracosyl)carbamodithioc acid, (24-iodotetracosyl)phosphonic acid, (24-iodo)-N-hydroxytetracosanamide, 24-isocyanatotetracosan-1-ol, 24-isocyanatotetracosanoic acid, 24-isocyanatotetracosyl dihydrogen phosphate, 24-isocyanatotetracosan-1-thiol, 24-isocyanatotetracosane(dithioc) acid, (24-isocyanatotetracosyl)carbamodithioc acid, (24-isocyanatotetracosyl)phosphonic acid, (24-isocyanato)-N-hydroxytetracosanamide, 25-aminopentacosan-1-ol, 25-aminopentacosanoic acid, 25-aminopentacosyl dihydrogen phosphate, 25-aminopentacosan-1-thiol, 25-aminopentacosane(dithioc) acid, (25-aminopentacosyl)carbamodithioc acid, (25-aminopentacosyl)phosphonic acid, (25-amino)-N-hydroxypentacosanamide, N-hydroxy-25-mercaptopentacosanamide, (25-mercaptopentacosyl)phosphonic acid, 25-mercaptopentacosan-1-ol, 25-mercaptopentacosanoic acid, 25-mercaptopentacosyl dihydrogen phosphate, 25-mercaptopentacosane(dithioc) acid, (25-mercaptopentacosyl)carbamodithioc acid, 25-hydroxypentacosanoic acid, 25-hydroxypentacosyl dihydrogen phosphate, 25-hydroxypentacosane(dithioc) acid, (25-hydroxypentacosyl)carbamodithioc acid, N,25-dihydroxypentacosanamide, (25-hydroxypentacosyl)phosphonic acid, 25-phosphonopentacosanoic acid, 25-(phosphonooxy)pentacosanoic acid, 25-mercapto-25-thioxopentacosanoic acid, 25-(hydroxyamino)-25-oxopentacosanoic acid, 25-[(mercaptocarbonothioyl)amino]pentacosanoic acid, [25-(hydroxyamino)-25-oxopentacosyl]phosphonic acid, [25-(phosphonooxy)pentacosyl]phosphonic acid, {25-[(mercaptocarbonothioyl)amino]pentacosyl}phosphonic acid, 25-phosphonopentacosane(dithioic) acid, [25-(phosphonooxy)pentacosyl]carbamodithioic acid, 25-(hydroxyamino)-25-oxopentacosyl dihydrogen phosphate, 25-(phosphonooxy)pentacosane(dithioic) acid, 25-(hydroxyamino)-25-oxopentacosane(dithioic) acid, [25-(hydroxyamino)-25-oxopentacosyl]carbamodithioic acid, 25-[(mercaptocarbonothioyl)amino]pentacosane(dithioic) acid, 25-aminopentacosylmethyldichlorosilane, 25-mercaptopentacosylmethyldichlorosilane, 25-isocyanatopentacosylmethyldichlorosilane, 25-carboxypentacosylmethyldichlorosilane, 25-hydroxypentacosylmethyldichlorosilane, 25-iodopentacosylmethyldichlorosilane, 25-chloropentacosylmethyldichlorosilane, 25-bromopentacosylmethyldichlorosilane, 25-aminopentacosyltrichlorosilane, 25-mercaptopentacosyltrichlorosilane, 25-isocyanatopentacosyltrichlorosilane, 25-carboxypentacosyltrichlorosilane, 25-hydroxypentacosyltrichlorosilane, 25-iodopentacosyltrichlorosilane, 25-chloropentacosyltrichlorosilane, 25-bromopentacosyltrichlorosilane, 25-aminopentacosylchlorodimethylsilane, 25-mercaptopentacosylchlorodimethylsilane, 25-isocyanatopentacosylchlorodimethylsilane, 25-carboxypentacosylchlorodimethylsilane, 25-hydroxypentacosylchlorodimethylsilane, 25-iodopentacosylchlorodimethylsilane, 25-chloropentacosylchlorodimethylsilane, 25-bromopentacosylchlorodimethylsilane, 25-aminopentacosyltriethoxysilane, 25-mercaptopentacosyltriethoxysilane, 25-isocyanatopentacosyltriethoxysilane, 25-carboxypentacosyltriethoxysilane, 25-hydroxypentacosyltriethoxysilane, 25-iodopentacosyltriethoxysilane, 25-chloropentacosyltriethoxysilane, 25-bromopentacosyltriethoxysilane, 25-aminopentacosyltrimethoxysilane, 25-mercaptopentacosyltrimethoxysilane, 25-isocyanatopentacosyltrimethoxysilane, 25-carboxypentacosyltrimethoxysilane, 25-hydroxypentacosyltrimethoxysilane, 25-iodopentacosyltrimethoxysilane, 25-chloropentacosyltrimethoxysilane, 25-bromopentacosyltrimethoxysilane, 25-bromopentacosan-1-ol, 25-bromopentacosanoic acid, 25-bromopentacosyl dihydrogen phosphate, 25-bromopentacosan-1-thiol, 25-bromopentacosane(dithioc) acid, (25-bromopentacosyl)carbamodithioc acid, (25-bromopentacosyl)phosphonic acid, (25-bromo)-N-hydroxypentacosanamide, 25-chloropentacosan-1-ol, 25-chloropentacosanoic acid, 25-chloropentacosyl dihydrogen phosphate, 25-chloropentacosan-1-thiol, 25-chloropentacosane(dithioc) acid, (25-chloropentacosyl)carbamodithioc acid, (25-chloropentacosyl)phosphonic acid, (25-chloro)-N-hydroxypentacosanamide, 25-iodopentacosan-1-ol, 25-iodopentacosanoic acid, 25-iodopentacosyl dihydrogen phosphate, 25-iodopentacosan-1-thiol, 25-iodopentacosane(dithioc) acid, (25-iodopentacosyl)carbamodithioc acid, (25-iodopentacosyl)phosphonic acid, (25-iodo)-N-hydroxypentacosanamide, 25-isocyanatopentacosan-1-ol, 25-isocyanatopentacosanoic acid, 25-isocyanatopentacosyl dihydrogen phosphate, 25-isocyanatopentacosan-1-thiol, 25-isocyanatopentacosane(dithioc) acid, (25-isocyanatopentacosyl)carbamodithioc acid, (25-isocyanatopentacosyl)phosphonic acid, (25-isocyanato)-N-hydroxypentacosanamide, 26-aminohexacosan-1-ol, 26-aminohexacosanoic acid, 26-aminohexacosyl dihydrogen phosphate, 26-aminohexacosan-1-thiol, 26-aminohexacosane(dithioc) acid, (26-aminohexacosyl)carbamodithioc acid, (26-aminohexacosyl)phosphonic acid, (26-amino)-N-hydroxyhexacosanamide, N-hydroxy-26-mercaptohexacosanamide, (26-mercaptohexacosyl)phosphonic acid, 26-mercaptohexacosan-1-ol, 26-mercaptohexacosanoic acid, 26-mercaptohexacosyl dihydrogen phosphate, 26-mercaptohexacosane(dithioc) acid, (26-mercaptohexacosyl)carbamodithioc acid, 26-hydroxyhexacosanoic acid, 26-hydroxyhexacosyl dihydrogen phosphate, 26-hydroxyhexacosane(dithioc) acid, (26-hydroxyhexacosyl)carbamodithioc acid, N,26-dihydroxyhexacosanamide, (26-hydroxyhexacosyl)phosphonic acid, 26-phosphonohexacosanoic acid, 26-(phosphonooxy)hexacosanoic acid, 26-mercapto-26-thioxohexacosanoic acid, 26-(hydroxyamino)-26-oxohexacosanoic acid, 26-[(mercaptocarbonothioyl)amino]hexacosanoic acid, [26-(hydroxyamino)-26-oxohexacosyl]phosphonic acid, [26-(phosphonooxy)hexacosyl]phosphonic acid, {26-[(mercaptocarbonothioyl)amino]hexacosyl}phosphonic acid, 26-phosphonohexacosane(dithioic) acid, [26-(phosphonooxy)hexacosyl]carbamodithioic acid, 26-(hydroxyamino)-26-oxohexacosyl dihydrogen phosphate, 26-(phosphonooxy)hexacosane(dithioic) acid, 26-(hydroxyamino)-26-oxohexacosane(dithioic) acid, [26-(hydroxyamino)-26-oxohexacosyl]carbamodithioic acid, 26-[(mercaptocarbonothioyl)amino]hexacosane(dithioic) acid, 26-aminohexacosylmethyldichlorosilane, 26-mercaptohexacosylmethyldichlorosilane, 26-isocyanatohexacosylmethyldichlorosilane, 26-carboxyhexacosylmethyldichlorosilane, 26-hydroxyhexacosylmethyldichlorosilane, 26-iodohexacosylmethyldichlorosilane, 26-chlorohexacosylmethyldichlorosilane, 26-bromohexacosylmethyldichlorosilane, 26-aminohexacosyltrichlorosilane, 26-mercaptohexacosyltrichlorosilane, 26-isocyanatohexacosyltrichlorosilane, 26-carboxyhexacosyltrichlorosilane, 26-hydroxyhexacosyltrichlorosilane, 26-iodohexacosyltrichlorosilane, 26-chlorohexacosyltrichlorosilane, 26-bromohexacosyltrichlorosilane, 26-aminohexacosylchlorodimethylsilane, 26-mercaptohexacosylchlorodimethylsilane, 26-isocyanatohexacosylchlorodimethylsilane, 26-carboxyhexacosylchlorodimethylsilane, 26-hydroxyhexacosylchlorodimethylsilane, 26-iodohexacosylchlorodimethylsilane, 26-chlorohexacosylchlorodimethylsilane, 26-bromohexacosylchlorodimethylsilane, 26-aminohexacosyltriethoxysilane, 26-mercaptohexacosyltriethoxysilane, 26-isocyanatohexacosyltriethoxysilane, 26-carboxyhexacosyltriethoxysilane, 26-hydroxyhexacosyltriethoxysilane, 26-iodohexacosyltriethoxysilane, 26-chlorohexacosyltriethoxysilane, 26-bromohexacosyltriethoxysilane, 26-aminohexacosyltrimethoxysilane, 26-mercaptohexacosyltrimethoxysilane, 26-isocyanatohexacosyltrimethoxysilane, 26-carboxyhexacosyltrimethoxysilane, 26-hydroxyhexacosyltrimethoxysilane, 26-iodohexacosyltrimethoxysilane, 26-chlorohexacosyltrimethoxysilane, 26-bromohexacosyltrimethoxysilane, 26-bromohexacosan-1-ol, 26-bromohexacosanoic acid, 26-bromohexacosyl dihydrogen phosphate, 26-bromohexacosan-1-thiol, 26-bromohexacosane(dithioc) acid, (26-bromohexacosyl)carbamodithioc acid, (26-bromohexacosyl)phosphonic acid, (26-bromo)-N-hydroxyhexacosanamide, 26-chlorohexacosan-1-ol, 26-chlorohexacosanoic acid, 26-chlorohexacosyl dihydrogen phosphate, 26-chlorohexacosan-1-thiol, 26-chlorohexacosane(dithioc) acid, (26-chlorohexacosyl)carbamodithioc acid, (26-chlorohexacosyl)phosphonic acid, (26-chloro)-N-hydroxyhexacosanamide, 26-iodohexacosan-1-ol, 26-iodohexacosanoic acid, 26-iodohexacosyl dihydrogen phosphate, 26-iodohexacosan-1-thiol, 26-iodohexacosane(dithioc) acid, (26-iodohexacosyl)carbamodithioc acid, (26-iodohexacosyl)phosphonic acid, (26-iodo)-N-hydroxyhexacosanamide, 26-isocyanatohexacosan-1-ol, 26-isocyanatohexacosanoic acid, 26-isocyanatohexacosyl dihydrogen phosphate, 26-isocyanatohexacosan-1-thiol, 26-isocyanatohexacosane(dithioc) acid, (26-isocyanatohexacosyl)carbamodithioc acid, (26-isocyanatohexacosyl)phosphonic acid, (26-isocyanato)-N-hydroxyhexacosanamide, 27-aminoheptacosan-1-ol, 27-aminoheptacosanoic acid, 27-aminoheptacosyl dihydrogen phosphate, 27-aminoheptacosan-1-thiol, 27-aminoheptacosane(dithioc) acid, (27-aminoheptacosyl)carbamodithioc acid, (27-aminoheptacosyl)phosphonic acid, (27-amino)-N-hydroxyheptacosanamide, N-hydroxy-27-mercaptoheptacosanamide, (27-mercaptoheptacosyl)phosphonic acid, 27-mercaptoheptacosan-1-ol, 27-mercaptoheptacosanoic acid, 27-mercaptoheptacosyl dihydrogen phosphate, 27-mercaptoheptacosane(dithioc) acid, (27-mercaptoheptacosyl)carbamodithioc acid, 27-hydroxyheptacosanoic acid, 27-hydroxyheptacosyl dihydrogen phosphate, 27-hydroxyheptacosane(dithioc) acid, (27-hydroxyheptacosyl)carbamodithioc acid, N,27-dihydroxyheptacosanamide, (27-hydroxyheptacosyl)phosphonic acid, 27-phosphonoheptacosanoic acid, 27-(phosphonooxy)heptacosanoic acid, 27-mercapto-27-thioxoheptacosanoic acid, 27-(hydroxyamino)-27-oxoheptacosanoic acid, 27-[(mercaptocarbonothioyl)amino]heptacosanoic acid, [27-(hydroxyamino)-27-oxoheptacosyl]phosphonic acid, [27-(phosphonooxy)heptacosyl]phosphonic acid, {27-[(mercaptocarbonothioyl)amino]heptacosyl}phosphonic acid, 27-phosphonoheptacosane(dithioic) acid, [27-(phosphonooxy)heptacosyl]carbamodithioic acid, 27-(hydroxyamino)-27-oxoheptacosyl dihydrogen phosphate, 27-(phosphonooxy)heptacosane(dithioic) acid, 27-(hydroxyamino)-27-oxoheptacosane(dithioic) acid, [27-hydroxyamino)-27-oxoheptacosyl]carbamodithioic acid, 27-[(mercaptocarbonothioyl)amino]heptacosane(dithioic) acid, 27-aminoheptacosylmethyldichlorosilane, 27-mercaptoheptacosylmethyldichlorosilane, 27-isocyanatoheptacosylmethyldichlorosilane, 27-carboxyheptacosylmethyldichlorosilane, 27-hydroxyheptacosylmethyldichlorosilane, 27-iodoheptacosylmethyldichlorosilane, 27-chloroheptacosylmethyldichlorosilane, 27-bromoheptacosylmethyldichlorosilane, 27-aminoheptacosyltrichlorosilane, 27-mercaptoheptacosyltrichlorosilane, 27-isocyanatoheptacosyltrichlorosilane, 27-carboxyheptacosyltrichlorosilane, 27-hydroxyheptacosyltrichlorosilane, 27-iodoheptacosyltrichlorosilane, 27-chloroheptacosyltrichlorosilane, 27-bromoheptacosyltrichlorosilane, 27-aminoheptacosylchlorodimethylsilane, 27-mercaptoheptacosylchlorodimethylsilane, 27-isocyanatoheptacosylchlorodimethylsilane, 27-carboxyheptacosylchlorodimethylsilane, 27-hydroxyheptacosylchlorodimethylsilane, 27-iodoheptacosylchlorodimethylsilane, 27-chloroheptacosylchlorodimethylsilane, 27-bromoheptacosylchlorodimethylsilane, 27-aminoheptacosyltriethoxysilane, 27-mercaptoheptacosyltriethoxysilane, 27-isocyanatoheptacosyltriethoxysilane, 27-carboxyheptacosyltriethoxysilane, 27-hydroxyheptacosyltriethoxysilane, 27-iodoheptacosyltriethoxysilane, 27-chloroheptacosyltriethoxysilane, 27-bromoheptacosyltriethoxysilane, 27-aminoheptacosyltrimethoxysilane, 27-mercaptoheptacosyltrimethoxysilane, 27-isocyanatoheptacosyltrimethoxysilane, 27-carboxyheptacosyltrimethoxysilane, 27-hydroxyheptacosyltrimethoxysilane, 27-iodoheptacosyltrimethoxysilane, 27-chloroheptacosyltrimethoxysilane, 27-bromoheptacosyltrimethoxysilane, 27-bromoheptacosan-1-ol, 27-bromoheptacosanoic acid, 27-bromoheptacosyl dihydrogen phosphate, 27-bromoheptacosan-1-thiol, 27-bromoheptacosane(dithioc) acid, (27-bromoheptacosyl)carbamodithioc acid, (27-bromoheptacosyl)phosphonic acid, (27-bromo)-N-hydroxyheptacosanamide, 27-chloroheptacosan-1-ol, 27-chloroheptacosanoic acid, 27-chloroheptacosyl dihydrogen phosphate, 27-chloroheptacosan-1-thiol, 27-chloroheptacosane(dithioc) acid, (27-chloroheptacosyl)carbamodithioc acid, (27-chloroheptacosyl)phosphonic acid, (27-chloro)-N-hydroxyheptacosanamide, 27-iodoheptacosan-1-ol, 27-iodoheptacosanoic acid, 27-iodoheptacosyl dihydrogen phosphate, 27-iodoheptacosan-1-thiol, 27-iodoheptacosane(dithioc) acid, (27-iodoheptacosyl)carbamodithioc acid, (27-iodoheptacosyl)phosphonic acid, (27-iodo)-N-hydroxyheptacosanamide, 27-isocyanatoheptacosan-1-ol, 27-isocyanatoheptacosanoic acid, 27-isocyanatoheptacosyl dihydrogen phosphate, 27-isocyanatoheptacosan-1-thiol, 27-isocyanatoheptacosane(dithioc) acid, (27-isocyanatoheptacosyl)carbamodithioc acid, (27-isocyanatoheptacosyl)phosphonic acid, (27-isocyanato)-N-hydroxyheptacosanamide, 28-aminooctacosan-1-ol, 28-aminooctacosanoic acid, 28-aminooctacosyl dihydrogen phosphate, 28-aminooctacosan-1-thiol, 28-aminooctacosane(dithioc) acid, (28-aminooctacosyl)carbamodithioc acid, (28-aminooctacosyl)phosphonic acid, (28-amino)-N-hydroxyoctacosanamide, N-hydroxy-28-mercaptooctacosanamide, (28-mercaptooctacosyl)phosphonic acid, 28-mercaptooctacosan-1-ol, 28-mercaptooctacosanoic acid, 28-mercaptooctacosyl dihydrogen phosphate, 28-mercaptooctacosane(dithioc) acid, (28-mercaptooctacosyl)carbamodithioc acid, 28-hydroxyoctacosanoic acid, 28-hydroxyoctacosyl dihydrogen phosphate, 28-hydroxyoctacosane(dithioc) acid, (28-hydroxyoctacosyl)carbamodithioc acid, N,28-dihydroxyoctacosanamide, (28-hydroxyoctacosyl)phosphonic acid, 28-phosphonooctacosanoic acid, 28-(phosphonooxy)octacosanoic acid, 28-mercapto-28-thioxooctacosanoic acid, 28-(hydroxyamino)-28-oxooctacosanoic acid, 28-[(mercaptocarbonothioyl)amino]octacosanoic acid, [28-(hydroxyamino)-28-oxooctacosyl]phosphonic acid, [28-(phosphonooxy)octacosyl]phosphonic acid, {28-[(mercaptocarbonothioyl)amino]octacosyl}phosphonic acid, 28-phosphonooctacosane(dithioic) acid, [28-(phosphonooxy)octacosyl]carbamodithioic acid, 28-(hydroxyamino)-28-oxooctacosyl dihydrogen phosphate, 28-(phosphonooxy)octacosane(dithioic) acid, 28-(hydroxyamino)-28-oxooctacosane(dithioic) acid, [28-(hydroxyamino)-28-oxooctacosyl]carbamodithioic acid, 28[(mercaptocarbonothioyl)amino]octacosane(dithioic) acid, 28-aminooctacosylmethyldichlorosilane, 28-mercaptooctacosylmethyldichlorosilane, 28-isocyanatooctacosylmethyldichlorosilane, 28-carboxyoctacosylmethyldichlorosilane, 28-hydroxyoctacosylmethyldichlorosilane, 28-iodooctacosylmethyldichlorosilane, 28-chlorooctacosylmethyldichlorosilane, 28-bromooctacosylmethyldichlorosilane, 28-aminooctacosyltrichlorosilane, 28-mercaptooctacosyltrichlorosilane, 28-isocyanatooctacosyltrichlorosilane, 28-carboxyoctacosyltrichlorosilane, 28-hydroxyoctacosyltrichlorosilane, 28-iodooctacosyltrichlorosilane, 28-chlorooctacosyltrichlorosilane, 28-bromooctacosyltrichlorosilane, 28-aminooctacosylchlorodimethylsilane, 28-mercaptooctacosylchlorodimethylsilane, 28-isocyanatooctacosylchlorodimethylsilane, 28-carboxyoctacosylchlorodimethylsilane, 28-hydroxyoctacosylchlorodimethylsilane, 28-iodooctacosylchlorodimethylsilane, 28-chlorooctacosylchlorodimethylsilane, 28-bromooctacosylchlorodimethylsilane, 28-aminooctacosyltriethoxysilane, 28-mercaptooctacosyltriethoxysilane, 28-isocyanatooctacosyltriethoxysilane, 28-carboxyoctacosyltriethoxysilane, 28-hydroxyoctacosyltriethoxysilane, 28-iodooctacosyltriethoxysilane, 28-chlorooctacosyltriethoxysilane, 28-bromooctacosyltriethoxysilane, 28-aminooctacosyltrimethoxysilane, 28-mercaptooctacosyltrimethoxysilane, 28-isocyanatooctacosyltrimethoxysilane, 28-carboxyoctacosyltrimethoxysilane, 28-hydroxyoctacosyltrimethoxysilane, 28-iodooctacosyltrimethoxysilane, 28-chlorooctacosyltrimethoxysilane, 28-bromooctacosyltrimethoxysilane, 28-bromooctacosan-1-ol, 28-bromooctacosanoic acid, 28-bromooctacosyl dihydrogen phosphate, 28-bromooctacosan-1-thiol, 28-bromooctacosane(dithioc) acid, (28-bromooctacosyl)carbamodithioc acid, (28-bromooctacosyl)phosphonic acid, (28-bromo)-N-hydroxyoctacosanamide, 28-chlorooctacosan-1-ol, 28-chlorooctacosanoic acid, 28-chlorooctacosyl dihydrogen phosphate, 28-chlorooctacosan-1-thiol, 28-chlorooctacosane(dithioc) acid, (28-chlorooctacosyl)carbamodithioc acid, (28-chlorooctacosyl)phosphonic acid, (28-chloro)-N-hydroxyoctacosanamide, 28-iodooctacosan-1-ol, 28-iodooctacosanoic acid, 28-iodooctacosyl dihydrogen phosphate, 28-iodooctacosan-1-thiol, 28-iodooctacosane(dithioc) acid, (28-iodooctacosyl)carbamodithioc acid, (28-iodooctacosyl)phosphonic acid, (28-iodo)-N-hydroxyoctacosanamide, 28-isocyanatooctacosan-1-ol, 28-isocyanatooctacosanoic acid, 28-isocyanatooctacosyl dihydrogen phosphate, 28-isocyanatooctacosan-1-thiol, 28-isocyanatooctacosane(dithioc) acid, (28-isocyanatooctacosyl)carbamodithioc acid, (28-isocyanatooctacosyl)phosphonic acid, (28-isocyanato)-N-hydroxyoctacosanamide, 29-aminononacosan-1-ol, 29-aminononacosanoic acid, 29-aminononacosyl dihydrogen phosphate, 29-aminononacosan-1-thiol, 29-aminononacosane(dithioc) acid, (29-aminononacosyl)carbamodithioc acid, (29-aminononacosyl)phosphonic acid, (29-amino)-N-hydroxynonacosanamide, N-hydroxy-29-mercaptononacosanamide, (29-mercaptononacosyl)phosphonic acid, 29-mercaptononacosan-1-ol, 29-mercaptononacosanoic acid, 29-mercaptononacosyl dihydrogen phosphate, 29-mercaptononacosane(dithioc) acid, (29-mercaptononacosyl)carbamodithioc acid, 29-hydroxynonacosanoic acid, 29-hydroxynonacosyl dihydrogen phosphate, 29-hydroxynonacosane(dithioc) acid, (29-hydroxynonacosyl)carbamodithioc acid, N,29-dihydroxynonacosanamide, (29-hydroxynonacosyl)phosphonic acid, 29-phosphonononacosanoic acid, 29-(phosphonooxy)nonacosanoic acid, 29-mercapto-29-thioxononacosanoic acid, 29-(hydroxyamino)-29-oxononacosanoic acid, 29-[(mercaptocarbonothioyl)amino]nonacosanoic acid, [29-(hydroxyamino)-29-oxononacosyl]phosphonic acid, [29-(phosphonooxy)nonacosyl]phosphonic acid, {29-[(mercaptocarbonothioyl)amino]nonacosyl}phosphonic acid, 29-phosphonononacosane(dithioic) acid, [29-(phosphonooxy)nonacosyl]carbamodithioic acid, 29-(hydroxyamino)-29-oxononacosyl dihydrogen phosphate, 29-(phosphonooxy)nonacosane(dithioic) acid, 29-(hydroxyamino)-29-oxononacosane(dithioic) acid, [29-(hydroxyamino)-29-oxononacosyl]carbamodithioic acid, 29-[(mercaptocarbonothioyl)amino]nonacosane(dithioic) acid, 29-aminononacosylmethyldichlorosilane, 29-mercaptononacosylmethyldichlorosilane, 29-isocyanatononacosylmethyldichlorosilane, 29-carboxynonacosylmethyldichlorosilane, 29-hydroxynonacosylmethyldichlorosilane, 29-iodononacosylmethyldichlorosilane, 29-chlorononacosylmethyldichlorosilane, 29-bromononacosylmethyldichlorosilane, 29-aminononacosyltrichlorosilane, 29-mercaptononacosyltrichlorosilane, 29-isocyanatononacosyltrichlorosilane, 29-carboxynonacosyltrichlorosilane, 29-hydroxynonacosyltrichlorosilane, 29-iodononacosyltrichlorosilane, 29-chlorononacosyltrichlorosilane, 29-bromononacosyltrichlorosilane, 29-aminononacosylchlorodimethylsilane, 29-mercaptononacosylchlorodimethylsilane, 29-isocyanatononacosylchlorodimethylsilane, 29-carboxynonacosylchlorodimethylsilane, 29-hydroxynonacosylchlorodimethylsilane, 29-iodononacosylchlorodimethylsilane, 29-chlorononacosylchlorodimethylsilane, 29-bromononacosylchlorodimethylsilane, 29-aminononacosyltriethoxysilane, 29-mercaptononacosyltriethoxysilane, 29-isocyanatononacosyltriethoxysilane, 29-carboxynonacosyltriethoxysilane, 29-hydroxynonacosyltriethoxysilane, 29-iodononacosyltriethoxysilane, 29-chlorononacosyltriethoxysilane, 29-bromononacosyltriethoxysilane, 29-aminononacosyltrimethoxysilane, 29-mercaptononacosyltrimethoxysilane, 29-isocyanatononacosyltrimethoxysilane, 29-carboxynonacosyltrimethoxysilane, 29-hydroxynonacosyltrimethoxysilane, 29-iodononacosyltrimethoxysilane, 29-chlorononacosyltrimethoxysilane, 29-bromononacosyltrimethoxysilane, 29-bromononacosan-1-ol, 29-bromononacosanoic acid, 29-bromononacosyl dihydrogen phosphate, 29-bromononacosan-1-thiol, 29-bromononacosane(dithioc) acid, (29-bromononacosyl)carbamodithioc acid, (29-bromononacosyl)phosphonic acid, (29-bromo)-N-hydroxynonacosanamide, 29-chliorononacosan-1-ol, 29-chlorononacosanoic acid, 29-chlorononacosyl dihydrogen phosphate, 29-chlorononacosan-1-thiol, 29-chlorononacosane(dithioc) acid, (29-chlorononacosyl)carbamodithioc acid, (29-chlorononacosyl)phosphonic acid, (29-chloro)-N-hydroxynonacosanamide, 29-iodononacosan-1-ol, 29-iodononacosanoic acid, 29-iodononacosyl dihydrogen phosphate, 29-iodononacosan-1-thiol, 29-iodononacosane(dithioc) acid, (29-iodononacosyl)carbamodithioc acid, (29-iodononacosyl)phosphonic acid, (29-iodo)-N-hydroxynonacosanamide, 29-isocyanatononacosan-1-ol, 29-isocyanatononacosanoic acid, 29-isocyanatononacosyl dihydrogen phosphate, 29-isocyanatononacosan-1-thiol, 29-isocyanatononacosane(dithioc) acid, (29-isocyanatononacosyl)carbamodithioc acid, (29-isocyanatononacosyl)phosphonic acid, (29-isocyanato)-N-hydroxynonacosanamide, 30-aminotriacontan-1-ol, 30-aminotriacontanoic acid, 30-aminotriacontyl dihydrogen phosphate, 30-aminotriacontan-1-thiol, 30-aminotriacontane(dithioc) acid, (30-aminotriacontyl)carbamodithioc acid, (30-aminotriacontyl)phosphonic acid, (30-amino)-N-hydroxytriacontanamide, N-hydroxy-30-mercaptotriacontanamide, (30-mercaptotriacontyl)phosphonic acid, 30-mercaptotriacontan-1-ol, 30-mercaptotriacontanoic acid, 30-mercaptotriacontyl dihydrogen phosphate, 30-mercaptotriacontane(dithioc) acid, (30-mercaptotriacontyl)carbamodithioc acid, 30-hydroxytriacontanoic acid, 30-hydroxytriacontyl dihydrogen phosphate, 30-hydroxytriacontane(dithioc) acid, (30-hydroxytriacontyl)carbamodithioc acid, N,30-dihydroxytriacontanamide, (30-hydroxytriacontyl)phosphonic acid, 30-phosphonotriacontanoic acid, 30-(phosphonooxy)triacontanoic acid, 30-mercapto-30-thioxotriacontanoic acid, 30-(hydroxyamino)-30-oxotriacontanoic acid, 30-[(mercaptocarbonothioyl)amino]triacontanoic acid, [30-(hydroxyamino)-30-oxotriacontyl]phosphonic acid, [30-(phosphonooxy)triacontyl]phosphonic acid, {30-[(mercaptocarbonothioyl)amino]triacontyl}phosphonic acid, 30-phosphonotriacontane(dithioic) acid, [30-(phosphonooxy)triacontyl]carbamodithioic acid, 30-(hydroxyamino)-30-oxotriacontyl dihydrogen phosphate, 30-(phosphonooxy)triacontane(dithioic) acid, 30-(hydroxyamino)-30-oxotriacontane(dithioic) acid, [30-(hydroxyamino)-30-oxotriacontyl]carbamodithioic acid, 30-[(mercaptocarbonothioyl)amino]triacontane(dithioic) acid, 30-aminotriacontylmethyldichlorosilane, 30-mercaptotriacontylmethyldichlorosilane, 30-isocyanatotriacontylmethyldichlorosilane, 30-carboxytriacontylmethyldichlorosilane, 30-hydroxytriacontylmethyldichlorosilane, 30-iodotriacontylmethyldichlorosilane, 30-chlorotriacontylmethyldichlorosilane, 30-bromotriacontylmethyldichlorosilane, 30-aminotriacontyltrichlorosilane, 30-mercaptotriacontyltrichlorosilane, 30-isocyanatotriacontyltrichlorosilane, 30-carboxytriacontyltrichlorosilane, 30-hydroxytriacontyltrichlorosilane, 30-iodotriacontyltrichlorosilane, 30-chlorotriacontyltrichlorosilane, 30-bromotriacontyltrichlorosilane, 30-aminotriacontylchlorodimethylsilane, 30-mercaptotriacontylchlorodimethylsilane, 30-isocyanatotriacontylchlorodimethylsilane, 30-carboxytriacontylchlorodimethylsilane, 30-hydroxytriacontylchlorodimethylsilane, 30-iodotriacontylchlorodimethylsilane, 30-chlorotriacontylchlorodimethylsilane, 30-bromotriacontylchlorodimethylsilane, 30-aminotriacontyltriethoxysilane, 30-mercaptotriacontyltriethoxysilane, 30-isocyanatotriacontyltriethoxysilane, 30-carboxytriacontyltriethoxysilane, 30-hydroxytriacontyltriethoxysilane, 30-iodotriacontyltriethoxysilane, 30-chlorotriacontyltriethoxysilane, 30-bromotriacontyltriethoxysilane, 30-aminotriacontyltrimethoxysilane, 30-mercaptotriacontyltrimethoxysilane, 30-isocyanatotriacontyltrimethoxysilane, 30-carboxytriacontyltrimethoxysilane, 30-hydroxytriacontyltrimethoxysilane, 30-iodotriacontyltrimethoxysilane, 30-chlorotriacontyltrimethoxysilane, 30-bromotriacontyltrimethoxysilane, 30-bromotriacontan-1-ol, 30-bromotriacontanoic acid, 30-bromotriacontyl dihydrogen phosphate, 30-bromotriacontan-1-thiol, 30-bromotriacontane(dithioc) acid, (30-bromotriacontyl)carbamodithioc acid, (30-bromotriacontyl)phosphonic acid, (30-bromo)-N-hydroxytriacontanamide, 30-chlorotriacontan-1-ol, 30-chlorotriacontanoic acid, 30-chlorotriacontyl dihydrogen phosphate, 30-chlorotriacontan-1-thiol, 30-chlorotriacontane(dithioc) acid, (30-chlorotriacontyl)carbamodithioc acid, (30-chlorotriacontyl)phosphonic acid, (30-chloro)-N-hydroxytriacontanamide, 30-iodotriacontan-1-ol, 30-iodotriacontanoic acid, 30-iodotriacontyl dihydrogen phosphate, 30-iodotriacontan-1-thiol, 30-iodotriacontane(dithioc) acid, (30-iodotriacontyl)carbanodithioc acid, (30-iodotriacontyl)phosphonic acid, (30-iodo)-N-hydroxytriacontanamide, 30-isocyanatotriacontan-1-ol, 30-isocyanatotriacontanoic acid, 30-isocyanatotriacontyl dihydrogen phosphate, 30-isocyanatotriacontan-1-thiol, 30-iso cyanatotriacontane(dithioc) acid, (30-isocyanatotriacontyl)carbamodithioc acid, (30-isocyanatotriacontyl)phosphonic acid, (30-isocyanato)-N-hydroxytriacontanamide, 31-aminohentriacontan-1-ol, 31-aminohentriacontanoic acid, 31-aminohentriacontyl dihydrogen phosphate, 31-aminohentriacontan-1-thiol, 31-aminohentriacontane(dithioc) acid, (31-aminohentriacontyl)carbamodithioc acid, (31-aminohentriacontyl)phosphonic acid, (31-amino)-N-hydroxyhentriacontanamide, N-hydroxy-31-mercaptohentriacontanamide, (31-mercaptohentriacontyl)phosphonic acid, 31-mercaptohentriacontan-1-ol, 31-mercaptohentriacontanoic acid, 31-mercaptohentriacontyl dihydrogen phosphate, 31-mercaptohentriacontane(dithioc) acid, (31-mercaptohentriacontyl)carbamodithioc acid, 31-hydroxyhentriacontanoic acid, 31-hydroxyhentriacontyl dihydrogen phosphate, 31-hydroxyhentriacontane(dithioc) acid, (31-hydroxyhentriacontyl)carbamodithioc acid, N,31-dihydroxyhentriacontanamide, (31-hydroxyhentriacontyl)phosphonic acid, 31-phosphonohentriacontanoic acid, 31-(phosphonooxy)hentriacontanoic acid, 31-mercapto-31-thioxohentriacontanoic acid, 31-(hydroxyamino)-31-oxohentriacontanoic acid, 31-[(mercaptocarbonothioyl)amino]hentriacontanoic acid, [31-(hydroxyamino)-31-oxohentriacontyl]phosphonic acid, [31-(phosphonooxy)hentriacontyl]phosphonic acid, {31-[(mercaptocarbonothioyl)amino]hentriacontyl}phosphonic acid, 31-phosphonohentriacontane(dithioic) acid, [31-(phosphonooxy)hentriacontyl]carbamodithioic acid, 31-(hydroxyamino)-31-oxohentriacontyl dihydrogen phosphate, 31-(phosphonooxy)hentriacontane(dithioic) acid, 31-(hydroxyamino)-31-oxohentriacontane(dithioic) acid, [31-(hydroxyamino)-31-oxohentriacontyl]carbamodithioic acid, 31-[(mercaptocarbonothioyl)amino]hentriacontane(dithioic) acid, 31-aminohentriacontylmethyldichlorosilane, 31-mercaptohentriacontylmethyldichlorosilane, 31-isocyanatohentriacontylmethyldichlorosilane, 31-carboxyhentriacontylmethyldichlorosilane, 31-hydroxyhentriacontylmethyldichlorosilane, 31-iodohentriacontylmethyldichlorosilane, 31-chlorohentriacontylmethyldichlorosilane, 31-bromohentriacontylmethyldichlorosilane, 31-aminohentriacontyltrichlorosilane, 31-mercaptohentriacontyltrichlorosilane, 31-isocyanatohentriacontyltrichlorosilane, 31-carboxyhentriacontyltrichlorosilane, 31-hydroxyhentriacontyltrichlorosilane, 31-iodohentriacontyltrichlorosilane, 31-chlorohentriacontyltrichlorosilane, 31-bromohentriacontyltrichlorosilane, 31-aminohentriacontylchlorodimethylsilane, 31-mercaptohentriacontylchlorodimethylsilane, 31-isocyanatohentriacontylchlorodimethylsilane, 31-carboxyhentriacontylchlorodimethylsilane, 31-hydroxyhentriacontylchlorodimethylsilane, 31-iodohentriacontylchlorodimethylsilane, 31-chlorohentriacontylchlorodimethylsilane, 31-bromohentriacontylchlorodimethylsilane, 31-aminohentriacontyltriethoxysilane, 31-mercaptohentriacontyltriethoxysilane, 31-isocyanatohentriacontyltriethoxysilane, 31-carboxyhentriacontyltriethoxysilane, 31-hydroxyhentriacontyltriethoxysilalie, 31-iodohentriacontyltriethoxysilane, 31-chlorohentriacontyltriethoxysilane, 31-bromohentriacontyltriethoxysilane, 31-aminohentriacontyltrimethoxysilane, 31-mercaptohentriacontyltrimethoxysilane, 31-isocyanatohentriacontyltrimethoxysilane, 31-carboxyhentriacontyltrimethoxysilane, 31-hydroxyhentriacontyltrimethoxysilane, 31-iodohentriacontyltrimethoxysilane, 31-chlorohentriacontyltrimethoxysilane, 31-bromohentriacontyltrimethoxysilane, 31-bromohentriacontan-1-ol, 31-bromohentriacontanoic acid, 31-bromohentriacontyl dihydrogen phosphate, 31-bromohentriacontan-1-thiol, 31-bromohentriacontane(dithioc) acid, (31-bromohentriacontyl)carbamodithioc acid, (31-bromohentriacontyl)phosphonic acid, (31-bromo)-N-hydroxyhentriacontanamide, 31-chlorohentriacontan-1-ol, 31-chlorohentriacontanoic acid, 31-chlorohentriacontyl dihydrogen phosphate, 31-chlorohentriacontan-1-thiol, 31-chlorohentriacontane(dithioc) acid, (31-chlorohentriacontyl)carbamodithioc acid, (31-chlorohentriacontyl)phosphonic acid, (31-chloro)-N-hydroxyhentriacontanamide, 31-iodohentriacontan-1-ol, 31-iodohentriacontanoic acid, 31-iodohentriacontyl dihydrogen phosphate, 31-iodohentriacontan-1-thiol, 31-iodohentriacontane(dithioc) acid, (31-iodohentriacontyl)carbamodithioc acid, (31-iodohentriacontyl)phosphonic acid, (31-iodo)-N-hydroxyhentriacontanamide, 31-isocyanatohentriacontan-1-ol, 31-isocyanatohentriacontanoic acid, 31-isocyanatohentriacontyl dihydrogen phosphate, 31-isocyanatohentriacontan-1-thiol, 31-isocyanatohentriacontane(dithioc) acid, (31-isocyanatohentriacontyl)carbamodithioc acid, (31-isocyanatohentriacontyl)phosphonic acid, (31-isocyanato)-N-hydroxyhentriacontanamide, 32-aminodotriacontan-1-ol, 32-aminodotriacontanoic acid, 32-aminodotriacontyl dihydrogen phosphate, 32-aminodotriacontan-1-thiol, 32-aminodotriacontane(dithioc) acid, (32-aminodotriacontyl)carbamodithioc acid, (32-aminodotriacontyl)phosphonic acid, (32-amino)-N-hydroxydotriacontanamide, N-hydroxy-32-mercaptodotriacontanamide, (32-mercaptodotriacontyl)phosphonic acid, 32-mercaptodotriacontan-1-ol, 32-mercaptodotriacontanoic acid, 32-mercaptodotriacontyl dihydrogen phosphate, 32-mercaptodotriacontane(dithioc) acid, (32-mercaptodotriacontyl)carbamodithioc acid, 32-hydroxydotriacontanoic acid, 32-hydroxydotriacontyl dihydrogen phosphate, 32-hydroxydotriacontane(dithioc) acid, (32-hydroxydotriacontyl)carbamodithioc acid, N,32-dihydroxydotriacontanamide, (32-hydroxydotriacontyl)phosphonic acid, 32-phosphonodotriacontanoic acid, 32-(phosphonooxy)dotriacontanoic acid, 32-mercapto-32-thioxodotriacontanoic acid, 32-(hydroxyamino)-32-oxodotriacontanoic acid, 32-[(mercaptocarbonothioyl)amino]dotriacontanoic acid, [32-(hydroxyamino)-32-oxodotriacontyl]phosphonic acid, [32-(phosphonooxy)dotriacontyl]phosphonic acid, {32-[(mercaptocarbonothioyl)amino]dotriacontyl}phosphonic acid, 32-phosphonodotriacontane(dithioic) acid, [32-(phosphonooxy)dotriacontyl]carbamodithioic acid, 32-(hydroxyamino)-32-oxodotriacontyl dihydrogen phosphate, 32-(phosphonooxy)dotriacontane(dithioic) acid, 32-(hydroxyamino)-32-oxodotriacontane(dithioic) acid, [32-(hydroxyamino)-32-oxodotriacontyl]carbamodithioic acid, 32-[(mercaptocarbonothioyl)amino]dotriacontane(dithioic) acid, 32-aminodotriacontylmethyldichlorosilane, 32-mercaptodotriacontylmethyldichlorosilane, 32-isocyanatodotriacontylmethyldichlorosilane, 32-carboxydotriacontylmethyldichlorosilane, 32-hydroxydotriacontylmethyldichlorosilane, 32-iododotriacontylmethyldichlorosilane, 32-chlorodotriacontylmethyldichlorosilane, 32-bromodotriacontylmethyldichlorosilane, 32-aminodotriacontyltrichlorosilane, 32-mercaptodotriacontyltrichlorosilane, 32-isocyanatodotriacontyltrichlorosilane, 32-carboxydotriacontyltrichlorosilane, 32-hydroxydotriacontyltrichlorosilane, 32-iododotriacontyltrichlorosilane, 32-chlorodotriacontyltrichlorosilane, 32-bromodotriacontyltrichlorosilane, 32-aminodotriacontylchlorodimethylsilane, 32-mercaptodotriacontylchlorodimethylsilane, 32-isocyanatodotriacontylchlorodimethylsilane, 32-carboxydotriacontylchlorodimethylsilane, 32-hydroxydotriacontylchlorodimethylsilane, 32-iododotriacontylchlorodimethylsilane, 32-chlorodotriacontylchlorodimethylsilane, 32-bromodotriacontylchlorodimethylsilane, 32-aminodotriacontyltriethoxysilane, 32-mercaptodotriacontyltriethoxysilane, 32-isocyanatodotriacontyltriethoxysilane, 32-carboxydotriacontyltriethoxysilane, 32-hydroxydotriacontyltriethoxysilane, 32-iododotriacontyltriethoxysilane, 32-chlorodotriacontyltriethoxysilane, 32-bromodotriacontyltriethoxysilane, 32-aminodotriacontyltrimethoxysilane, 32-mercaptodotriacontyltrimethoxysilane, 32-isocyanatodotriacontyltrimethoxysilane, 32-carboxydotriacontyltrimethoxysilane, 32-hydroxydotriacontyltrimethoxysilane, 32-iododotriacontyltrimethoxysilane, 32-chlorodotriacontyltrimethoxysilane, 32-bromodotriacontyltrimethoxysilane, 32-bromodotriacontan-1-ol, 32-bromodotriacontanoic acid, 32-bromodotriacontyl dihydrogen phosphate, 32-bromodotriacontan-1-thiol, 32-bromodotriacontane(dithioc) acid, (32-bromodotriacontyl)carbamodithioc acid, (32-bromodotriacontyl)phosphonic acid, (32-bromo)-N-hydroxydotriacontanamide, 32-chlorodotriacontan-1-ol, 32-chlorodotriacontanoic acid, 32-chlorodotriacontyl dihydrogen phosphate, 32-chlorodotriacontan-1-thiol, 32-chlorodotriacontane(dithioc) acid, (32-chlorodotriacontyl)carbamodithioc acid, (32-chlorodotriacontyl)phosphonic acid, (32-chloro)-N-hydroxydotriacontanamide, 32-iododotriacontan-1-ol, 32-iododotriacontanoic acid, 32-iododotriacontyl dihydrogen phosphate, 32-iododotriacontan-1-thiol, 32-iododotriacontane(dithioc) acid, (32-iododotriacontyl)carbamodithioc acid, (32-iododotriacontyl)phosphonic acid, (32-iodo)-N-hydroxydotriacontanamide, 32-isocyanatodotriacontan-1-ol, 32-isocyanatodotriacontanoic acid, 32-isocyanatodotriacontyl dihydrogen phosphate, 32-isocyanatodotriacontan-1-thiol, 32-isocyanatodotriacontane(dithioc) acid, (32-isocyanatodotriacontyl)carbamodithioc acid, (32-isocyanatodotriacontyl)phosphonic acid, (32-isocyanato)-N-hydroxydotriacontanamide, 33-aminotritriacontan-1-ol, 33-aminotritriacontanoic acid, 33-aminotritriacontyl dihydrogen phosphate, 33-aminotritriacontan-1-thiol, 33-aminotritriacontane(dithioc) acid, (33-aminotritriacontyl)carbamodithioc acid, (33-aminotritriacontyl)phosphonic acid, (33-amino)-N-hydroxytritriacontanamide, N-hydroxy-33-mercaptotritriacontanamide, (33-mercaptotritriacontyl)phosphonic acid, 33-mercaptotritriacontan-1-ol, 33-mercaptotritriacontanoic acid, 33-mercaptotritriacontyl dihydrogen phosphate, 33-mercaptotritriacontane(dithioc) acid, (33-mercaptotritriacontyl)carbamodithioc acid, 33-hydroxytritriacontanoic acid, 33-hydroxytritriacontyl dihydrogen phosphate, 33-hydroxytritriacontane(dithioc) acid, (33-hydroxytritriacontyl)carbamodithioc acid, N,33-dihydroxytritriacontanamide, (33-hydroxytritriacontyl)phosphonic acid, 33-phosphonotritriacontanoic acid, 33-(phosphonooxy)tritriacontanoic acid, 33-mercapto-33-thioxotritriacontanoic acid, 33-(hydroxyamino)-33-oxotritriacontanoic acid, 33-[(mercaptocarbonothioyl)amino]tritriacontanoic acid, [33-(hydroxyamino)-33-oxotritriacontyl]phosphonic acid, [33-(phosphonooxy)tritriacontyl]phosphonic acid, {33-[(mercaptocarbonothioyl)amino]tritriacontyl}phosphonic acid, 33-phosphonotritriacontane(dithioic) acid, [33-(phosphonooxy)tritriacontyl]carbamodithioic acid, 33-(hydroxyamino)-33-oxotritriacontyl dihydrogen phosphate, 33-(phosphonooxy)tritriacontane(dithioic) acid, 33-(hydroxyamino)-33-oxotritriacontane(dithioic) acid, [33-(hydroxyamino)-33-oxotritriacontyl]carbamodithioic acid, 33-[(mercaptocarbonothioyl)amino]tritriacontane(dithioic) acid, 33-aminotritriacontylmethyldichlorosilane, 33-mercaptotritriacontylmethyldichlorosilane, 33-isocyanatotritriacontylmethyldichlorosilane, 33-carboxytritriacontylmethyldichlorosilane, 33-hydroxytritriacontylmethyldichlorosilane, 33-iodotritriacontylmethyldichlorosilane, 33-chlorotritriacontylmethyldichlorosilane, 33-bromotritriacontylmethyldichlorosilane, 33-aminotritriacontyltrichlorosilane, 33-mercaptotritriacontyltrichlorosilane, 33-isocyanatotritriacontyltrichlorosilane, 33-carboxytritriacontyltrichlorosilane, 33-hydroxytritriacontyltrichlorosilane, 33-iodotritriacontyltrichlorosilane, 33-chlorotritriacontyltrichlorosilane, 33-bromotritriacontyltrichlorosilane, 33-aminotritriacontylchlorodimethylsilane, 33-mercaptotritriacontylchlorodimethylsilane, 33-isocyanatotritriacontylchlorodimethylsilane, 33-carboxytritriacontylchlorodimethylsilane, 33-hydroxytritriacontylchlorodimethylsilane, 33-iodotritriacontylchlorodimethylsilane, 33-chlorotritriacontylchlorodimethylsilane, 33-bromotritriacontylchlorodimethylsilane, 33-aminotritriacontyltriethoxysilane, 33-mercaptotritriacontyltriethoxysilane, 33-isocyanatotritriacontyltriethoxysilane, 33-carboxytritriacontyltriethoxysilane, 33-hydroxytritriacontyltriethoxysilane, 33-iodotritriacontyltriethoxysilane, 33-chlorotritriacontyltriethoxysilane, 33-bromotritriacontyltriethoxysilane, 33-aminotritriacontyltrimethoxysilane, 33-mercaptotritriacontyltrimethoxysilane, 33-isocyanatotritriacontyltrimethoxysilane, 33-carboxytritriacontyltrimethoxysilane, 33-hydroxytritriacontyltrimethoxysilane, 33-iodotritriacontyltrimethoxysilane, 33-chlorotritriacontyltrimethoxysilane, 33-bromotritriacontyltlimethoxysilane, 33-bromotritriacontan-1-ol, 33-bromotritriacontanoic acid, 33-bromotritriacontyl dihydrogen phosphate, 33-bromotritriacontan-1-thiol, 33-bromotritriacontane(dithioc) acid, (33-bromotritriacontyl)carbamodithioc acid, (33-bromotritriacontyl)phosphonic acid, (33-bromo)-N-hydroxytritriacontanamide, 33-chlorotritriacontan-1-ol, 33-chlorotritriacontanoic acid, 33-chlorotritriacontyl dihydrogen phosphate, 33-chlorotritriacontan-1-thiol, 33-chlorotritriacontane(dithioc) acid, (33-chlorotritriacontyl)carbamodithioc acid, (33-chlorotritriacontyl)phosphonic acid, (33-chloro)-N-hydroxytritriacontanamide, 33-iodotritriacontan-1-ol, 33-iodotritriacontanoic acid, 33-iodotritriacontyl dihydrogen phosphate, 33-iodotritriacontan-1-thiol, 33-iodotritriacontane(dithioc) acid, (33-iodotritriacontyl)carbamodithioc acid, (33-iodotritriacontyl)phosphonic acid, (33-iodo)-N-hydroxytritriacontanamide, 33-isocyanatotritriacontan-1-ol, 33-isocyanatotritriacontanoic acid, 33-isocyanatotritriacontyl dihydrogen phosphate, 33-isocyanatotritriacontan-1-thiol, 33-isocyanatotritriacontane(dithioc) acid, (33-isocyanatotritriacontyl)carbamodithioc acid, (33-isocyanatotritriacontyl)phosphonic acid, (33-isocyanato)-N-hydroxytritriacontanamide, 34-aminotetratriacontan-1-ol, 34-aminotetratriacontanoic acid, 34-aminotetratriacontyl dihydrogen phosphate, 34-aminotetratriacontan-1-thiol, 34-aminotetratriacontane(dithioc) acid, (34-aminotetratriacontyl)carbamodithioc acid, (34-aminotetratriacontyl)phosphonic acid, (34-amino)-N-hydroxytetratriacontanamide, N-hydroxy-34-mercaptotetratriacontanamide, (34-mercaptotetratriacontyl)phosphonic acid, 34-mercaptotetratriacontan-1-ol, 34-mercaptotetratriacontanoic acid, 34-mercaptotetratriacontyl dihydrogen phosphate, 34-mercaptotetratriacontane(dithioc) acid, (34-mercaptotetratriacontyl)carbamodithioc acid, 34-hydroxytetratriacontanoic acid, 34-hydroxytetratriacontyl dihydrogen phosphate, 34-hydroxytetratriacontane(dithioc) acid, (34-hydroxytetratriacontyl)carbamodithioc acid, N,34-dihydroxytetratriacontanamide, (34-hydroxytetratriacontyl)phosphonic acid, 34-phosphonotetratriacontanoic acid, 34-(phosphonooxy)tetratriacontanoic acid, 34-mercapto-34-thioxotetratriacontanoic acid, 34-(hydroxyamino)-34-oxotetratriacontanoic acid, 34-[(mercaptocarbonothioyl)amino]tetratriacontanoic acid, [34-(hydroxyamino)-34-oxotetratriacontyl]phosphonic acid, [34-(phosphonooxy)tetratriacontyl]phosphonic acid, {34-[(mercaptocarbonothioyl)amino]tetratriacontyl}phosphonic acid, 34-phosphonotetratriacontane(dithioic) acid, [34-(phosphonooxy)tetratriacontyl]carbamodithioic acid, 34-(hydroxyamino)-34-oxotetratriacontyl dihydrogen phosphate, 34-(phosphonooxy)tetratriacontane(dithioic) acid, 34-(hydroxyamino)-34-oxotetratriacontane(dithioic) acid, [34-(hydroxyamino)-34-oxotetratriacontyl]carbamodithioic acid, 34-[(mercaptocarbonothioyl)amino]tetratriacontane(dithioic) acid, 34-aminotetratriacontylmethyldichlorosilane, 34-mercaptotetratriacontylmethyldichlorosilane, 34-isocyanatotetratriacontylmethyldichlorosilane, 34-carboxytetratriacontylmethyldichlorosilane, 34-hydroxytetratriacontylmethyldichlorosilane, 34-iodotetratriacontylmethyldichlorosilane, 34-chlorotetratriacontylmethyldichlorosilane, 34-bromotetratriacontylmethyldichlorosilane, 34-aminotetratriacontyltrichlorosilane, 34-mercaptotetratriacontyltrichlorosilane, 34-isocyanatotetratriacontyltrichlorosilane, 34-carboxytetratriacontyltrichlorosilane, 34-hydroxytetratriacontyltrichlorosilane, 34-iodotetratriacontyltrichlorosilane, 34-chlorotetratriacontyltrichlorosilane, 34-bromotetratriacontyltrichlorosilane, 34-aminotetratriacontylchlorodimethylsilane, 34-mercaptotetratriacontylchlorodimethylsilane, 34-isocyanatotetratriacontylchlorodimethylsilane, 34-carboxytetratriacontylchlorodimethylsilane, 34-hydroxytetratriacontylchlorodimethylsilane, 34-iodotetratriacontylchlorodimethylsilane, 34-chlorotetratriacontylchlorodimethylsilane, 34-bromotetratriacontylchlorodimethylsilane, 34-aminotetratriacontyltriethoxysilane, 34-mercaptotetratriacontyltriethoxysilane, 34-isocyanatotetratriacontyltriethoxysilane, 34-carboxytetratriacontyltriethoxysilane, 34-hydroxytetratriacontyltriethoxysilane, 34-iodotetratriacontyltriethoxysilane, 34-chlorotetratriacontyltriethoxysilane, 34-bromotetratriacontyltriethoxysilane, 34-aminotetratriacontyltrimethoxysilane, 34-mercaptotetratriacontyltrimethoxysilane, 34-isocyanatotetratriacontyltrimethoxysilane, 34-carboxytetratriacontyltrimethoxysilane, 34-hydroxytetratriacontyltrimethoxysilane, 34-iodotetratriacontyltrimethoxysilane, 34-chlorotetratriacontyltrimethoxysilane, 34-bromotetratriacontyltrimethoxysilane, 34-bromotetratriacontan-1-ol, 34-bromotetratriacontanoic acid, 34-bromotetratriacontyl dihydrogen phosphate, 34-bromotetratriacontan-1-thiol, 34-bromotetratriacontane(dithioc) acid, (34-bromotetratriacontyl)carbamodithioc acid, (34-bromotetratriacontyl)phosphonic acid, (34-bromo)-N-hydroxytetratriacontanamide, 34-chlorotetratriacontan-1-ol, 34-chlorotetratriacontanoic acid, 34-chlorotetratriacontyl dihydrogen phosphate, 34-chlorotetratriacontan-1-thiol, 34-chlorotetratriacontane(dithioc) acid, (34-chlorotetratriacontyl)carbamodithioc acid, (34-chlorotetratriacontyl)phosphonic acid, (34-chloro)-N-hydroxytetratriacontanamide, 34-iodotetratriacontan-1-ol, 34-iodotetratriacontanoic acid, 34-iodotetratriacontyl dihydrogen phosphate, 34-iodotetratriacontan-1-thiol, 34-iodotetratriacontane(dithioc) acid, (34-iodotetratriacontyl)carbamodithioc acid, (34-iodotetratriacontyl)phosphonic acid, (34-iodo)-N-hydroxytetratriacontanamide, 34-isocyanatotetratriacontan-1-ol, 34-isocyanatotetratriacontanoic acid, 34-isocyanatotetratriacontyl dihydrogen phosphate, 34-isocyanatotetratriacontan-1-thiol, 34-isocyanatotetratriacontane(dithioc) acid, (34-isocyanatotetratriacontyl)carbamodithioc acid, (34-isocyanatotetratriacontyl)phosphonic acid, (34-isocyanato)-N-hydroxytetratriacontanamide, 35-aminopentatriacontan-1-ol, 35-aminopentatriacontanoic acid, 35-aminopentatriacontyl dihydrogen phosphate, 35-aminopentatriacontan-1-thiol, 35-aminopentatriacontane(dithioc) acid, (35-aminopentatriacontyl)carbamodithioc acid, (35-aminopentatriacontyl)phosphonic acid, (35-amino)-N-hydroxypentatriacontanamide, N-hydroxy-35-mercaptopentatriacontanamide, (35-mercaptopentatriacontyl)phosphonic acid, 35-mercaptopentatriacontan-1-ol, 35-mercaptopentatriacontanoic acid, 35-mercaptopentatriacontyl dihydrogen phosphate, 35-mercaptopentatriacontane(dithioc) acid, (35-mercaptopentatriacontyl)carbamodithioc acid, 35-hydroxypentatriacontanoic acid, 35-hydroxypentatriacontyl dihydrogen phosphate, 35-hydroxypentatriacontane(dithioc) acid, (35-hydroxypentatriacontyl)carbamodithioc acid, N,35-dihydroxypentatriacontanamide, (35-hydroxypentatriacontyl)phosphonic acid, 35-phosphonopentatriacontanoic acid, 35-(phosphonooxy)pentatriacontanoic acid, 35-mercapto-35-thioxopentatriacontanoic acid, 35-(hydroxyamino)-35-oxopentatriacontanoic acid, 35-[(mercaptocarbonothioyl)amino]pentatriacontanoic acid, [35-(hydroxyamino)-35-oxopentatriacontyl]phosphonic acid, [35-(phosphonooxy)pentatriacontyl]phosphonic acid, {35-[(mercaptocarbonothioyl)amino]pentatriacontyl}phosphonic acid, 35-phosphonopentatriacontane(dithioic) acid, [35-(phosphonooxy)pentatriacontyl]carbamodithioic acid, 35-(hydroxyamino)-35-oxopentatriacontyl dihydrogen phosphate, 35-(phosphonooxy)pentatriacontane(dithioic) acid, 35-(hydroxyamino)-35-oxopentatriacontane(dithioic) acid, [35-(hydroxyamino)-35-oxopentatriacontyl]carbamodithioic acid, 35-[(mercaptocarbonothioyl)amino]pentatriacontane(dithioic) acid, 35-aminopentatriacontylmethyldichlorosilane, 35-mercaptopentatriacontylmethyldichlorosilane, 35-isocyanatopentatriacontylmethyldichlorosilane, 35-carboxypentatriacontylmethyldichlorosilane, 35-hydroxypentatriacontylmethyldichlorosilane, 35-iodopentatriacontylmethyldichlorosilane, 35-chloropentatriacontylmethyldichlorosilane, 35-bromopentatriacontylmethyldichlorosilane, 35-aminopentatriacontyltrichlorosilane, 35-mercaptopentatriacontyltrichlorosilane, 35-isocyanatopentatriacontyltrichlorosilane, 35-carboxypentatriacontyltrichlorosilane, 35-hydroxypentatriacontyltrichlorosilane, 35-iodopentatriacontyltrichlorosilane, 35-chloropentatriacontyltrichlorosilane, 35-bromopentatriacontyltrichlorosilane, 35-aminopentatriacontylchlorodimethylsilane, 35-mercaptopentatriacontylchlorodimethylsilane, 35-isocyanatopentatriacontylchlorodimethylsilane, 35-carboxypentatriacontylchlorodimethylsilane, 35-hydroxypentatriacontylchlorodimethylsilane, 35-iodopentatriacontylchlorodimethylsilane, 35-chloropentatriacontylchlorodimethylsilane, 35-bromopentatriacontylchlorodimethylsilane, 35-aminopentatriacontyltriethoxysilane, 35-mercaptopentatriacontyltriethoxysilane, 35-isocyanatopentatriacontyltriethoxysilane, 35-carboxypentatriacontyltriethoxysilane, 35-hydroxypentatriacontyltriethoxysilane, 35-iodopentatriacontyltriethoxysilane, 35-chloropentatriacontyltriethoxysilane, 35-bromopentatriacontyltriethoxysilane, 35-aminopentatriacontyltrimethoxysilane, 35-mercaptopentatriacontyltrimethoxysilane, 35-isocyanatopentatriacontyltrimethoxysilane, 35-carboxypentatriacontyltrimethoxysilane, 35-hydroxypentatriacontyltrimethoxysilane, 35-iodopentatriacontyltrimethoxysilane, 35-chloropentatriacontyltrimethoxysilane, 35-bromopentatriacontyltrimethoxysilane, 35-bromopentatriacontan 1-ol, 35-bromopentatriacontanoic acid, 35-bromopentatriacontyl dihydrogen phosphate, 35-bromopentatriacontan-1-thiol, 35-bromopentatriacontane(dithioc) acid, (35-bromopentatriacontyl)carbamodithioc acid, (35-bromopentatriacontyl)phosphonic acid, (35-bromo)-N-hydroxypentatriacontanamide, 35-chloropentatriacontan 1-ol, 35-chloropentatriacontanoic acid, 35-chloropentatriacontyl dihydrogen phosphate, 35-chlioropentatriacontan-1-thiol, 35-chloropentatriacontane(dithioc) acid, (35-chloropentatriacontyl)carbamodithioc acid, (35-chloropentatriacontyl)phosphonic acid, (35-chloro)-N-hydroxypentatriacontanamide, 35-iodopentatriacontan-1-ol, 35-iodopentatriacontanoic acid, 35-iodopentatriacontyl dihydrogen phosphate, 35-iodopentatriacontan-1-thiol, 35-iodopentatriacontane(dithioc) acid, (35-iodopentatriacontyl)carbamodithioc acid, (35-iodopentatriacontyl)phosphonic acid, (35-iodo)-N-hydroxypentatriacontanamide, 35-isocyanatopentatriacontan-1-ol, 35-isocyanatopentatriacontanoic acid, 35-isocyanatopentatriacontyl dihydrogen phosphate, 35-isocyanatopentatriacontan-1-thiol, 35-isocyanatopentatriacontane(dithioc) acid, (35-isocyanatopentatriacontyl)carbamodithioc acid, (35-isocyanatopentatriacontyl)phosphonic acid, (35-isocyanato)-N-hydroxypentatriacontanamide, 36-aminohexatriacontan-1-ol, 36-aminohexatriacontanoic acid, 36-aminohexatriacontyl dihydrogen phosphate, 36-aminohexatriacontan-1-thiol, 36-aminohexatriacontane(dithioc) acid, (36-aminohexatriacontyl)carbamodithioc acid, (36-aminohexatriacontyl)phosphonic acid, (36-amino)-N-hydroxyhexatriacontanamide, N-hydroxy-36-mercaptohexatriacontanamide, (36-mercaptohexatriacontyl)phosphonic acid, 36-mercaptohexatriacontan-1-ol, 36-mercaptohexatriacontanoic acid, 36-mercaptohexatriacontyl dihydrogen phosphate, 36-mercaptohexatriacontane(dithioc) acid, (36-mercaptohexatriacontyl)carbamodithioc acid, 36-hydroxyhexatriacontanoic acid, 36-hydroxyhexatriacontyl dihydrogen phosphate, 36-hydroxyhexatriacontane(dithioc) acid, (36-hydroxyhexatriacontyl)carbamodithioc acid, N,36-dihydroxyhexatriacontanamide, (36-hydroxyhexatriacontyl)phosphonic acid, 36-phosphonohexatriacontanoic acid, 36-(phosphonooxy)hexatriacontanoic acid, 36-mercapto-36-thioxohexatriacontanoic acid, 36-(hydroxyamino)-36-oxohexatriacontanoic acid, 36-[(mercaptocarbonothioyl)amino]hexatriacontanoic acid, [36-(hydroxyamino)-36-oxohexatriacontyl]phosphonic acid, [36-(phosphonooxy)hexatriacontyl]phosphonic acid, {36-[(mercaptocarbonothioyl)amino]hexatriacontyl}phosphonic acid, 36-phosphonohexatriacontane(dithioic) acid, [36-(phosphonooxy)hexatriacontyl]carbamodithioic acid, 36-(hydroxyamino)-36-oxohexatriacontyl dihydrogen phosphate, 36-(phosphonooxy)hexatriacontane(dithioic) acid, 36-(hydroxyamino)-36-oxohexatriacontane(dithioic) acid, [36-(hydroxyamino)-36-oxohexatriacontyl]carbamodithioic acid, 36-[(mercaptocarbonothioyl)amino]hexatriacontane(dithioic) acid, 36-aminohexatriacontylmethyldichlorosilane, 36-mercaptohexatriacontylmethyldichlorosilane, 36-isocyanatohexatriacontylmethyldichlorosilane, 36-carboxyhexatriacontylmethyldichlorosilane, 36-hydroxyhexatriacontylmethyldichlorosilane, 36-iodohexatriacontylmethyldichlorosilane, 36-chlorohexatriacontylmethyldichlorosilane, 36-bromohexatriacontylmethyldichlorosilane, 36-aminohexatriacontyltrichlorosilane, 36-mercaptohexatriacontyltrichlorosilane, 36-isocyanatohexatriacontyltrichlorosilane, 36-carboxyhexatriacontyltrichlorosilane, 36-hydroxyhexatriacontyltrichlorosilane, 36-iodohexatriacontyltrichlorosilane, 36-chlorohexatriacontyltrichlorosilane, 36-bromohexatriacontyltrichlorosilane, 36-aminohexatriacontylchlorodimethylsilane, 36-mercaptohexatriacontylchlorodimethylsilane, 36-isocyanatohexatriacontylchlorodimethylsilane, 36-carboxyhexatriacontylchlorodimethylsilane, 36-hydroxyhexatriacontylchlorodimethylsilane, 36-iodohexatriacontylchlorodimethylsilane, 36-chlorohexatriacontylchlorodimethylsilane, 36-bromohexatriacontylchlorodimethylsilane, 36-aminohexatriacontyltriethoxysilane, 36-mercaptohexatriacontyltriethoxysilane, 36-isocyanatohexatriacontyltriethoxysilane, 36-carboxyhexatriacontyltriethoxysilane, 36-hydroxyhexatriacontyltriethoxysilane, 36-iodohexatriacontyltriethoxysilane, 36-chlorohexatriacontyltriethoxysilane, 36-bromohexatriacontyltriethoxysilane, 36-aminohexatriacontyltrimethoxysilane, 36-mercaptohexatriacontyltrimethoxysilane, 36-isocyanatohexatriacontyltrimethoxysilane, 36-carboxyhexatriacontyltrimethoxysilane, 36-hydroxyhexatriacontyltrimethoxysilane, 36-iodohexatriacontyltrimethoxysilane, 36-chlorohexatriacontyltrimethoxysilane, 36-bromohexatriacontyltrimethoxysilane, 36-bromohexatriacontan-1-ol, 36-bromohexatriacontanoic acid, 36-bromohexatriacontyl dihydrogen phosphate, 36-bromohexatriacontan-1-thiol, 36-bromohexatriacontane(dithioc) acid, (36-bromohexatriacontyl)carbamodithioc acid, (36-bromohexatriacontyl)phosphonic acid, (36-bromo)-N-hydroxyhexatriacontanamide, 36-chlorohexatriacontan-1-ol, 36-chlorohexatriacontanoic acid, 36-chlorohexatriacontyl dihydrogen phosphate, 36-chlorohexatriacontan-1-thiol, 36-chlorohexatriacontane(dithioc) acid, (36-chlorohexatriacontyl)carbamodithioc acid, (36-chlorohexatriacontyl)phosphonic acid, (36-chloro)-N-hydroxyhexatriacontanamide, 36-iodohexatriacontan-1-ol, 36-iodohexatriacontanoic acid, 36-iodohexatriacontyl dihydrogen phosphate, 36-iodohexatriacontan-1-thiol, 36-iodohexatriacontane(dithioc) acid, (36-iodohexatriacontyl)carbamodithioc acid, (36-iodohexatriacontyl)phosphonic acid, (36-iodo)-N-hydroxyhexatriacontanamide, 36-isocyanatohexatriacontan-1-ol, 36-isocyanatohexatriacontanoic acid, 36-isocyanatohexatriacontyl dihydrogen phosphate, 36-isocyanatohexatriacontan-1-thiol, 36-isocyanatohexatriacontane(dithioc) acid, (36-isocyanatohexatriacontyl)carbamodithioc acid, (36-isocyanatohexatriacontyl)phosphonic acid, (36-isocyanato)-N-hydroxyhexatriacontanamide, 37-aminoheptatriacontan-1-ol, 37-aminoheptatriacontanoic acid, 37-aminoheptatriacontyl dihydrogen phosphate, 37-aminoheptatriacontan-1-thiol, 37-aminoheptatriacontane(dithioc) acid, (37-aminoheptatriacontyl)carbamodithioc acid, (37-aminoheptatriacontyl)phosphonic acid, (37-amino)-N-hydroxyheptatriacontanamide, N-hydroxy-37-mercaptoheptatriacontanamide, (37-mercaptoheptatriacontyl)phosphonic acid, 37-mercaptoheptatriacontan-1-ol, 37-mercaptoheptatriacontanoic acid, 37-mercaptoheptatriacontyl dihydrogen phosphate, 37-mercaptoheptatriacontane(dithioc) acid, (37-mercaptoheptatriacontyl)carbamodithioc acid, 37-hydroxyheptatriacontanoic acid, 37-hydroxyheptatriacontyl dihydrogen phosphate, 37-hydroxyheptatriacontane(dithioc) acid, (37-hydroxyheptatriacontyl)carbamodithioc acid, N,37-dihydroxyheptatriacontanamide, (37-hydroxyheptatriacontyl)phosphonic acid, 37-phosphonoheptatriacontanoic acid, 37-(phosphonooxy)heptatriacontanoic acid, 37-mercapto-37-thioxoheptatriacontanoic acid, 37-(hydroxyamino)-37-oxoheptatriacontanoic acid, 37-[(mercaptocarbonothioyl)amino]heptatriacontanoic acid, [37-(hydroxyamino)-37-oxoheptatriacontyl]phosphonic acid, [37-(phosphonooxy)heptatriacontyl]phosphonic acid, {37-[(mercaptocarbonothioyl)amino]heptatriacontyl}phosphonic acid, 37-phosphonoheptatriacontane(dithioic) acid, [37-(phosphonooxy)heptatriacontyl]carbamodithioic acid, 37-(hydroxyamino)-37-oxoheptatriacontyl dihydrogen phosphate, 37-(phosphonooxy)heptatriacontane(dithioic) acid, 37-(hydroxyamino)-37-oxoheptatriacontane(dithioic) acid, [37-(hydroxyamino)-37-oxoheptatriacontyl]carbamodithioic acid, 37-[(mercaptocarbonothioyl)amino]heptatriacontane(dithioic) acid, 37-aminoheptatriacontylmethyldichlorosilane, 37-mercaptoheptatriacontylmethyldichlorosilane, 37-isocyanatoheptatriacontylmethyldichlorosilane, 37-carboxyheptatriacontylmethyldichlorosilane, 37-hydroxyheptatriacontylmethyldichlorosilane, 37-iodoheptatriacontylmethyldichlorosilane, 37-chloroheptatriacontylmethyldichlorosilane, 37-bromoheptatriacontylmethyldichlorosilane, 37-aminoheptatriacontyltrichlorosilane, 37-mercaptoheptatriacontyltrichlorosilane, 37-isocyanatoheptatriacontyltrichlorosilane, 37-carboxyheptatriacontyltrichlorosilane, 37-hydroxyheptatriacontyltrichlorosilane, 37-iodoheptatriacontyltrichlorosilane, 37-chloroheptatriacontyltrichlorosilane, 37-bromoheptatriacontyltrichlorosilane, 37-aminoheptatriacontylchlorodimethylsilane, 37-mercaptoheptatriacontylchlorodimethylsilane, 37-isocyanatoheptatriacontylchlorodimethylsilane, 37-carboxyheptatriacontylchlorodimethylsilane, 37-hydroxyheptatriacontylchlorodimethylsilane, 37-iodoheptatriacontylchlorodimethylsilane, 37-chloroheptatriacontylchlorodimethylsilane, 37-bromoheptatriacontylchlorodimethylsilane, 37-aminoheptatriacontyltriethoxysilane, 37-mercaptoheptatriacontyltriethoxysilane, 37-isocyanatoheptatriacontyltriethoxysilane, 37-carboxyheptatriacontyltriethoxysilane, 37-hydroxyheptatriacontyltriethoxysilane, 37-iodoheptatriacontyltriethoxysilane, 37-chloroheptatriacontyltriethoxysilane, 37-bromoheptatriacontyltriethoxysilane, 37-aminoheptatriacontyltrimethoxysilane, 37-mercaptoheptatriacontyltrimethoxysilane, 37-isocyanatoheptatriacontyltrimethoxysilane, 37-carboxyheptatriacontyltrimethoxysilane, 37-hydroxyheptatriacontyltrimethoxysilane, 37-iodoheptatriacontyltrimethoxysilane, 37-chloroheptatriacontyltrimethoxysilane, 37-bromoheptatriacontyltrimethoxysilane, 37-bromoheptatriacontan-1-ol, 37-bromoheptatriacontanoic acid, 37-bromoheptatriacontyl dihydrogen phosphate, 37-bromoheptatriacontan-1-thiol, 37-bromoheptatriacontane(dithioc) acid, (37-bromoheptatriacontyl)carbamodithioc acid, (37-bromoheptatriacontyl)phosphonic acid, (37-bromo)-N-hydroxyheptatriacontanamide, 37-chloroheptatriacontan-1-ol, 37-chloroheptatriacontanoic acid, 37-chloroheptatriacontyl dihydrogen phosphate, 37-chloroheptatriacontan-1-thiol, 37-chloroheptatriacontane(dithioc) acid, (37-chloroheptatriacontyl)carbamodithioc acid, (37-chloroheptatriacontyl)phosphonic acid, (37-chloro)-N-hydroxyheptatriacontanamide, 37-iodoheptatriacontan-1-ol, 37-iodoheptatriacontanoic acid, 37-iodoheptatriacontyl dihydrogen phosphate, 37-iodoheptatriacontan-1-thiol, 37-iodoheptatriacontane(dithioc) acid, (37-iodoheptatriacontyl)carbamodithioc acid, (37-iodoheptatriacontyl)phosphonic acid, (37-iodo)-N-iydroxyheptatriacontanamide, 37-isocyanatoheptatriacontan-1-ol, 37-isocyanatoheptatriacontanoic acid, 37-isocyanatoheptatriacontyl dihydrogen phosphate, 37-isocyanatoheptatriacontan-1-thiol, 37-isocyanatoheptatriacontane(dithioc) acid, (37-isocyanatoheptatriacontyl)carbamodithioc acid, (37-isocyanatoheptatriacontyl)phosphonic acid, (37-isocyanato)-N-hydroxyheptatriacontanamide, 38-aminooctatriacontan-1-ol, 38-aminooctatriacontanoic acid, 38-aminooctatriacontyl dihydrogen phosphate, 38-aminooctatriacontan-1-thiol, 38-aminooctatriacontane(dithioc) acid, (38-aminooctatriacontyl)carbamodithioc acid, (38-aminooctatriacontyl)phosphonic acid, (38-amino)-N-hydroxyoctatriacontanamide, N-hydroxy-38-mercaptooctatriacontanamide, (38-mercaptooctatriacontyl)phosphonic acid, 38-mercaptooctatriacontan-1-ol, 38-mercaptooctatriacontanoic acid, 38-mercaptooctatriacontyl dihydrogen phosphate, 38-mercaptooctatriacontane(dithioc) acid, (38-mercaptooctatriacontyl)carbamodithioc acid, 38-hydroxyoctatriacontanoic acid, 38-hydroxyoctatriacontyl dihydrogen phosphate, 38-hydroxyoctatriacontane(dithioc) acid, (38-hydroxyoctatriacontyl)carbamodithioc acid, N,38-dihydroxyoctatriacontanamide, (38-hydroxyoctatriacontyl)phosphonic acid, 38-phosphonooctatriacontanoic acid, 38-(phosphonooxy)octatriacontanoic acid, 38-mercapto-38-thioxooctatriacontanoic acid, 38-(hydroxyamino)-38-oxooctatriacontanoic acid, 38-[(mercaptocarbonothioyl)amino]octatriacontanoic acid, [38-(hydroxyamino)-38-oxooctatriacontyl]phosphonic acid, [38-(phosphonooxy)octatriacontyl]phosphonic acid, {38-[(mercaptocarbonothioyl)amino]octatriacontyl}phosphonic acid, 38-phosphonooctatriacontane(dithioic) acid, [38-(phosphonooxy)octatriacontyl]carbamodithioic acid, 38-(hydroxyamino)-38-oxooctatriacontyl dihydrogen phosphate, 38-(phosphonooxy)octatriacontane(dithioic) acid, 38-(hydroxyamino)-38-oxooctatriacontane(dithioic) acid, [38-(hydroxyamino)-38-oxooctatriacontyl]carbamodithioic acid, 38-[(mercaptocarbonothioyl)amino]octatriacontane(dithioic) acid, 38-aminooctatriacontylmethyldichlorosilane, 38-mercaptooctatriacontylmethyldichlorosilane, 38-isocyanatooctatriacontylmethyldichlorosilane, 38-carboxyoctatriacontylmethyldichlorosilane, 38-hydroxyoctatriacontylmethyldichlorosilane, 38-iodooctatriacontylmethyldichlorosilane, 38-chlorooctatriacontylmethyldichlorosilane, 38-bromooctatriacontylmethyldichlorosilane, 38-aminooctatriacontyltrichlorosilane, 38-mercaptooctatriacontyltrichlorosilane, 38-isocyanatooctatriacontyltrichlorosilane, 38-carboxyoctatriacontyltrichlorosilane, 38-hydroxyoctatriacontyltrichlorosilane, 38-iodooctatriacontyltrichlorosilane, 38-chlorooctatriacontyltrichlorosilane, 38-bromooctatriacontyltrichlorosilane, 38-aminooctatriacontylchlorodimethylsilane, 38-mercaptooctatriacontylchlorodimethylsilane, 38-isocyanatooctatriacontylchlorodimethylsilane, 38-carboxyoctatriacontylchlorodimethylsilane, 38-hydroxyoctatriacontylchlorodimethylsilane, 38-iodooctatriacontylchlorodimethylsilane, 38-chlorooctatriacontylchlorodimethylsilane, 38-bromooctatriacontylchlorodimethylsilane, 38-aminooctatriacontyltriethoxysilane, 38-mercaptooctatriacontyltriethoxysilane, 38-isocyanatooctatriacontyltriethoxysilane, 38-carboxyoctatriacontyltriethoxysilane, 38-hydroxyoctatriacontyltriethoxysilane, 38-iodooctatriacontyltriethoxysilane, 38-chlorooctatriacontyltriethoxysilane, 38-bromooctatriacontyltriethoxysilane, 38-aminooctatriacontyltrimethoxysilane, 38-mercaptooctatriacontyltrimethoxysilane, 38-isocyanatooctatriacontyltrimethoxysilane, 38-carboxyoctatriacontyltrimethoxysilane, 38-hydroxyoctatriacontyltrimethoxysilane, 38-iodooctatriacontyltrimethoxysilane, 38-chlorooctatriacontyltrimethoxysilane, 38-bromooctatriacontyltrimethoxysilane, 38-bromooctatriacontan-1-ol, 38-bromooctatriacontanoic acid, 38-bromooctatriacontyl dihydrogen phosphate, 38-bromooctatriacontan-1-thiol, 38-bromooctatriacontane(dithioc) acid, (38-bromooctatriacontyl)carbamodithioc acid, (38-bromooctatriacontyl)phosphonic acid, (38-bromo)-N-hydroxyoctatriacontanamide, 38-chlorooctatriacontan-1-ol, 38-chlorooctatriacontanoic acid, 38-chlorooctatriacontyl dihydrogen phosphate, 38-chlorooctatriacontan-1-thiol, 38-chlorooctatriacontane(dithioc) acid, (38-chlorooctatriacontyl)carbamodithioc acid, (38-chlorooctatriacontyl)phosphonic acid, (38-chloro)-N-hydroxyoctatriacontanamide, 38-iodooctatriacontan-1-ol, 38-iodooctatriacontanoic acid, 38-iodooctatriacontyl dihydrogen phosphate, 38-iodooctatriacontan-1-thiol, 38-iodooctatriacontane(dithioc) acid, (38-iodooctatriacontyl)carbanodithioc acid, (38-iodooctatriacontyl)phosphonic acid, (38-iodo)-N-hydroxyoctatriacontanamide, 38-isocyanatooctatriacontan-1-ol, 38-isocyanatooctatriacontanoic acid, 38-isocyanatooctatriacontyl dihydrogen phosphate, 38-isocyanatooctatriacontan-1-thiol, 38-isocyanatooctatriacontane(dithioc) acid, (38-isocyanatooctatriacontyl)carbamodithioc acid, (38-isocyanatooctatriacontyl)phosphonic acid, (38-isocyanato)-N-hydroxyoctatriacontanamide, 39-aminononatriacontan-1-ol, 39-aminononatriacontanoic acid, 39-aminononatriacontyl dihydrogen phosphate, 39-aminononatriacontan-1-thiol, 39-aminononatriacontane(dithioc) acid, (39-aminononatriacontyl)carbamodithioc acid, (39-aminononatriacontyl)phosphonic acid, (39-amino)-N-hydroxynonatriacontanamide, N-hydroxy-39-mercaptononatriacontanamide, (39-mercaptononatriacontyl)phosphonic acid, 39-mercaptononatriacontan-1-ol, 39-mercaptononatriacontanoic acid, 39-mercaptononatriacontyl dihydrogen phosphate, 39-mercaptononatriacontane(dithioc) acid, (39-mercaptononatriacontyl)carbamodithioc acid, 39-hydroxynonatriacontanoic acid, 39-hydroxynonatriacontyl dihydrogen phosphate, 39-hydroxynonatriacontane(dithioc) acid, (39-hydroxynonatriacontyl)carbamodithioc acid, N,39-dihydroxynonatriacontanamide, (39-hydroxynonatriacontyl)phosphonic acid, 39-phosphonononatriacontanoic acid, 39-(phosphonooxy)nonatriacontanoic acid, 39-mercapto-39-thioxononatriacontanoic acid, 39-(hydroxyamino)-39-oxononatriacontanoic acid, 39-[(mercaptocarbonothioyl)amino]nonatriacontanoic acid, [39-(hydroxyamino)-39-oxononatriacontyl]phosphonic acid, [39-(phosphonooxy)nonatriacontyl]phosphonic acid, {39-[(mercaptocarbonothioyl)amino]nonatriacontyl}phosphonic acid, 39-phosphonononatriacontane(dithioic) acid, [39-(phosphonooxy)nonatriacontyl]carbamodithioic acid, 39-(hydroxyamino)-39-oxononatriacontyl dihydrogen phosphate, 39-(phosphonooxy)nonatriacontane(dithioic) acid, 39-(hydroxyamino)-39-oxononatriacontane(dithioic) acid, [39-(hydroxyamino)-39-oxononatriacontyl]carbamodithioic acid, 39-[(mercaptocarbonothioyl)amino]nonatriacontane(dithioic) acid, 39-aminononatriacontylmethyldichlorosilane, 39-mercaptononatriacontylmethyldichlorosilane, 39-isocyanatononatriacontylmethyldichlorosilane, 39-carboxynonatriacontylmethyldichlorosilane, 39-hydroxynonatriacontylmethyldichlorosilane, 39-iodononatriacontylmethyldichlorosilane, 39-chlorononatriacontylmethyldichlorosilane, 39-bromononatriacontylmethyldichlorosilane, 39-aminononatriacontyltrichlorosilane, 39-mercaptononatriacontyltrichlorosilane, 39-isocyanatononatriacontyltrichlorosilane, 39-carboxynonatriacontyltrichlorosilane, 39-hydroxynonatriacontyltrichlorosilane, 39-iodononatriacontyltrichlorosilane, 39-chlorononatriacontyltrichlorosilane, 39-bromononatriacontyltrichlorosilane, 39-aminononatriacontylchlorodimethylsilane, 39-mercaptononatriacontylchlorodimethylsilane, 39-isocyanatononatriacontylchlorodimethylsilane, 39-carboxynonatriacontylchlorodimethylsilane, 39-hydroxynonatriacontylchlorodimethylsilane, 39-iodononatriacontylchlorodimethylsilane, 39-chlorononatriacontylclilorodimethylsilane, 39-bromononatriacontylchlorodimethylsilane, 39-aminononatriacontyltriethoxysilane, 39-mercaptononatriacontyltriethoxysilane, 39-isocyanatononatriacontyltriethoxysilane, 39-carboxynonatriacontyltriethoxysilane, 39-hydroxynonatriacontyltriethoxysilane, 39-iodononatriacontyltriethoxysilane, 39-chlorononatriacontyltriethoxysilane, 39-bromononatriacontyltriethoxysilane, 39-aminononatriacontyltrimethoxysilane, 39-mercaptononatriacontyltrimethoxysilane, 39-isocyanatononatriacontyltrimethoxysilane, 39-carboxynonatriacontyltrimethoxysilane, 39-hydroxynonatriacontyltrimethoxysilane, 39-iodononatriacontyltrimethoxysilane, 39-chlorononatriacontyltrimethoxysilane, 39-bromononatriacontyltrimethoxysilane, 39-bromononatriacontan-1-ol, 39-bromononatriacontanoic acid, 39-bromononatriacontyl dihydrogen phosphate, 39-bromononatriacontan-1-thiol, 39-bromononatriacontane(dithioc) acid, (39-bromononatriacontyl)carbamodithioc acid, (39-bromononatriacontyl)phosphonic acid, (39-bromo)-N-hydroxynonatriacontanamide, 39-chlorononatriacontan-1-ol, 39-chlorononatriacontanoic acid, 39-chlorononatriacontyl dihydrogen phosphate, 39-chlorononatriacontan-1-thiol, 39-chlorononatriacontane(dithioc) acid, (39-chlorononatriacontyl)carbamodithioc acid, (39-chlorononatriacontyl)phosphonic acid, (39-chloro)-N-hydroxynonatriacontanamide, 39-iodononatriacontan-1-ol, 39-iodononatriacontanoic acid, 39-iodononatriacontyl dihydrogen phosphate, 39-iodononatriacontan-1-thiol, 39-iodononatriacontane(dithioc) acid, (39-iodononatriacontyl)carbamodithioc acid, (39-iodononatriacontyl)phosphonic acid, (39-iodo)-N-hydroxynonatriacontanamide, 39-isocyanatononatriacontan-1-ol, 39-isocyanatononatriacontanoic acid, 39-isocyanatononatriacontyl dihydrogen phosphate, 39-isocyanatononatriacontan-1-thiol, 39-isocyanatononatriacontane(dithioc) acid, (39-isocyanatononatriacontyl)carbamodithioc acid, (39-isocyanatononatriacontyl)phosphonic acid, (39-isocyanato)-N-hydroxynonatriacontanamide.

The SAM molecules include one or more additional functional groups which are able to attach to one or more linker molecules. Linkers are discussed in greater detail in the specification below. Specifically, if the Drug-SAM-forming molecule is designed properly, only the binding group will be present at the implant surface, and only the drug will be present at the implant-tissue interface. This level of precision creates opportunities for a highly consistent dose delivery of a drug. Drug-SAM technology would represent a dramatic improvement over polymer coatings because the SAMs form a molecular layer that is integrated and part of the implant surface. Drug-SAMs will not fracture, flake or otherwise deform, providing considerable advantage over polymer coatings. Since SAMs are not polymers, allergic reactions would be eliminated.

3. Attachment of a Self-Assembled Monolayer Molecule to a Surface and Fabrication of Self-Assembled Monolayers

Any method known to those of ordinary skill in the art can be used to synthesize the SAM molecules of the present invention. These methods include methods of chemical synthesis well-known to those of ordinary skill in the art. The SAM molecules can also be acquired from natural sources as well. Additional information pertaining to the synthesis of SAM molecules can be found in Ulman, 1996; Allara et al., 1991; Tao, 1993; Schlotter et al., 1986; Chau and Porter, 1990; Folkers et al., 1995; Lin et al., 2002; Hofer et al., 2001; Zwahlen et al., 2002; Fadeev and McCarthy, 1999; Helmy and Fadeev, 2002; Marcinko and Fadeev, 2004, each of which is herein specifically incorporated by reference in its entirety for this and all other sections of this specification.

The attachment of the SAM molecule to a surface of the medical device is by any method known to those of ordinary skill in the art. In particular, the SAM molecules may be attached to the surface by covalent binding or non-covalent (ionic) binding. Additional information pertaining to the attachment or binding of SAM molecules to a surface can be found in Ulman, 1996; Allara et al., 1991; Tao, 1993; Schlotter et al., 1986; Chau and Porter, 1990; Folkers et al., 1995; Lin et al., 2002; Hofer et al., 2001; Zwahlen et al., 2002; Fadeev and McCarthy, 1999; Helmy and Fadeev, 2002; Marcinko and Fadeev, 2004, each of which is herein specifically incorporated by reference in its entirety for this and all other sections of this specification.

Attachment of SAM molecules to surface titanium oxide has been described. In particular, there are four known functional groups that bind to surface titanium oxide: alkylphosphoric acids, alkylphosphonic acids, hydroxyamic acids (Folkers et al., 1995), and silanes (Fadeev and McCarthy, 1999; Helmy and Fadeev, 2002; Marcinko and Fadeev, 2004). The preparative chemistry for each of these functional groups is established (Folkers et al., 1995; Anderson and Hendifar, 1959; Ryan et al., 1960; Laane, 1967; Pawsey et al., 2002; Okamoto, 1985, each of which is herein specifically incorporated by reference). The limited reports of monolayers on 316L stainless steel is partly due to the difficulty in activating the surface for deposition (Shustak et al., 2004). However there are some reports that demonstrate the formation of alkyl thiols (Ruan et al., 2002), alkyl amines (Ruan et al., 2002), phosphonic acids (van Alsten, 1999), silane (Meth and Sukenik, 2003) and alkanoic acid (Shustak et al, 2004).

Additional exemplary detail regarding SAM molecules and the fabrication of SAMs can be found in U.S. Patent Application Pub. No. 20040037836, U.S. Pat. No. 6,617,527, U.S. Patent Application Pub. No. 20030158460, U.S. Pat. No. 6,821,529, U.S. Pat. No. 6,723,517, U.S. Pat. No. 6,242,264, U.S. Pat. No. 6,146,767, U.S. Pat. No. 6,025,202, U.S. Pat. No. 5,852,127, U.S. Pat. No. 5,721,131, U.S. Pat. No. 5,609,907, U.S. Patent Application Pub. No. 20030157732, U.S. Patent Application Pub. No. 20030059865, U.S. Patent Application Pub. No. 20020197879, U.S. Patent Application Pub. No. 20020164419, U.S. Patent Application Pub. No. 20020119305, U.S. Patent Application Pub. No. 2004/0062592, U.S. Pat. No. 6,756,354, U.S. Pat. No. 6,617,027, and U.S. Pat. No. 6,440,565, each of which is herein specifically incorporated by reference in its entirety for this and all other sections of this specification.

In some embodiments, a polymer or peptide is attached to the SAM molecule. A “polymer” is defined as a molecule comprised of two or more repeating linked units. One of ordinary skill in the art would be familiar with polymers and polymer chemistry. For example, the polymer may be poly(ethylene glycol). A peptide is defined herein to refer to a consecutive amino acid sequence of from two to about 200 amino acid residues in length. The peptide may be any peptide known to those of ordinary skill in the art. For example, the peptide may be a cellular adhesion peptide, defined herein to refer to a peptide that is capable of forming an attachment to a cell. For example, the tripeptide RGD (arginine-glycine-aspartic acid) or other peptides known to promote cellular adhesion may be employed. Peptides known to promote cellular adhesion are discussed in greater detail in Yang et al., 2005, Biltresse et al., 2005, and Picart et al., 2005, each of which is herein specifically incorporated by reference in its entirety.

The attachment may be any type of attachment known to those of ordinary skill in the art. For example, the attachment may be non-covalent (ionic) or covalent. The purpose of these coatings is to promote or inhibit cellular attachment to the device as needed by the end-user application.

### B. LINKERS

In certain embodiments of the present medical devices and methods, a linker is interposed between a SAM molecule and a therapeutic agent, such that the linker is attached to the SAM molecule and the therapeutic agent by different functional groups of the linker.

1. Definition

A “linker” is defined herein to refer to a molecule comprising two or more functional groups, wherein one of the functional groups is capable of forming an attachment to a SAM molecule, and wherein a second functional group is capable of forming an attachment to a therapeutic agent. Therapeutic agents are discussed in greater detail in the specification below. The attachment to the SAM molecule and to the therapeutic agent can be covalent or non-covalent (ionic). The functional groups of the linker may be identical, or the functional groups may differ. Thus, for example, a linker may include a hydroxyl functional group for covalent binding to a SAM molecule, and an amino functional group for non-covalent binding to a therapeutic agent. Aside from the functional groups, the linker can be of any structure.

Exemplary functional groups of linkers include, but are not limited to, the following: a hydroxyl, a carboxyl, an amino, a phosphate, a phosphonate, a sulfate, a sulfite, a sulfenate, a sulfinate, a sulfonate, a sulfoxide, a sulfone, an amide, an ester, an ketone, an aldehyde, a nitrile, an alkene, an alkyne, an ether, a thiol, a hydroxyamic acid, a silane, a silicate, a carbamodithionate, a dithionate, a mercaptan, a disulfide, a peroxide and a nitronate.

2. Exemplary Linkers

While numerous types of linkers are known which can successfully be employed to conjugate moieties, certain linkers will generally be preferred over other linkers, based on differing pharmacologic characteristics and capabilities.

Exemplary preferred linkers include, but are not limited to, polyethylene glycol, a dendrimer, a molecule comprising a tert-butyl protecting group, a molecule comprising an isobutylene oxide connection, an amino benzyl alcohol, a hydroxy benzyl alcohol connection, an aminobenzene dimethanol, an aminobenzene trimethanol, a hydroxybenzene dimethanol, a hydroxybenzene trimethanol, a vinyl sulfoxide, a substituted vinyl sulfoxide, a substituted methoxymethyl connection, a substituted vinyl ether connection, a carbonate connection, an ester connection, an anhydride connection, a substituted carbamic anhydride connection, a carbonic anhydride connection, an substituted urea connection, a substituted urethane connection, a substituted guanidine connection, a ether connection, a mercaptan connection, a sulfoxide connection, a sulfinate connection, a sulfonate connection, a sulfenate connection, a nitronate connection, a sulfite connection, a sulfate connection, a phosphate connection, a phosphonate connection, a phosphine connection, a silane connection, a silicate connection, a disulfide connection, a peroxide connection, an alkane connection, an alkene connection, an alkyne connection, an iodonium connection, an amino connection, a substituted allyl ether connection, a substituted benzyl ether connection and an imine connection. Linkers that contain a disulfide bond that is sterically “hindered” may be included preventing premature release of the therapeutic agent.

In some embodiments, the linker is further defined as a cross-linking reagent. Cross-linking reagents are used to form molecular bridges that tie together functional groups of two different molecules.

TABLE 1
HETERO-BIFUNCTIONAL CROSS-LINKERS

| | | | Spacer Arm | |
|---|---|---|---|---|
| | | | Length\after cross- | |
| linker | Reactive Toward | Advantages and Applications | linking | |
| SMPT | Primary amines | Greater stability | 11.2 | A |
| | Sulfhydryls | | | |
| SPDP | Primary amines | Thiolation | 6.8 | A |
| | Sulfhydryls | Cleavable cross-linking | | |
| LC-SPDP | Primary amines | Extended spacer arm | 15.6 | A |
| | Sulfhydryls | | | |
| Sulfo-LC- | Primary amines | Extended spacer arm | 15.6 | A |
| SPDP | Sulfhydryls | Water-soluble | | |
| SMCC | Primary amines | Stable maleimide reactive group | 11.6 | A |
| | Sulfhydryls | Enzyme-antibody conjugation | | |
| | | Hapten-carrier protein conjugation | | |
| Sulfo- | Primary amines | Stable maleimide reactive group | 11.6 | A |
| SMCC | Sulfhydryls | Water-soluble | | |
| | | Enzyme-antibody conjugation | | |
| MBS | Primary amines | Enzyme-antibody conjugation | 9.9 | A |
| | Sulfhydryls | Hapten-carrier protein conjugation | | |
| Sulfo- | Primary amines | Water-soluble | 9.9 | A |
| MBS | Sulfhydryls | | | |
| SIAB | Primary amines | Enzyme-antibody conjugation | 10.6 | A |
| | Sulfhydryls | | | |
| Sulfo- | Primary amines | Water-soluble | 10.6 | A |
| SIAB | Sulfhydryls | | | |
| SMPB | Primary amines | Extended spacer arm | 14.5 | A |
| | Sulfhydryls | Enzyme-antibody conjugation | | |
| Sulfo- | Primary amines | Extended spacer arm | 14.5 | A |
| SMPB | Sulfhydryls | Water-soluble | | |
| EDC/Sulfo- | Primary amines | Hapten-Carrier conjugation | 0 | |
| NHS | Carboxyl groups | | | |
| ABH | Carbohydrates | Reacts with sugar groups | 11.9 | A |
| | Nonselective | | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a linker having reasonable stability in blood will be employed. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1986). Another embodiment involves the use of flexible linkers.

U.S. Pat. No. 4,680,338, herein specifically incorporated by reference, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250, both of which are herein specifically incorporated by reference, disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456, herein specifically incorporated by reference, provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

3. Dendrimers and Dendritic Structures

In certain embodiments of the present invention, the linker is further defined as a dendrimer, or dendritic structure. A “dendrimer,” or dendritic structure, is defined herein to refer to cascade-branched, highly defined, synthetic macromolecules, which are characterized by a combination of high number of functional groups and a compact molecular structure (Tomalia and Frechet, 2002). The concept of repetitive growth with branching creates a unique spherical monodisperse dendrimer formation, which is defined by a precise generation number (Tomalia et al., 1990). First-generation dendrimer (G1) will have one branching unit, and a second-generation dendrimer (G2) will have additional branching units, and so forth. (Amir et al., 2003).

In the context of the present invention, a dendrimer could be applied as a linker to attach multiple therapeutic agents to a SAM molecule. The dendrimer or dendritic structure may be capable of disassembly, self-immolation, release by dendritic amplification, or cascade release. More particularly, the dendrimer could be structured to release all of the therapeutic agents with a single cleavage event at the dendrimer's core. Alternatively, the dendrimer could be designed with a trigger that can initiate the fragmentation of the dendrimer molecule to its building blocks in a self-immolative manner with consequence release of the therapeutic agents. Information regarding dendrimers and release of agents from dendrimers is discussed in greater detail in Amir et al. 2003, Madec-Lougerstay et al., 1999 and Lougerstay-Madec et al. 1998, each of which is herein specifically incorporated by reference in its entirety. One of ordinary skill in the art would be familiar with dendrimers, and applications of dendrimers that involve the attachment and release of multiple functional groups.

4. Attachment of Linker to Self-Assembling Monolayer Molecule and to Therapeutic Agent

Any mechanism known to those of skill in the art can be used to attach a linker to the SAM molecule and therapeutic agent. As set forth above, the attachment of the linker to the SAM molecule can be by any method of attachment known to those of ordinary skill in the art. Examples include covalent attachment and non-covalent attachment. Specific examples of such binding include avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, antigen-antibody interactions, or combinations thereof. One of ordinary skill in the art would be very familiar with the chemistry associated with binding two functional groups together.

During fabrication, the steps of attachment between the medical device, SAM molecule, linker, and therapeutic agent can be in any order, and can be performed by any method known to those of ordinary skill in the art. For example, the self-assembling monolayer molecule may be first attached to the medical device, secondly attached to the linker, and then the linker attached to the therapeutic agent. In other embodiments, the linker may first be attached to the therapeutic agent, followed by attachment of the SAM molecule to the medical device, followed by attachment of the SAM molecule to the linker-therapeutic agent complex. Alternatively, the SAM molecule may first be attached to the linker-therapeutic agent complex, followed by attachment of the SAM molecule-linker-therapeutic agent complex to the medical device, assuming the complex maintains its ability to function as a SAM molecule, as set forth above.

5. Release of Therapeutic Agent

In certain embodiments of the present invention, the therapeutic agent is capable of releasing from the linker. Release can be by any mechanism known to those of ordinary skill in the art. For example, the therapeutic agent can be released by hydrolysis. Thus, for example, the linker may be attached to the therapeutic agent by an ester linkage, wherein the ester linkage is capable of releasing the therapeutic agent by acid hydrolysis. In this manner, the release of carboxyl-containing therapeutic agents can be controlled. The linker may be a bridging diol-linker, wherein the electron donating or withdrawing properties of substituents on the carbon atoms alpha to the hydroxyl groups will control the acid-labile properties of ester derivatives. The linker may be based on ethylene glycol, which will link alkyl carboxylic acid moieties of SAM constituents with a carboxyl group of a therapeutic agent. Modification of the acid-labile nature of the bridging linker will enable control of the release rate of therapeutic agents taking into consideration the expected pH of the microenvironment of the target site of the medical device.

The linker-therapeutic agent attachment can be designed to undergo hydrolysis upon implantation of the medical device in the body. The linker-therapeutic agent attachment can be designed to undergo hydrolysis in a pH-dependent manner, such as at physiologic pH, or in a temperature-dependent manner, such as at physiologic temperature. In other embodiments, the linker-therapeutic agent can be designed to release the therapeutic agent upon interaction of the therapeutic agent with a second agent, such as an agent that is intravenously administered to the subject following implantation of the medical device in the subject.

### C. THERAPEUTIC AGENTS

The term “therapeutic agent” is intended to refer to a chemical entity which is capable of providing a desired therapeutic effect when administered to a subject. The therapeutic effect can be treatment of a disease or prevention of a disease. The term “therapeutic agent” should be read to include synthetic compounds, natural products and macromolecular entities such as a peptide, polypeptide, protein, an enzyme, an antibody, a DNA molecule, an RNA molecule, or a small molecule. The term “therapeutic agent” is meant to refer to that compound whether it is in a crude mixture or purified and isolated. In certain embodiments of the present invention, the medical devices and methods may involve more than one type of therapeutic agent.

The therapeutic agent can be any therapeutic agent known to those of ordinary skill in the art. Representative examples of therapeutic agents are discussed in greater detail as follows:

1. Anticancer Agents and Antiproliferative Agents

An anticancer agent is defined herein to refer to an agent that is known or suspected to be of benefit in the treatment or prevention of cancer. An antiproliferative agent is defined herein to refer to an agent that is known or suspected to be of benefit in the treatment or prevention of a disease associated with an abnormal proliferation of cells or tissue. Thus, for example, while an anticancer agent is an antiproliferative agent, antiproliferative agents include other classes of agents that can be applied in the treatment of noncancerous conditions, such as cardiovascular stent restenosis following implantation for treatment of cardiovascular disease.

Examples of anticancer agents include 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, paclitaxel, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most of these agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

In certain preferred embodiments, the antiproliferative agent is an anti-restenotic agent. Anti-restenotic agents in cardiovascular disease are a broad spectrum of agents that interfere with migration and proliferation of smooth muscle cells at a site of stent-induced vessel injury. These agents include anti-inflammatory agents (including steroids, such as prednisolone, dexamethasone, methylprednisolone, etc.), immunosuppressive agents (such as sirolimus [rapamycin], tacrolimus, everolimus, ABT-578, biolimus-A9 and temsirolimus), and anti-mitotic agents such as paclitaxel and docetaxel.

2. Hormones

In certain embodiments of the present invention, the therapeutic agent is a hormone. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

3. Anesthetic Agents

An anesthetic is defined herein to refer to an agent that causes loss of sensation in a subject with or without the loss of consciousness. The loss of sensation can be local or general. Examples of local anesthetic agents include lidocaine, articaine, ultracaine, carticaine, benzocaine, amethocaine, bupivocaine, chloprocaine hydrochloride, etidocaine hydrochloride, diphenylhydramine, mepivacaine hydrochloride, and prilocaine. One of ordinary skill in the art would be familiar with these and other anesthetic agents that can be applied in the present invention.

4. Vasodilators

A vasodilator is defined herein to refer to an agent that causes dilation of a blood vessel in a subject following administration of the agent to the subject. Indications include cardiovascular disease, such as angina pectoris, aortic regurgitation, chronic heart failure, and myocardial infarction, chronic kidney disease, and migraine headaches. Exemplary vasodilators include calcium channel blockers such as amlodipine, diltiazem, nifedipine, nisoldipine, and verapamil. Others include papaverine, cilostazol, and nitroglycerin.

5. Anticoagulants and Anti-Platelet Agents

An anticoagulant is defined herein to refer to an agent that prevents or retards the clotting of blood. An example of an anticoagulant is an anti-platelet agent. An anti-platelet agent is defined herein to refer to an agent that prevents or retards the clotting of blood by affecting platelet structure or function. Anticoagulants are well-known to those of ordinary skill in the art. Examples of anticoagulants include warfarin, dicoumarol, and heparin.

6. Anti-Inflammatory Agents

An anti-inflammatory agent is defined herein to refer to an agent that is known or suspected to be of benefit in the treatment or prevention of inflammation in a subject. Corticosteroids are a major class of anti-inflammatory agent. The corticosteroids may be short, medium, or long acting, and may be delivered in a variety of methods. A non-limiting list of corticosteroids contemplated in the present invention include the oral corticosteroids such as: cortisone, hydrocortisone, prednisone, and dexamethasone.

Another major class of anti-inflammatory agents are non-steroidal anti-inflammatory agents. Non-steroidal anti-inflammatory agents include a class of drugs used in the treatment of inflammation and pain. The exact mode of action of this class of drugs is unknown. Examples of members of this class of agents include, but are not limited to, ibuprofen, ketoprofen, flurbiprofen, nabumetone, piroxicam, naproxen, diclofenac, indomethacin, sulindac, tolmetin, etodolac, flufenamic acid, diflunisal, oxaprozin, rofecoxib, and celecoxib. One of ordinary skill in the art would be familiar with these agents. Included in this category are salicylates and derivates of salicylates, such as acetyl salicylic acid, sodium salicylate, choline salicylate, choline magnesium salicylate and diflunisal.

Other anti-inflammatory agents include anti-rheumatic agents, such as gold salts (e.g., gold sodium thiomalate, aurothioglucose, and auranofin), anti-rheumatic agents (e.g., chloroquine, hydroxychloroquine, and penicillamine), antihistamines (e.g., diphenhydramine, chlorpheniramine, clemastine, hydroxyzine, and triprolidine), and immunosuppressive agents (e.g., methotrexate, mechlorethamine, cyclophosphamide, chlorambucil, cyclosporine, and azathioprine). Other immunosuppressive agent contemplated by the present invention is tacrolimus and everolimus. Tacrolimus suppresses interleukin-2 production associated with T-cell activation, inhibits differentiation and proliferation of cytotoxic T cells. Today, it is recognized worldwide as the cornerstone of immunosuppressant therapy. One of ordinary skill in the art would be familiar with these agents, and other members of this class of agents, as well as the mechanism of actions of these agents and indications for use of these agents.

7. Antibiotics and Antifungals

An antibiotic is defined herein to refer to a therapeutic agent that is known or suspected to be of benefit in the treatment or prevention of an infection by microorganisms in a subject. The infection may be an infection due to antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof. In certain preferred embodiments, the antibiotic is cefazolin.

One type of antibiotic is an antiseptic. An antiseptic is defined herein to refer to an agent used for preventing infection following an injury, such as by killing bacteria. Exemplary antiseptics include alcohols, chlorhexidine, chlorine, hexachlorophene, and iodophors.

An antifungal agent is herein defined to refer to a therapeutic agent that is known or suspected to be of benefit in the treatment or prevention of a fungal infection in a subject. Exemplary antifungal agents include fluconazole, itraconazole, amphotericin B, ketoconazole, and clotrimazole. One of ordinary skill in the art would be familiar with these and other antifungal agents.

8. Analgesics and Opiates

An analgesic is defined herein to refer to an agent that decreases the sensitivity of a subject to pain or prevents pain in a subject. Analgesic agents are well-known to those of ordinary skill in the art. Examples of this broad class of agents includes centrally acting narcotic agents, such as opioids. An opioid is any agent that binds to opioid receptors. Opioid receptors are found principally in the central nervous system and gastrointestinal tract. There are four broad classes of opioids: endogenous opioid peptides, produced in the body; opium alkaloids, such as morphine (the prototypical opioid) and codeine; semi-synthetic opioids such as heroin and oxycodone; and fully synthetic opioids such as pethidine and methadone that have structures unrelated to the opium alkaloids. Also contemplated are man-made narcotics, such as fentanyl and fentanyl derivatives.

Another broad class of analgesic is the peripherally acting analgesics. Examples of peripherally acting analgesics including aspirin, acetaminophen, and ibuprofen. One of ordinary skill in the art would be familiar with this broad class of agents.

9. Other

Other therapeutic agents include those agents that belong to more than one of the above classes of agents. For example, sirolimus (Rapamycin) is a triene macrolide antibiotic, which demonstrates anti-fungal, anti-inflammatory, anti-tumor and immunosuppressive properties. Rapamycin has been shown to block T-cell activation and proliferation, as well as, the activation of p70 S6 kinase and exhibits strong binding to FK-506 binding proteins. Rapamycin also inhibits the activity of the protein, mTOR, (mammalian target of rapamycin) which functions in a signaling pathway to promote tumor growth. Rapamycin binds to a receptor protein (FKBP12) and the rapamycin/FKB12 complex then binds to mTOR and prevents interaction of mTOR with target proteins in this signaling pathway.

### D. MEDICAL DEVICES

1. Definitions

A “medical device” is defined herein to refer to an instrument, apparatus, implement, machine, contrivance, implant, in vitro reagent, or other similar or related article, including a component part, or accessory which is: (a) intended for use in the diagnosis of disease or other conditions, or in the cure, mitigation, treatment, or prevention of disease, in a subject, or (b) intended to affect the structure or any function of the body of a subject. The subject may be a mammal, such as a human or a laboratory animal. The medical device may be suitable for implantation in a subject or application on a surface of the subject.

One example of a medical device is a stent. A “stent” is defined herein to refer to a medical device that is inserted into a vessel or passage to keep it open or to support a bodily orifice or cavity. For example, the stent may be a vascular stent that is inserted into a blood vessel to keep the blood vessel patent. Examples of a vascular stents include coronary stents (discussed in greater detail in the specification below) and arterial stents. Additional examples of stents include GI stents, pulmonary stents, and ureteral stent.

Other examples of medical devices include a valves, synthetic grafts, metal plates, musculoskeletal fixation systems, pins, artificial joints (e.g., temporal mandibular joints), dental implants, ocular implants, neural implants, artificial hearts, artificial organs, or an implant in contact with body fluids.

In some embodiments, the medical device is suitable for application to a body surface of a subject, such as a skin surface, a mucosal surface, a wound surface, a surface of a hollow viscus, or a tumor surface. For example, the medical device may be a stent designed to immobilize a skin graft following placement.

2. Coronary Stents

Stents are small, expandable, metal devices inserted by a catheter into a narrowed artery after the angioplasty procedure is complete (reviewed in Jost, 1998). Stents are left in place to help keep the artery from closing again (restenosis). Stents may be classified based on their pattern of metal construction (slotted tube, coil or mesh) or type of stent delivery system (self-expandable or balloon-expandable).

Examples of types of coronary stents include original slotted tube stents, second generation tubular stents, self-expanding stents, coil stents, and modular zigzag stents

In general, slotted-tube systems, characterized by the PS stent, are characterized by high vessel surface area coverage, high radial strength and consistent circumferential deployment pattern. Coil stents provide for greater flexibility, conformability to the target vessel tortuosity, and access to side-branches but have significant intrinsic recoil. Mesh-design stents, found in many of the second generation tubular stents, are a hybrid of slotted tube and coil features. They possess the sizing strategies and deployment mechanics of slotted tube stents; and flexibility, conformability and side-branch access of the coil stents.

Some stents are of a slotted-tube design in a repeating sine wave pattern without articulation sites. The stent may or may not be flexible.

Some stents are designed for bifurcation lesions. Other stents are covered by a thin layer of material. For example, the stent may be constructed with a sandwich technique whereby an ultrathin layer of expandable PTFE is placed between two stents with reduced strut thickness. The use of a segment of autologous vascular tissue for stent cover has also been (Stefanadis et al., 1996). For example, a segment of the cephalic or ulnar artery is harvested and crimped onto the stent for deployment.

The stent may also be a platform for the delivery of radiation to the vessel wall to help combat restenosis. Effective doses of radioactivity can be delivered to all levels of the vessel wall from stent-bound radioactive sources. Most of the current interest has been focused on b-emitting stents because of the initial success in reducing neointimal proliferation in animal studies (Laird et al., 1996; Hehrlein et al., 1996).

Exemplary coronary stents are set forth in U.S. Pat. No. 6,893,413, U.S. Pat. No. 6,875,227, U.S. Pat. No. 6,562,066, U.S. Pat. No. 6,532,380, U.S. Pat. No. 6,398,804, U.S. Pat. No. 6,287,332, U.S. Pat. No. 6,068,656, U.S. Pat. No. 6,053,942, U.S. Pat. No. 6,017,365, U.S. Pat. No. 5,938,695, U.S. Pat. No. 5,897,588, U.S. Patent Application Pub. No. 20040116999, U.S. Patent Application Pub. No. 30020130719, U.S. Patent Application Pub. No. 20030130611, U.S. Patent Application Pub. No. 20030125798, and U.S. Patent Application Pub. No. 20030114921, each of which is herein incorporated by reference in its entirety.

2. Composition

A medical device can be composed of any material or mixture of materials known to those of ordinary skill in the art. Examples of such materials include stainless steel (e.g., 316L SS and 304 SS), titanium, tantalum, cobalt, chromium, gold, silver, triclosan, platinum, a polymer, a polymer derivative, a copolymer, a multi-component copolymer, glass, pyrolytic carbon, alumina, zirconia, titania, graphite, or a ceramic. The medical device may be composed on a mixture of metals (i.e., an alloy) selected from the group consisting of stainless steel, titanium, tantalum, cobalt, chromium, gold, silver, platinum. For example, the alloy may be Nitinol or niobium-zirconium. Other examples of materials include polymers, such as poly(ethylene glycol), poly(caprolactone), poly(hydroxyethyl methacrylate), poly (lactic acid), poly(ethylene), poly(glycolic acid), poly(styrene), a poly(anhydride), a poly (urethane), a poly(carbamate), a poly(ester), or a derivative of any of these polymers.

The polymer may be a polymer composed of more than one type of monomer. For example, the polymer may be a terpolymer. Alternatively the medical device may be comprised of more than one type of polymer, such as a polymer blend.

In some embodiments, the medical device is composed of a resorbable polymer, such as polytetramethyleneoxide (PTMO), aliphatic polycarbonate based oligomers, hydroxyl-terminated or amino-terminated oligomers with linear or branched aliphatic backbone structure typified by polyisoprene, polybutadiene, polyisobutylene, or carbinol terminated polydimethylsiloxanes (PDMS). Resorbable polymers are addressed in greater detail in U.S. Patent Application Pub. No. 20050060022, which is herein incorporated by reference in its entirety.

The medical device may be composed in whole or in part of natural materials, such as various tissues that are harvested, extracted, cultured or otherwise obtained either directly or indirectly from human and animal physiologies. These are discussed in greater detail in U.S. Patent Application Pub. No. 20050085898, which is herein incorporated by reference in its entirety.

The medical device may be comprised of radiopaque material, such as radiopaque markers. Radiolucent medical devices with radiopaque markers are discussed in greater detail in U.S. Patent Application Pub. No. 20050084515 and U.S. Patent Application Pub. No. 20050085895, which are herein incorporated by reference in their entirety.

3. Fabrication of Medical Device with a Self-Assembled Monolayer

Methods of attachment of a self-assembled monolayer molecule to a medical device are discussed in greater detail elsewhere in this specification. In certain embodiment, a SAM may be formed on a single surface of a medical device. In other embodiments, a SAM may be formed on more than one surface of a medical device. In further embodiments, a SAM is formed on only a portion of a surface of a medical device. As discussed elsewhere in this specification, any method known to those of ordinary skill in the art can be used to attach a self-assembled monolayer molecule to the medical device.

In certain embodiments, a therapeutic agent is attached to only a fraction of the self-assembled monolayer molecules forming the SAM. Thus, for example, the amount of therapeutic agent that is attached to the medical device can be varied on the surface of the medical device. In this manner, the medical device can be tailored to include therapeutic agent on surfaces or areas of the medical device where the therapeutic agent is needed.

For example, in some medical devices having a tubular wall, all of the surfaces of the medical device or portions thereof may not need to be coated with a SAM, or may not need to be coated with a coating comprising a therapeutic agent. For instance, the inner surface of a stent does not have to be coated with a coating containing a biologically active material when the biologically active material is intended to be delivered to a body lumen wall, which only directly contacts the outer surface of the stent. The inner surface of the stent does not come in direct contact with the body lumen wall and does not apply the biologically active material to the body lumen wall. On the other hand, if the biologically active material is intended to be delivered to a body fluid rather than a body lumen wall, then the coating containing the biologically active material should be placed on the inner surface of the stent wall but is not needed on the outer surface.

In other embodiments, the release profile of a therapeutic agent can be optimized by varying the amount of therapeutic agent that is bound to an axis of the medical device. For example, the amount of therapeutic agent along the longitudinal axis of a tubular stent can be varied. For example, in stents, the amount of bound therapeutic agent may be preferably increased at the end sections of the stent as compared to the middle portion to reduce a risk of restenosis caused at the end sections.

In addition, SAMs on different portions of the tubular wall may require different physical properties. For example, an expandable stent must be put in its unexpanded state or “crimped” before it is delivered to a body lumen. Thus, the coating on portions of the stent which contact each other in the stent's crimping state must not stick to each other and cause damage. In the case of a balloon expandable stent, the inner surface of the stent that contacts the balloon must not stick to the balloon during expansion. On the other hand, it is desirable to provide a relatively soft or “sticky” coating on the outer surface because it comes in direct contact with a body lumen wall.

### E. SUBJECTS AND METHODS OF ADMINISTRATION

1. Subjects

As set forth herein, the subject can be any subject, such as an avian species or a mammal. For example, the mammal can be a human or a laboratory animal. In certain particular embodiments, the human is a patient with a disease that requires treatment with a particular therapeutic agent or agents, or a human at risk of developing a particular disease or condition for which preventive therapy with a particular agent is indicated.

In certain embodiments, the patient is a patient with cardiovascular disease, hyperproliferative disease, coronary artery disease, valvular heart disease, heart failure, peripheral vascular disease, uereteral obstruction, bile duct obstruction, broncial or tracheal obstruction, arthritis, degenerative joint disease, fractures, arthritis, fractures, degenerative joint disease, cancers, broken bones, induction system disease cardiac arrhymthous, or a person at risk of sudden cardiac disease.

The patient may be in need of surgical therapy with implantation or application of a medical device for treatment or prevention of any disease. For example, the disease may be cardiovascular disease, hyperproliferative disease, a burn, coronary artery disease, valvular heart disease, heart failure, peripheral vascular disease, uereteral obstruction, bile duct obstruction, broncial or tracheal obstruction, arthritis, degenerative joint disease, fractures, arthritis, fractures, coronary artery disease, valvular heart disease, heart failure, peripheral vascular disease, uereteral obstruction, bile duct obstruction, broncial or tracheal obstruction, arthritis, degenerative joint disease, fractures, arthritis, fractures, cancers, broken bones, induction system disease cardiac arrhymthias, or sudden cardiac disease.

2. Dosage

An effective amount of the therapeutic or preventive agent is determined based on the intended goal. For example, the therapeutic goal may be prevention of restenosis in a stent. The quantity of therapeutic agent to be administered, depends on the subject to be treated, the state of the subject, protection desired, the design of the medical device, and the expected location of the medical device in the subject. Precise amounts of the therapeutic composition also depend on the judgment of the practitioner and are peculiar to each individual.

For various approaches, delayed release formulations could be used that provide limited but constant amounts of the therapeutic agent over an extended period of time. For example, the medical device can be designed to promote delayed release of the therapeutic agent by incorporating a polymer into the SAM which overlies the therapeutic agent to delay release of the therapeutic agent following implantation of the medical device into the system. Alternatively, the medical device could be designed to incorporate more than one type of SAM, such that one type of SAM sterically interferes with release of therapeutic agent from a second, small type of SAM following implantation of the medical device into a subject. In some embodiments, the SAM may be coated with a material that covers or interacts with the therapeutic agent, such that delayed release results following implantation of the medical device into a subject. One of ordinary skill in the art would be familiar with approaches to promote delayed release of therapeutic agents.

In some embodiments, it may be possible to follow release of a therapeutic agent from a medical device by incorporating a radiolabel that is designed to release following release of the therapeutic agent following implantation or contact of the device with the subject. One of ordinary skill in the art would be familiar with incorporation of radiolabels, and methods of imaging radiolabels.

3. Administration

As set forth above, the medical device can be any medical device known to those of ordinary skill in the art. Examples are set forth above. One of ordinary skill in the art would be familiar with methods of implantation of a medical device in a subject, or methods of application of a medical device on the surface of a subject. Particular modification of these methods may be required in view of the therapeutic agent attachment to the medical device, such as minimizing handling of the surface of the medical device comprising the therapeutic agent during implantation.

4. Monitoring

Monitoring of therapy with medical devices of the present invention will be by any method known to those of ordinary skill in the art. For example, following coronary stent implantation, monitoring of the release of therapeutic agent may be by measurement of vascular patency by any method known to those of ordinary skill in the art (e.g., coronary arteriography). Monitoring release of a therapeutic agent may include measurement of blood level of the therapeutic agent, or measurement of a blood parameter that provides an indication of level of therapeutic agent (e.g., measurement of platelet function following administration of an anti-platelet agent). Medical device placement can be monitored radiographically or by any other method known to those of ordinary skill in the art.

5. Other

The present invention further contemplates situations in which a medical device of the present invention comprises more than one therapeutic agent. The present invention further contemplates situations wherein a subject may require implantation with more than one of the medical devices set forth herein (e.g., stent placement in two different vessels).

### F. SECONDARY THERAPIES

1. Secondary Therapy in General

Certain aspects of the present invention pertain to methods of administering a therapeutic agent to a subject that involve administration of one or more secondary forms of therapy. These medical devices set forth herein can be applied in the prevention or treatment of any disease wherein the therapeutic agent and medical device is known or suspected to be of benefit.

For example, as set forth above, the disease or health-related condition to be treated or prevented may be a hyperproliferative disease or a cardiovascular disease. The medical device with attached therapeutic agent may be administered along with another agent or therapeutic method directed to the disease to be prevented or treated. For example, the secondary form of therapy may precede, follow, or be concurrent with other therapies for cardiovascular disease, such as angioplasty or administration of on oral vasodilator.

Therapy using the medical devices set forth herein will follow general protocols for the administration of therapeutic agents, and will take into account other parameters, including, but not limited to, other medical conditions of the patient and other therapies that the patient is receiving. It is expected that the treatment cycles of the secondary therapy may be repeated as necessary.

Treatment with the medical device of the present invention may precede or follow the other therapy method by intervals ranging from minutes to weeks. In embodiments where one or more additional therapeutic agents is administered, one would generally ensure that a significant period of time did not expire between the time of each delivery, such that the agents would still be able to exert an advantageously combined effect on the subject. For example, it is contemplated that one may administer two, three, four or more doses of a secondary agent substantially simultaneously (i.e., within less than about a minute) with the compositions of the present invention. In other aspects, a secondary therapeutic agent or method may be administered within about 1 minute to about 48 hours or more prior to and/or after implantation or application of the medical device, or prior to and/or after any amount of time not set forth herein. In certain other embodiments, the medical device may be administered within of from about 1 day to about 21 days prior to and/or after administering another therapeutic modality, such as surgery, radiation therapy, immunotherapy, gene therapy, or medical therapy. In some situations, it may be desirable to extend the time period for treatment significantly, however, where several weeks (e.g., about 1 to 8 weeks or more) lapse between the respective administrations. One of ordinary skill in the art would be familiar with designing protocols for administration of multiple therapeutic modalities to a subject.

2. Exemplary Secondary Therapies

a. Cardiovascular Disease

Cardiovascular disease is a very common cause of morbidity and mortality in Americans. Heart disease is the leading cause of death for all racial and ethnic groups in the U.S. More than half of persons who die each year of heart disease are women. Exemplary cardiovascular diseases include acute myocardial infarction, atherosclerosis, and congestive heart failure.

There are many forms of therapy of cardiovascular disease, including pharmacological therapies, dietary interventions, and more invasive forms of therapy, including angioplasty and cardiovascular surgery.

Over the counter aspirin (might be beneficial for reducing the risk of future heart attacks. Use of prescription medications is directed toward any underlying causes. Drugs used may include ACE inhibitors, such as captopril, enalopril, and lisinopril; beta blockers such as atenolol, meoprolol, and propranol; and the combination of hydralazine and isosorbide dinitrate. Other medications often prescribed include the blood thinner warfarin, digoxin, nitroglycerin, and diuretics, such as hydrochlorothiazide and furosemide.

Surgical treatments, such as angioplasty, bypass surgery, valve replacement, pacemaker installation, and heart transplantation, may be recommended for severe cases. Individuals with cardiovascular disease are strongly encouraged to stop smoking.

b. Hyperproliferative Disease

In certain embodiments of the present invention, the subject to be treated is a patient with a hyperproliferative disease, such as cancer. Administration of the therapeutic medical devices of the present invention to a patient will follow general protocols for the administration of chemotherapeutics, taking into account the toxicity, if any, of these agents. It is expected that the treatment cycles would be repeated as necessary. It also is contemplated that various standard therapies may be applied in combination with the therapeutic medical devices set forth herein. These therapies include but are not limited to chemotherapy, radiotherapy, immunotherapy, gene therapy and surgery. One of ordinary skill in the art would be familiar with these therapeutic modalities.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Formation of Self-Assembled Monolayers

Formation of SAMs on titanium and 316L stainless steel and confirmation thereof. Studies were conducted to investigate formation of SAMs on titanium and 316L stainless steel, with the possibility of using either material for potential medical devices such as stents.

Formation and confirmation of functional SAMs on 316L stainless steel (SS). 316L SS plates (20 mm×20 mm×2 mm) were obtained from ESPI Corp Inc, Ashland, Oreg. The samples were polished by using a Handimet Grinder polishing machine with 4 types of grit papers (240, 320, 400, and 600 grit papers). The roughness of the polished 316L SS plates was measured as 0.2±0.1 pm. The samples were cleaned chemically as follows: ultrasonicated in 70 percent ethanol for 10 minutes, followed by ultrasonic cleaning in acetone for 10 minutes and ultrasonication in 40 percent nitric acid for 10 minutes. This treatment is hereafter referred to as the “chemical treatment.” To improve the surface activation for deposition of SAMs, the SS plates were subjected to glow discharge gas plasma (GDGP) treatment in a radio frequency glow discharge system (Harrick Scientific, NJ) for 4 minutes in an oxygen environment under reduced pressure. Then the plates were immediately dipped in amphiphile solutions of either 1-dodecane thiol (CH₃ SAM), 11-mercaptol-undecanol (OH-SAM) or 16-mercaptohexadecanoic acid (COOH SAM) to form respective SAMs on 316L SS (preparative details of the amphiphile solution described below). After rinsing and washing the samples with ethanol and ultrapure water, the samples were characterized using contact angle measurements and XPS. Table 2 shows significant differences in contact angle between bare SS after GDGP (less than 3 deg) and those of the functional SAM formed.

Preparation of amphiphile solutions of 16-mercaptohexadecanoic acid. 2 mM solution of 16-mercaptohexadecanoic acid (HSCH₂—(CH₂)CH₂—COOH), in ethanol/water/acetic acid was prepared by dissolving 0.058 g of 16-mercaptohexadecanoic acid in 100 ml of ethanol/water/acetic acid (85/10/5 v/v/v). Preparation of Amphiphile Solutions of 11-mercaptol-undecanol: a 10 mM of solution of 11-Mercaptol-undecanol [HOCH₂(CH₂)₉CH₂SH] in ethanol was prepared by dissolving 0.204 g of 11-mercaptol-undecanol in 100 ml of ethanol.

Preparation of amphiphile solutions of 1-dodecane thiol. A 10 mM of solution of 1-dodecane thiol [CH₃(CH₂)₁₀CH₂SH] in ethanol was prepared by dissolving 0.218 g of 11-mercaptol-undecanol in 100 ml of ethanol.

TABLE 2
Static contact angle values of treated SS

| | Static Contact | Standard |
|---|---|---|
| Monolayer | Angle | deviation |
| Bare SS | 94.3° | 4.0 |
| Bare SS after plasma | 3°< | — |
| treatment | | |
| CH₃-SAMs | 69° | 5.1 |
| OH-SAMs | 45.4° | 12.8 |
| COOH-SAMs | 55.4° | 21.0 |

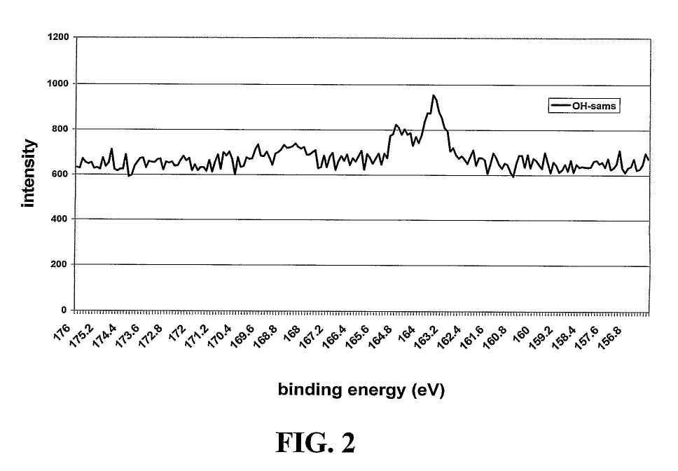

The contact angle for CH3, HO, COOH SAMs were in agreement to previously reported hydrophobic and hydrophilic SAMs on 316L SS (Shustak et al, 2004; Ruan et al., 2002). XPS analysis of OH-SAMs (FIG. 2) shows peaks at 162 ev, which represents the peak of the metal thiolate. The identity of adsorbate as the metal thiolate rather as an oxidized sulphur is clear due to lack of peaks for sulfinates and sulphonates (binding energy 169 eV) (Ruan et al., 2002). The contact angle and XPS data confirms formation of a stable thiolate SAM on SS.

Formation and confirmation of hydroxyl acid SAMs on titanium. Ti-6AI-4V plates—grade 5 (20 mm×20 mm×2 mm) were obtained from TIMET Corp. The Ti-6AI-4V plates were polished, chemically treated and subjected to GDGP treatment as described for 316L SS. Lastly, SAMs were attached to the samples as follows: 1 gram of 12-hydroxydodecanoic acid (12-HDDA) was dissolved in 100 ml of ethanol at 83° C. Ammonia solution was then added and the contents were placed in an ice bath until the temperature reached 10° C. The ammonium salt of 12-HDDA (NH₄-12-HDDA) was filtered and dried. 15 mg of this salt was dissolved in 100 ml of purified water. The Ti-6AI-4V samples were dipped in this amphiphile solution to form the SAMs. The contact angle increased very significantly after GDGP (3<deg) to (36.7±2.9) after SAM formation, which is typical for well defined hydrophilic SAMs (Tosatti et al., 2002), confirming the formation of OH-SAMs on the metal surface. This study confirms that SAMS can be successfully attached to 316L SS and Ti-6AI-4V surfaces.

### Example 2

### Development of Synthetic Methodologies for Coupling Therapeutic Agents to Metal Surfaces Via SAMs

Chemical synthetic methodologies for coupling therapeutic agents to the metal surface can follow two strategies (a) chemical modification and attachment of therapeutic agent after formation of SAMs (b) attachment of therapeutic agent-linker prior to assembly of SAM.

Biocatalysis, which involves the use of enzymes, microbes, and higher organisms to carry out chemical reactions, may serve as an alternate route for surface modification of SAMs. Biocatalysis is well established in the production of pharmaceuticals, food, agrochemicals, and fine chemicals. Use of enzymes in organic synthesis (Roberts, 2001) and polymer science (Gross et al., 2001) has been discussed elsewhere within comprehensive reviews. Use of enzymes for surface modification of SAMs on a metal surface offers distinct advantages: (1) development of methodologies of attachment of those therapeutic moieties on metal surface after assembly of SAMs, which, due to steric hindrance, may be difficult to achieve via chemical means; (2) elimination of the use of organic solvents by carrying out reactions in bulk (solvent less), or aqueous medium; and (3) use of mild reaction conditions (RT to 70° C.) ensuring structural integrity of the SAMs formed. Reported selectivity of enzyme reactions may provide spatial and topographical ordering of the surface.

The studies set forth herein focused on exploring several strategies simultaneously with the intent of ultimately identifying the best method for maximized drug attachment. Thus, the chemical strategies were explored on titanium surfaces, whereas the biocatalytic routes were pursued for 316L SS.

Attachment of therapeutic moieties on titanium. For proof of concept, aspirin was selected as a model drug as it consists of a carboxylic functional group which could be attached to the hydroxyl functional SAM on titanium prepared as described earlier in Example 1. Once established with aspirin, these reactions could be easily transposed to other drugs with similar functionalities. Therapeutic self-assembled monolayers (T-SAMs) were formed by two procedures: 1) AT-AS procedure—Aspirin was first attached (AT) to NH₄-12-HDDA and then monolayer assembled (AS) on Ti surface; and 2) AS-AT procedure—NH₄-12-HDDA SAM was first assembled on Ti and then the aspirin was attached.

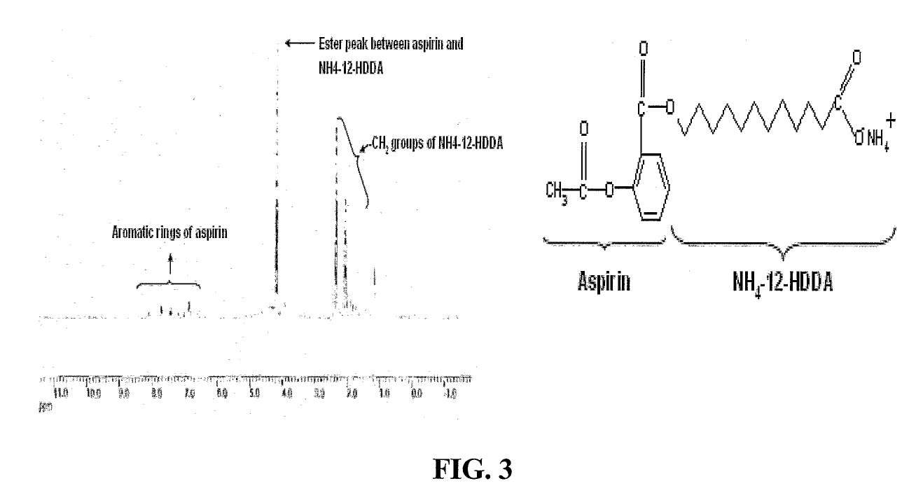

AT-AS procedure (attachment of aspirin followed by assembly). 40 ml of THF was taken in a 100 ml round bottom flask. 0.2 grams of NH₄-12-HDDA and 0.4 grams of aspirin were added to THF. Then, 250 pI of H₂60₄ was added. The mixture was refluxed at 65° C. for 24 hours; subsequently, the solvent was evaporated and oven dried 12 hours. The compound obtained, was characterized by ¹H NMR (FIG. 3) ¹H NMR spectra of aspirin attached NH₄-12-HDDA (Asp-NH₄-12-HDDA) is shown in FIG. 3. The strong ester peak at 4.2 ppm indicates the attachment of aspirin at the terminal —OH groups of NH₄-12-HDDA. The peaks at 7.1, 7.3, 7.6, and 7.9 ppm indicate the presence of aromatic rings of aspirin. The peaks at 1.3, 1.4, 1.5 and 2.3 ppm indicate the presence of —CH₂ groups of NH₄-12-HDDA. The amphiphile solution of Asp-NH₄-12-HDDA, for SAM attachment was prepared by dissolving 15 mg Asp-NH₄-12-HDDA in 5 ml of 70% ethanol, and then adjusting the volume to 100 ml by adding 95% of ethanol. Seven Ti-plates were dipped in the above prepared amphiphile solution for 48 hours. The samples were taken out and rinsed in ethanol and dd-water prior to contact angle measurements and XPS analysis.

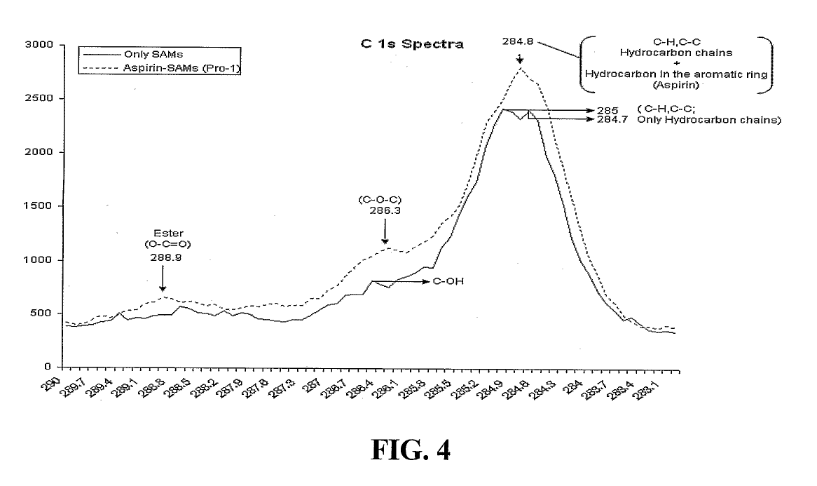

FIG. 4 shows the Cl XPS spectra of the above compound after its assembly on titanium. The C 1 s signals at higher binding energies, 288.9 eV and 286.3 eV, can be assigned to ester and C—O—C bonds of Asp-NH₄-12-HDDA on Ti, which is completely absent in control samples (NH₄-12-HDDA on Ti). Also, when compared with control samples, there is a marked increase in hydrocarbons intensities (284.8 eV) because of the aromatic carbon atoms of aspirin. The above interpretations of C 1s signals are in excellent agreement with increased % atomic concentrations of carbon atoms in FIG. 4. The % of carbon atoms has been increased from 33.36% to 40.14% for NH₄-12-HDDA and Asp-NH₄-12-HDDA, respectively.

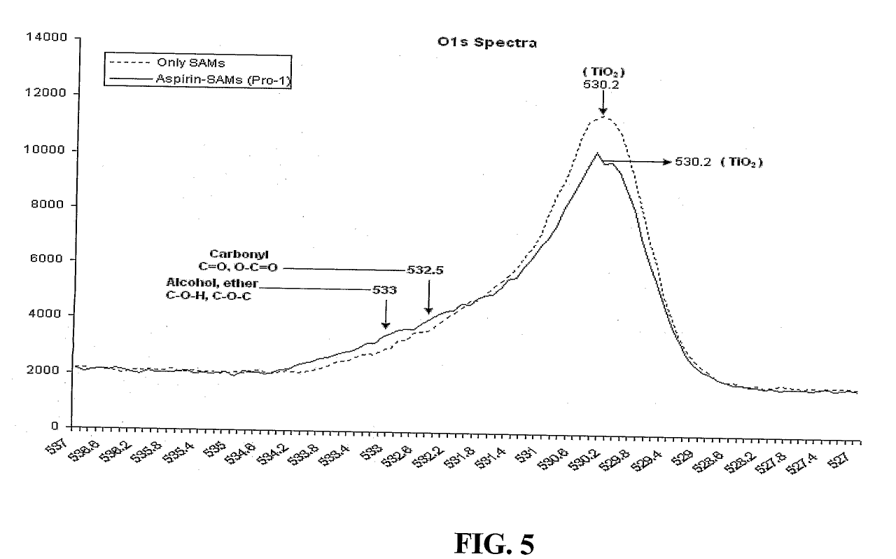

The peak at 530 eV in FIG. 5 (XPS 0 1s spectra) is typical for TiO₂. The decrease in TiO₂ intensity at 530 eV and the formation of carbonyl (C═O, O—C═O) and alcohollether (C—O—H, C—O—C) peaks at 532.5 eV and 533 eV suggests surface coverage of Asp-NH₄-12-HDDA on Ti, which is further supported by the % of oxygen atoms has been decreased from 64.77% to 58.17% for NH₄-12-HDDA and Asp-NH₄-12-HDDA respectively.

The water contact angle of NH₄-12-HDDA on Ti (36.68°±2.9), shows presence of a hydrophilic surface, which is due to the presence of the hydroxyl terminated SAMs. After the formation of Asp-NH₄-12-HDDA, the contact angle has been increased to 41.2±6.3, because of the attached aspirin at the terminal group.

AS-AT procedure (assembly then attachment of aspirin). Functional SAM (OH-SAM) on Ti (as prepared in Example 1) was used as a precursor to attach therapeutic moieties on titanium as follows. 40 ml of THF was taken in a 100 ml round bottomed flask. 0.2 grams of aspirin and 250 μl of H2SO4 were added and stirred till dissolved in THF. NH4-12-HDDA self-assembled Ti plates were then dipped in the THF mixture and the flask was refluxed at 65° C. for 24 hours. Ti-plates were taken out, rinsed and washed in dd-water. The surfaces of Ti samples were investigated by XPS and contact angle measurements.

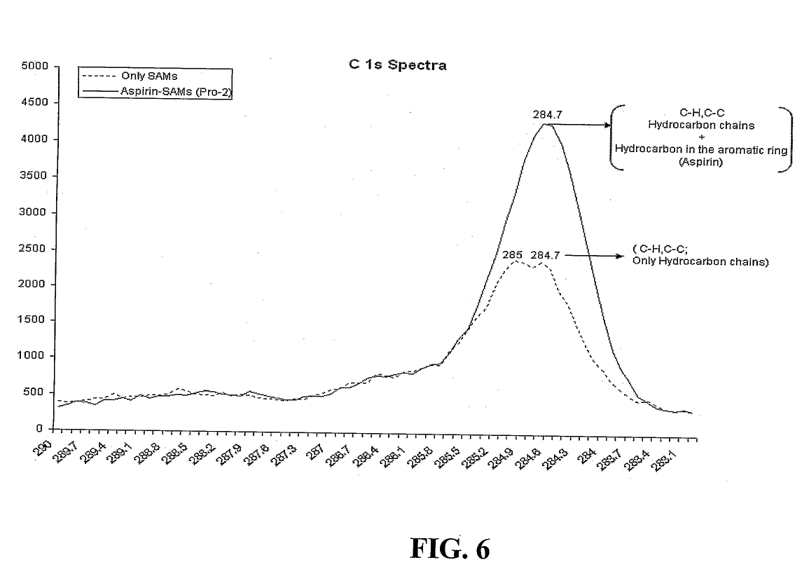

Comparing XPS spectral analysis with control samples (functional SAM), showed marked increase in intensity for hydrocarbons (284.7 eV) because of the aromatic carbon atoms of aspirin (FIG. 6). Although peaks of ester and carbonyl peaks were not visible in the XPS spectra for Asp-NH4-12-HDDA SAMs on Ti, the above increased intensity of hydrocarbons are in excellent agreement with % atomic concentrations of carbon atoms, which increased from 33.36% to 54.46% for NH₄-12-HDDA and Asp-NH₄-12-HDDA respectively for this AS-AT procedure. Further XPS analysis could possibly require deconvolution of peaks or higher scan intensity for all peaks to be visible.

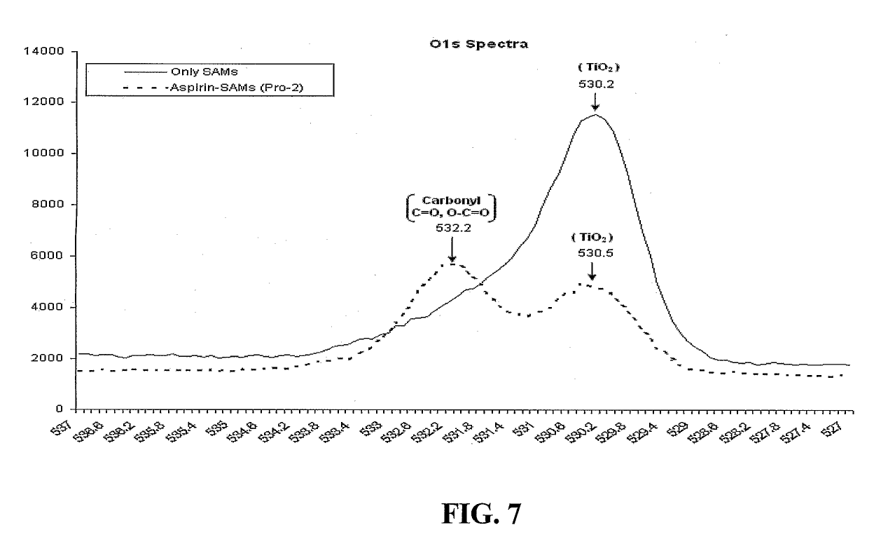

The peak at 530.2 eV in FIG. 7 (XPS 0 1s spectra) is typical for TiO₂. The decrease in TiO₂ intensity at 530.5 eV and the strong formation of carbonyl peaks at 532.2 eV in 0 1 spectra indicates large surface coverage of Asp-NH₄-12-HDDA formation on Ti, which is further supported by decreased % of oxygen atoms concentration from 64.77% to 58.17% for NH₄-12-HDDA and Asp-NH₄-12-HDDA respectively. The water contact angle of NH₄-12-HDDA on Ti (36.68°±2.9) shows presence of hydrophilic nature of the surface because of the hydroxyl terminated SAMs. After attachment of aspirin, (Asp-NH₄-12-HDDA), the contact angle increased (38.4±11.2) because of the attached aspirin at the terminal group, although this preliminary study did not show statistically significant differences.

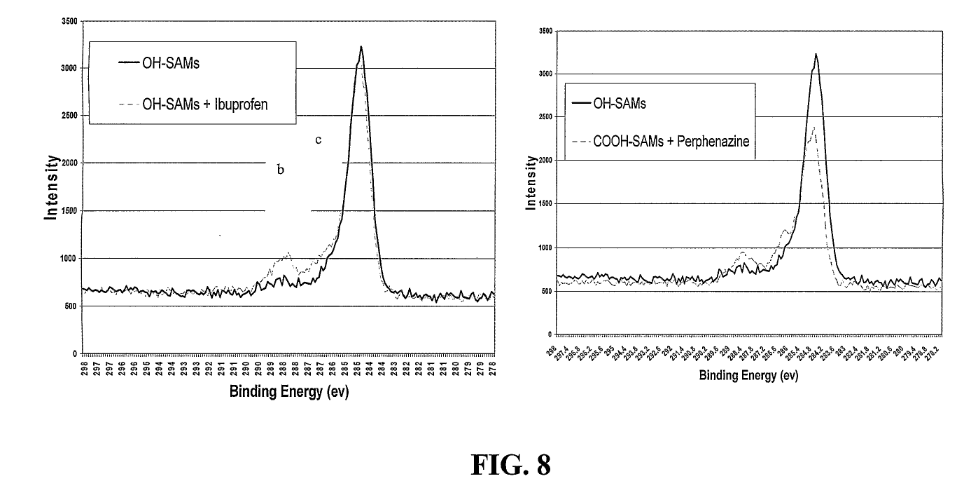

Biocatalysis-attachment of therapeutic moieties on 316L SS. This was used as an alternate route to attach chemical moieties to the functional 316L SS (as prepared in Example 1). Functional SAMs (OH-SAM or COOHSAM) on 316L SS prepared using 11-mercaptol-undecanol (OH-SAM) or 16-mercaptohexadecanoic acid (COOH-SAM) were used as precursors to attach therapeutic moieties on steel as follows: samples of the functional SAMs on 316L SS were taken in a beaker containing 10 ml toluene to which 50 mg of the drug (perphenazine for COOH-SAMs and ibuprofen for OH-SAMs) were added. The drugs were selected based on their having the appropriate functionalities: ibuprofen has a —COOH functional group that could be attached to the OH-SAM, whereas perphenazine has a —OH functional group that could be attached to the COOH-SAM. These drugs were selected for proof of concept; however, similar reactions could be easily transposed to other drugs with similar functionalities. Finally, 10 mg of novozyme-435 was added as a biocatalyst. Selection of novozyme was based on previous reports of it being the preferred biocatalyst for esterifications reactions (Mahapatro et al., 2004a). The beaker was covered with aluminum foil and was placed in a shaking water bath maintained at 60° C. for 5 hr. After 5 hr the steel plates were removed and washed and rinsed with ethanol, acetone and dd-water. These samples were then characterized using XPS (FIG. 8) and contact angle measurements (Table 3).

TABLE 3
Static contact angle values of treated SS

| | Static Contact | Standard |
|---|---|---|
| Monolayer | Angle | deviation |
| Bare SS after plasma | 3°< | — |
| treatment | | |
| OH-SAMs | 45.4° | 12.8 |
| OH-SAMs + ibuprofen | 74° | 22.6 |
| COOH-SAMs | 55.4° | 21.0 |
| COOH-SAMs + | 86.2° | 10.5 |
| perphenazine | | |

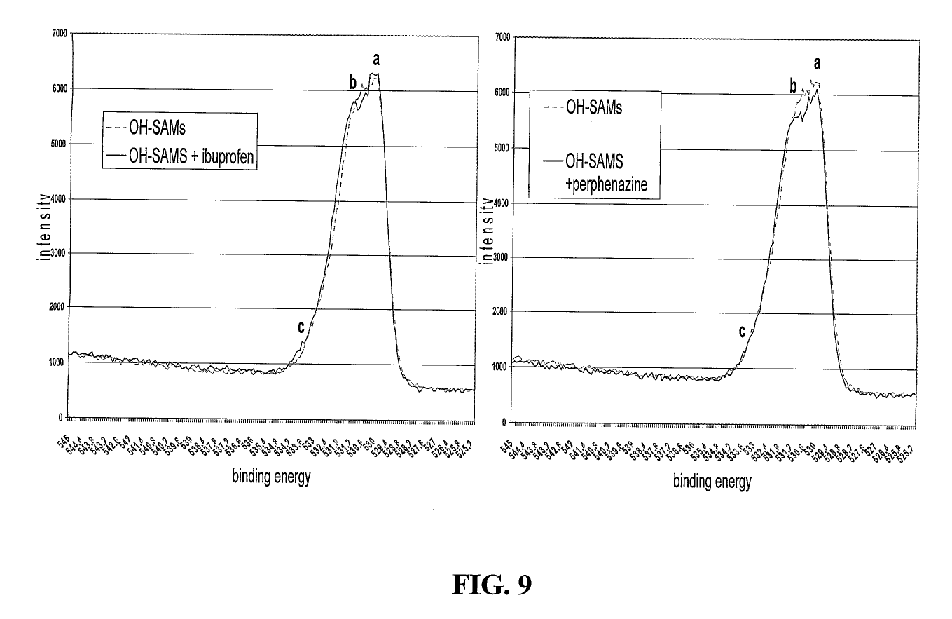

XPS analysis in the C region (FIG. 8) of samples after biocatalysis clearly indicates attachment of the therapeutic moiety. This is evident by the C=0 peak (present on ibuprofen) at 288 eV which is absent in the control OH-SAMs. Similarly, presence of ester C—O peak at 286.5 eV for the COOH-SAMs confirms drug attachment, as this peak would only evolve after esterifications and would be absent for the COOH-SAMs sample. XPS analysis in the O region (FIG. 9) shows marginal decrease for free OH (contributed by hydroxyl and carboxylate groups, binding energy O—H, 531.8 eV) after drug attachment, this would be expected as formation of ester bond would decrease the available free —OH. Higher scans would be required to resolve peaks clearly.

Confirmation of drug attachment is also supported by the contact angle measurement data (Table 3), which clearly shows increase in contact angle after drug attachment (45.4±12.8 for OH-SAM to 74±22.6 after drug attachment) and similarly for COOH SAMs.

### Example 3

### Surface Modification of Function Self-Assembled Monolayers (SAMs) on 316L Stainless Steel Via Lipase Catalysis

### Materials

316L SS Plates were obtained from ESPI Corp. Inc, Ashland, Oreg. 16-mercaptohexadecanoic acid, 11-mercapto-1-undecanol and Novozyme-435 were purchased from Aldrich Chemical Co. and used as received. Novozyme-435 consists of Candida Antartica Lipase B (CALB) physically adsorbed within the macroporous resin Lewatit VPOC 1600 (supplied by Bayer). Lewatit consists of poly(methylmethacrylate-co-butylmethacrylate), has a protein content of 0.1 w/w, surface area of 110-150 m²g⁻¹, and average pore diameter of 140-170 Å (Mahapatro et al., 2004). Organic solvents were all analytical grades and purchased from Aldrich Chemical Co.

### Characterization Methods

Contact Angle Measurements. Static contact angles were recorded using a VCA Optima S, surface analysis system. Droplet profiles were captured using a video and transferred to a computer for angle measurement. Contact angles were measured on both sides of the drop. Reported values are the average of 15 measurements, taken from 5 specimens, with 3 readings per specimen taken at different locations of the surface of the sample.

Fourier transform infrared spectroscopy (FTIR). FTIR was used to evaluate the structural and molecular composition of the SAMs. Samples were analyzed from 4000 cm⁻¹ to 500 cm⁻¹. FTIR spectra was acquired using a dry air purged Thermo Mattson Infinity Gold FTIR with an attenuated total internal reflectance accessory and a nitrogen cooled MCT detector. All spectra were obtained using p-polarized light incident on the substrate at an angle of 78° with respect to the surface normal. Also, all spectra were obtained at 4 cm⁻¹ resolution for 1000 scans. Minimal baseline correction was applied to all spectra.

X-ray Photoelectron Spectroscopy (XPS). XPS spectra were obtained with a PHI 5700 ESCA X-ray photoelectron spectrometer using Mg Kα radiation (15 kV, 225 W, base pressure 5-10⁻¹⁰ Torr). Survey spectra were collected at constant pass energy of 160 eV from a 0.37×1.0 mm² area of the sample. High-resolution spectra of all the detected elements were collected at pass energy of 9 eV. The binding energies was corrected by referencing the C(1s) binding energy to 285 eV.

Preparation of functional SAMs on 316L SS. 316L SS sample plates (20 mm×20 mm×2 mm) were polished by using a Handimet Grinder polishing machine with 4 roughness of polishing papers (240, 320, 400, and 600 grit papers). The roughness of the polished 316L SS plates was measured by a profilometer as 0.2±0.1 μm. The samples were cleaned chemically as follows: ultrasonicated for 10 minutes each in 70 percent ethanol, acetone and 40 percent nitric acid. This treatment is hereafter referred to as the “chemical treatment.”

Then the SS plates were subjected to glow discharge gas plasma (GDGP) treatment in a radio frequency glow discharge system (Harrick Scientific, NJ) for 3 minutes in an oxygen environment under reduced pressure (15 psi). The plates were then immediately dipped in amphiphile solutions for 48 hr of either 11-mercapto-1-undecanol (—OH SAM) or 16-mercaptohexadecanoic acid (—COOH SAM) to form respective functional SAMs on 316L SS.

Preparation of Amphiphile Solutions of 16-Mercaptohexadecanoic Acid: 16-Mercaptohexadecanoic acid (HSCH₂(CH₂)₁₃CH₂COOH) was dissolved in ethanol/water/acetic acid (85/10/5, v/v/v) to form a 2 mM solution.

Preparation of Amphiphile Solutions of 11-Mercaptol-undecanol: 11-Mercaptol-undecanol [HOCH₂(CH₂)₉CH₂SH] was dissolved in ethanol to form a 10 mM solution. After 48 hrs of immersion in amphiphile solutions, the SS samples were rinsed and washed with ethanol and ultrapure water, and then characterized using XPS, FTIR and contact angle measurements.

Lipase catalyzed esterification of therapeutic drugs on SAMs. Functional SAMs on 316L SS were used as precursors to attach therapeutic moieties on steel as follows: samples of the functional SAMs on 316L SS were put in a 100 ml beaker containing 10 ml toluene to which 50 mg of the drug (perphenazine for —COOH terminated SAMs and ibuprofen for —OH terminated SAMs) was added. Finally, 10 mg of Novozyme-435 was added as a biocatalyst. The beaker was covered with aluminum foil and was placed in a shaking water bath maintained at 60° C. for 5 hr. After 5 hr, the steel plates were removed, washed and rinsed with ethanol, acetone and double-distilled (dd) water. These samples were then characterized using XPS, FTIR and contact angle measurements.

### Results and Discussion

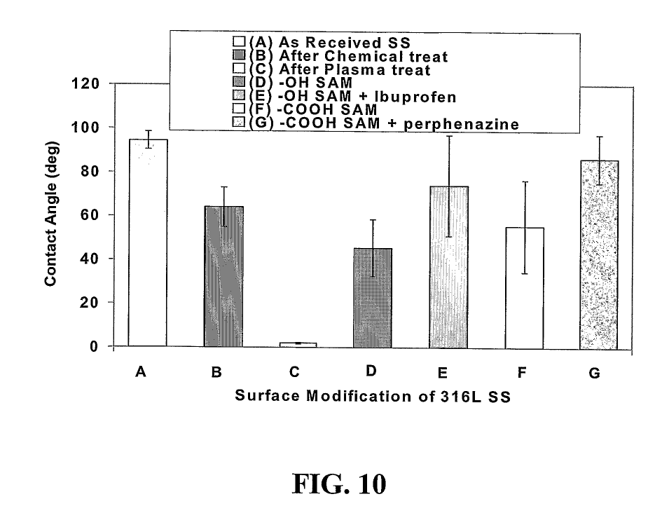

Preparation and characterization of functional SAMs on 316L SS. Gas plasma treatment (3 min) was used in a reduced pressure oxygen environment to improve surface hydrophilicity as evident by contact angle of less than 3° after gas plasma treatment as compared to 64°±8° after chemical cleaning only (FIG. 10). After chemical and gas plasma treatment, the 316L SS plates were immediately dipped in amphiphile solutions of 16-mercaptohexadecanoic acid and 11-mercapto-1-undecanol for 48 hr to form —COOH and —OH functional SAMs, respectively, on 316 L SS. The SAMs formed were characterized using XPS, FTIR, and contact angle measurements

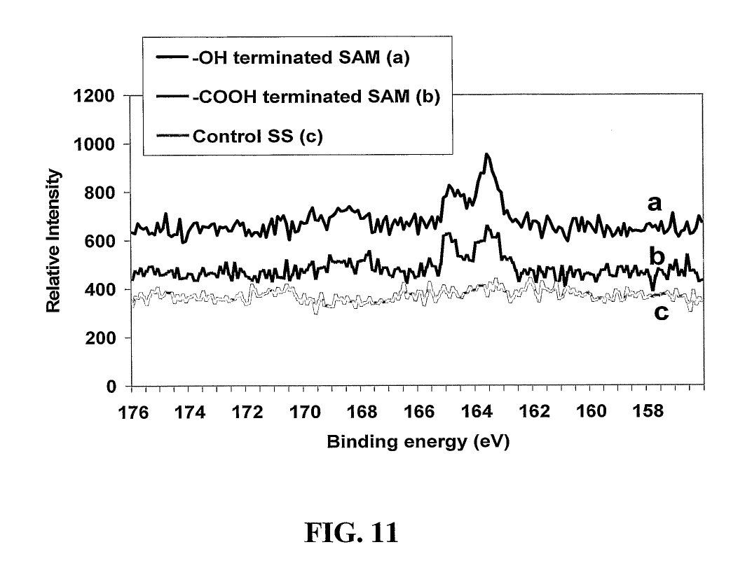

FIG. 11 shows the S (2p_{3/2}) region for the —OH SAMs and —COOH SAMs. The XPS spectrum shows a peak at 163 eV representing the metal thiolate. This binding energy of the S (2p_{3/2}) peak for the thiol monolayer falls within the range (160-165 eV) (Flynn et al., 2003) and is consistent for thiol SAMs adsorbed on copper, silver, gold and iron (Laibinis et al, 1991). The identity of adsorbate as the metal thiolate rather than as an oxidized sulphur is clear due to lack of peaks for sulfinates and sulphonates (binding energy 168 eV) (Ruan et al, 2002; Schoenfisch and Pemberton, 1998).

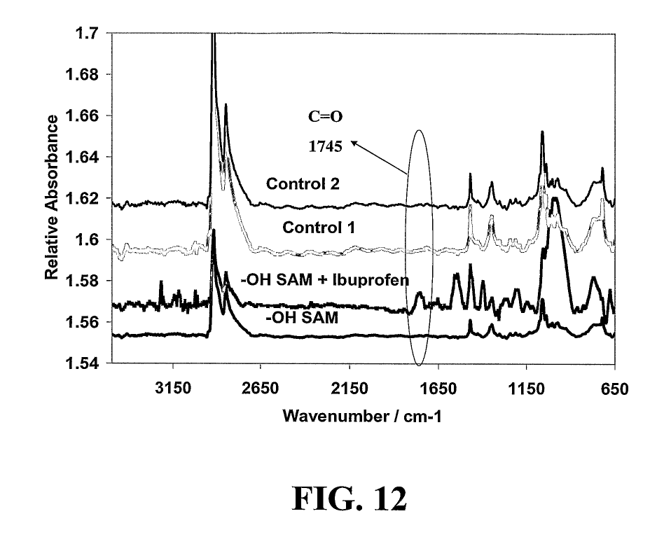

To further verify formation of thiol SAMs on 316L SS, the surfaces were characterized by FTIR spectroscopy. The methylene stretching of the alkyl chains for both —COOH and —OH terminal SAMs are clearly visible with two absorption bands at 2917 and 2846 cm⁻¹ (FIG. 12). These bands have been previously assigned to CH₂ asymmetric and CH₂ symmetric vibrations, respectively, for other alkanethiol SAMs on gold (Flynn et al., 2003). The spectrum of the —COOH SAM (FIG. 12) shows the C═O stretching band at 1706 cm⁻¹ in the mid IR spectrum suggesting that most of the terminal acid groups are participating in intermolecular hydrogen bonding processes (Duevel and Corn, 1992; Yan et al., 1997). The —OH SAM spectrum shows the C—O stretch absorption at 1040 cm⁻¹, thus validating the formation of SAMs on 316L SS.

Contact angle measurements (FIG. 10) show significant differences in contact angle between bare SS after GDGP (less than 3 deg) and those of the functional SAM formed (—OH SAM: 45°±13°, —COOH SAM: 55°±21°). Ruan et al., 2002, have previously reported that the water contact angle of 11-mercapto-1-undecanol is not stable under ambient conditions, likely due to result of surface reorganization. A similar phenomenon has also been observed for such monolayer on a gold surface (Laibinis and Whitesides, 1992), which could possibly explain the high standard deviation values observed in our case.

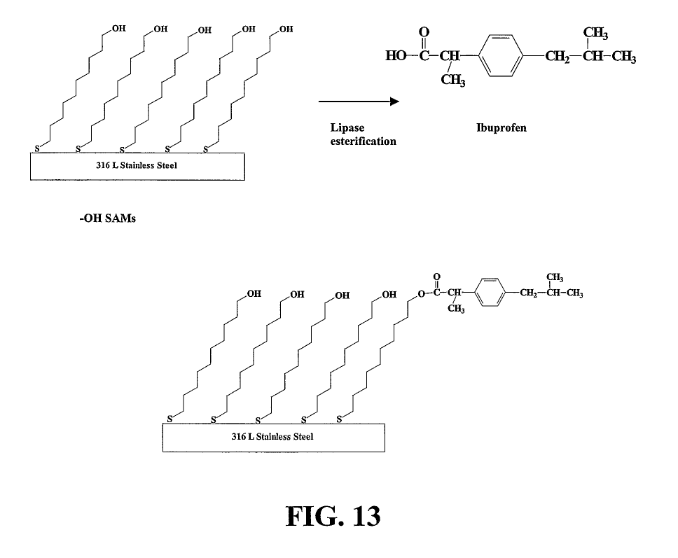

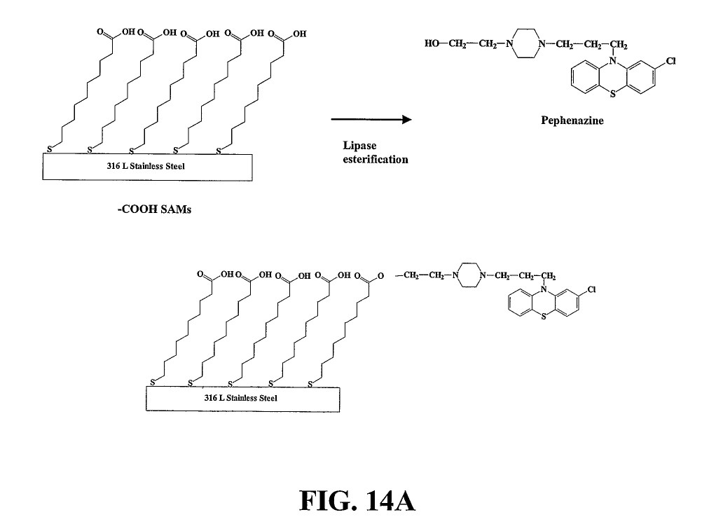

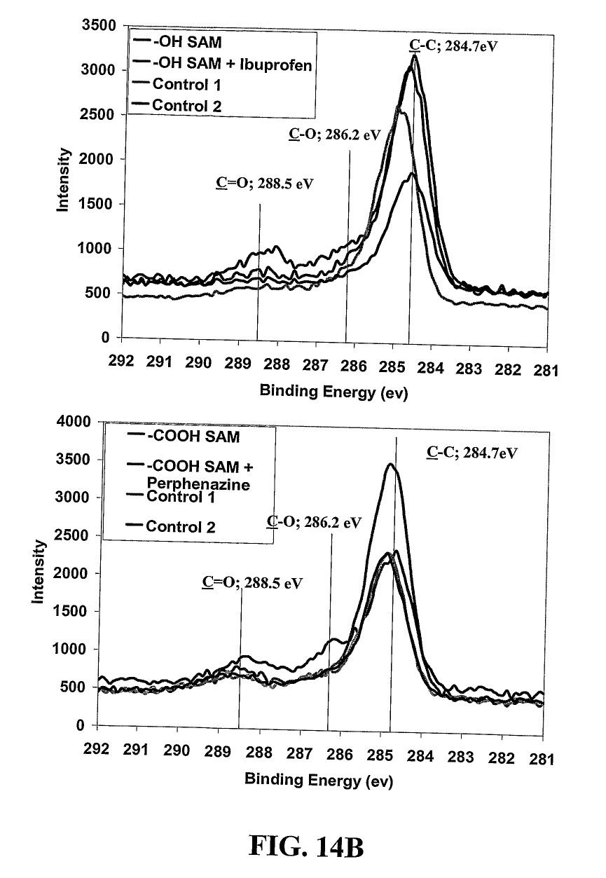

Surface modification of functional SAMs via lipase catalysis. Functional SAMs on 316L SS, prepared using 1-mercapto-1-undecanol (—OH SAM) or 16-mercaptohexadecanoic acid (—COOH SAM) were used as precursors to attach therapeutic moieties for cardiovascular implant applications. Drugs were selected for lipase catalyzed surface modification because they had the appropriate functionalities; ibuprofen has a COOH functional group that could be attached to the —OH SAM (FIG. 13), whereas perphenazine has a OH functional group that could be attached to the —COOH SAM (FIG. 14A).

These drugs were selected to demonstrate proof of concept, however similar reactions could be easily extended to other esterification reactions on SAMs. The reaction was carried out in toluene at 60° C. for 5 hrs using Novozyme-435 as the biocatalyst. Selection of Novozyme-435 was based on previous reports of it being the preferred biocatalyst for esterifications reactions (Mahapatro et al., 2004; Mahapatro et al., 2003). Toluene was selected as the preferred solvent based on previous studies of lipase catalyzed esterifications in various process conditions (Mahapatro et al., 2003).

The drug attachment to the functional SAM was characterized via FTIR (FIG. 12). After the surface modification of —OH SAMs via lipase catalysis, the presence of the C═O stretching bands at 1745 cm⁻¹ can be seen, which was absent in the FTIR spectra of —OH SAMs. The C═O band should only evolve after the esterification of the carboxylic moiety of the drug (ibuprofen) with the OH terminal SAM via enzyme catalysis. Similarly, for the FTIR spectra before and after the reaction of the —COOH SAM (FIG. 12) with perphenazine we see that before surface modification there is a C═O peak at 1706 cm⁻¹ which suggests that the terminal acid groups are participating in intermolecular hydrogen bonding processes (Duevel and Corn, 1992; Ruan et al., 2002).

After esterification, two peaks are seen in the carbonyl region, a peak at 1764 cm⁻¹, which is the representative peak for the C═O stretching for esters. The second peak at 1681 cm⁻¹ is the C═O stretching of the remaining unreacted terminal COOH; the reduction in the stretching frequency suggests stronger intermolecular bonding of the unreacted terminal groups. This suggests that esterification reaction has occurred giving different peaks for carbonyl of carboxylic acids and ester respectively. Control reactions (a) with drug and without Novozyme-435 (Control 1) and (b) with Novozyme-35, but without the drug (Control 2) were carried out to confirm that these reactions occur via lipase catalysis and to see the possibility of any non specific adsorption of the lipase to the metal surface (FIG. 12). The spectra obtained were similar to that of the functional SAM only which proves that the reaction has taken place due to lipase catalysis. This also suggests non existence or negligible non specific adsorption of the lipase to the metal surface. Previous work on evaluating catalytic activity versus time in polyesterification reactions showed no significant loss in activity (<7%) of Novozyme-435 after 4 hr for solvent less polycondensations of hydroxyl acids carried out at 90° C. (Mahapatro et al., 2004) and significant higher retained enzymatic activity in organic solvent (diphenyl ether) as compared to bulk (solvent less systems) for polyesterification reactions of diacids with diols.

This suggests that for the process condition used (60° C., 5 hr, in toluene), the lipase does not leach out from the immobilized beads which could lead to nonspecific adsorption of lipase to the metal surface, as may be the case in aqueous protein systems interactions with solid surfaces (Norde, 1986).

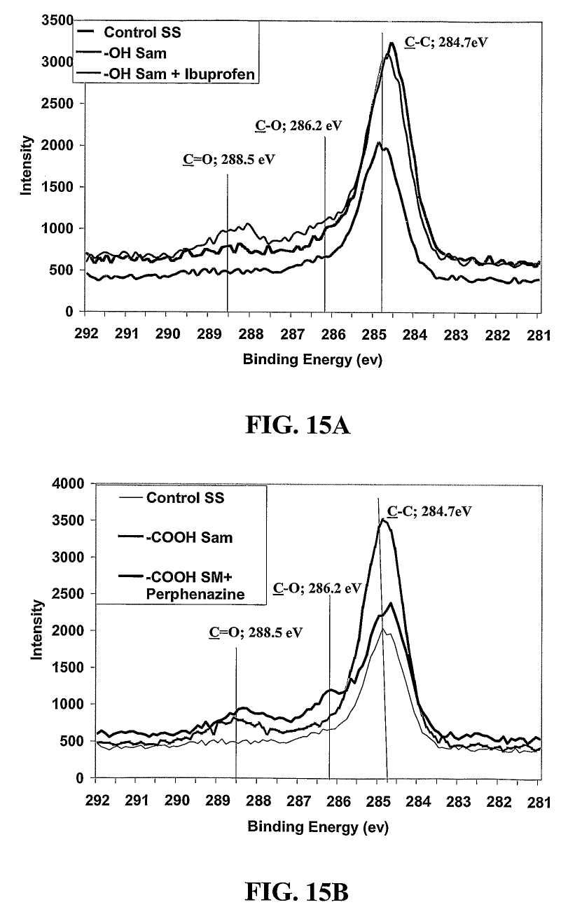

XPS analysis was carried out to further characterize drug attachment. FIG. 15 shows the C (1s) region for the —OH SAM before and after surface modification. The spectrum of the hydroxyl thiol SAM exhibits a slightly asymmetric photoelectron peak centered at 284.7 eV, which is characteristic of the carbon ‘C’ in the internal units of the methylene chain (CH₂CH₂CH₂) (Palegrosdemange et al., 1991; Bain et al., 1989). After lipase catalysis with ibuprofen, a photoelectron peak was seen evolving at 288.5 eV which arises from the ‘C’ (C═O) of the carboxylic acid (Bain et al., 1989). This would evolve only after the esterification of ibuprofen with the —OH SAM. Similarly, the spectrum of the carboxyl thiol SAMs exhibits a slightly asymmetric photoelectron peak centered at 284.7 eV, which is characteristic of the internal units of the methylene chain (CH₂CH₂CH₂). A photoelectron peak evolving at 288.5 eV was also seen, which arises from the “C” (C═O) of the carboxylic acid.

After lipase catalysis with perphenazine, a small photoelectron peak evolving at 286.5 eV can also be seen, which corresponds to the ‘C’ in the methylene groups adjacent to the oxygen (C—O) (Palegrosdemange et al., 1991). This should evolve only after the esterification of perphenazine with the —COOH SAM. Control reactions (a) with drug and without Novozyme-435 (Control 1) and (b) with Novozyme-435, but without the drug (Control 2) were carried out to confirm that these reactions occur via lipase catalysis and to estimate the likelihood of any non specific adsorption of the lipase to the metal surface (FIG. 14B. The spectra obtained were similar to that of the functional SAM only, which strongly suggests that the reaction has taken place due to lipase catalysis. This also suggests nonexistence or negligible nonspecific adsorption of the lipase to the SAMs as discussed previously.

Contact angle measurement data (FIG. 10), after drug attachment shows increase in contact angle after drug attachment (45.40±12.8° for —OH SAM to 74°±22.6° after drug attachment and 55.4°±21.0° for —COOH SAM to 86°±10.6° after drug attachment). Statistical analysis using one way ANOVA was performed and it was determined that these increases were significant at p<0.05. The high standard deviation could possibly be due to surface reorganization of SAM.

These results demonstrate lipase catalyzed esterification of therapeutic drugs to functional SAMS. The potential steric bulk of the surface in addition to the highly ordered nanostructure of the SAMs does not appear to affect the catalytic activity of the lipase.

### Example 4

### Formation of SAMs on Gold and Titanium Surfaces

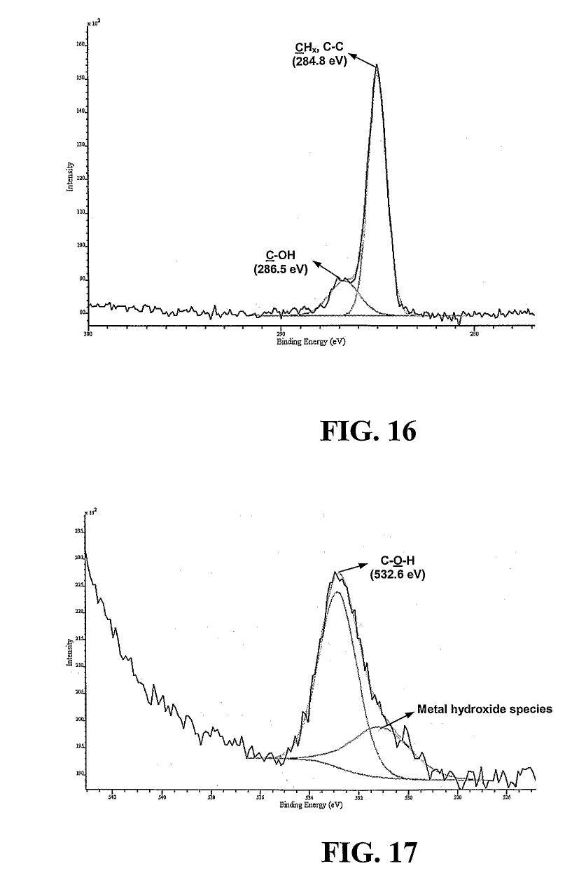

FIG. 16 and FIG. 17 shows the high resolution XPS spectra of the C 1s and O 1s region for the —OH terminated SAMs on gold substrates. The high resolution C 1s spectrum is deconvoluted into two components: the BE of 284.8 eV may be attributed to C—C and CHₓ species (Ren et al., 2003), while 286.5 eV may be attributed to the terminal carbon atom which is attached to the —OH species of the monolayers formed (Pan et al., 1998; Hutt and Leggett, 1997). Also, the large peak at 532.6 eV in the O 1s spectrum is assigned to the oxygen atoms in the terminal hydroxyl group (Abdureyim et al., 2001; Rjeb et al., 2004). The peak at 531.2 eV in the O 1s spectrum would have arisen because of the metal hydroxide species (Alexandrescuyz et al., 1997; Knotek, 1998). The large and prominent —OH components observed in both the C 1s and O 1s spectra may indicate the uniformity of SAMs. This confirms the formation of orderly SAMs on the gold substrates.

Commercially pure titanium (cp-Ti) plates of thickness 0.062 inches were used in the experiments. The roughness of as-received (control sample) cp-Ti plates were measured as 0.7±0.1 μm by using the profilometer. Then the plates were polished, and the roughness was calculated as 0.3±0.1 μm. The samples were manually polished by using the Handimet Grinder polishing machine with 4 types of grit papers (240, 320, 400, and 600 grit papers). The polished titanium plates were then chemically cleaned. The samples were cleaned with 70% ethanol, acetone and 40% nitric acid in ultrasonication for 10 minutes and then air dried.

The samples were treated with ethanol for removing oils and greases, and with acetone for drying the samples, and finally with nitric acid for passivating the sample surfaces. After the chemical cleaning, the samples were oxygen gas-plasma treated at high intensity for 3 minutes. As soon as the samples were plasma treated, they were dipped in the amphiphile solutions of phosphate, phosphonic acid, and trichloro silane SAMs for 48 hours. After that, the samples were taken out and rinsed with water. After each stage of treatment described above, contact angle measurements were made on the Ti samples (FIG. 18). The contact angle decreased significantly (p<0.01) after glass plasma treatment indicating a hydrophilic surface. Following methyl-terminated phosphate, phosphonic acid, and trichlorosilane SAMs formation, the angle increased to >100°, which is typical for well defined alkyl phosphate SAMs (Hofer et al., 2001), confirming the formation of SAMs on the titanium surfaces.

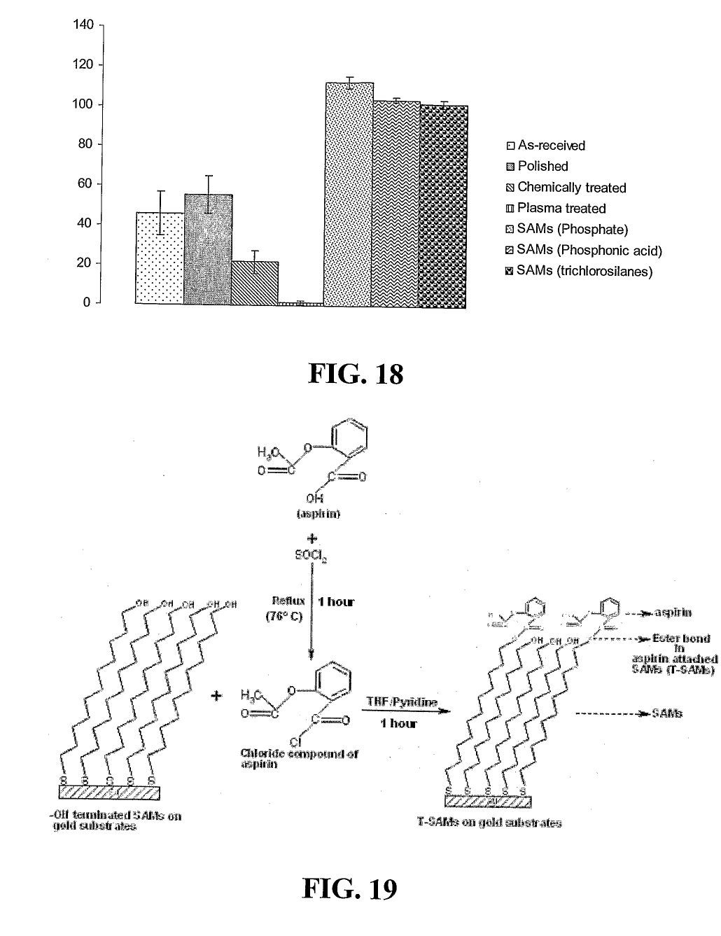

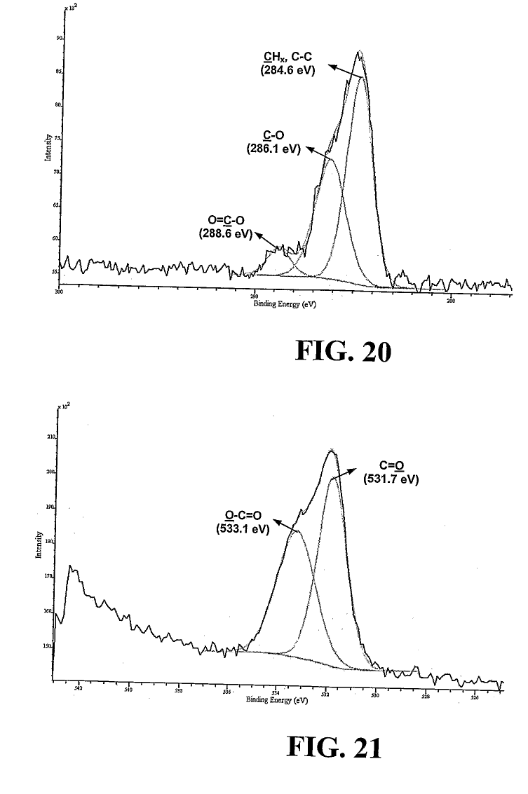

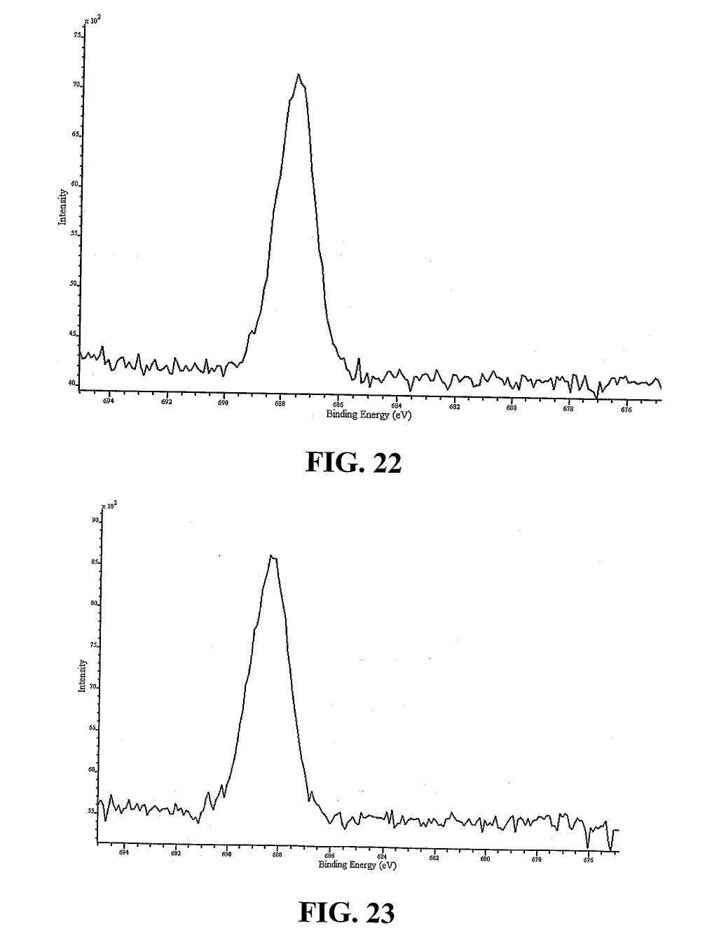

Attachment of therapeutics to the SAMs on gold surfaces. The T-SAMs formed by chemically attaching aspirin to the SAMs as represented in the schematic diagram (FIG. 19), were also characterized by XPS (FIG. 20 and FIG. 21). The higher BE in the C 1s spectrum of T-SAMs_{(Asprin)} formed specimens at 286.1 eV and 288.6 eV is assigned to the newly formed ether (C—O—C) (Yoshida et al., 2004) and ester (O═C—O) (Yoshida et al, 2004) bonds after the attachment of aspirin.

Also, the formation of these bonds is further supported by the higher BE peaks in the O 1s spectrum; the peaks at 531.7 eV and 533.1 eV are assigned to carbonyl oxygen (Abdureyim et al., 2001; Rjeb et al., 2004) and ester bonds (Onyiriuka, 2003), respectively (FIG. 21). To prove the concept, two other drug molecules, diflunisal and flufenamic acid, were chosen for the presence of unique elements like fluorine and nitrogen. The detection of these elements using XPS guarantees the presence of the drug because these elements are neither present in the SAMs nor on the gold substrates. In addition, as there is no ester bond present in either of these drug molecules outside of the point of attachment, the presence of an ester bond peak in the XPS confirms the attachment of the drug to the SAMs.

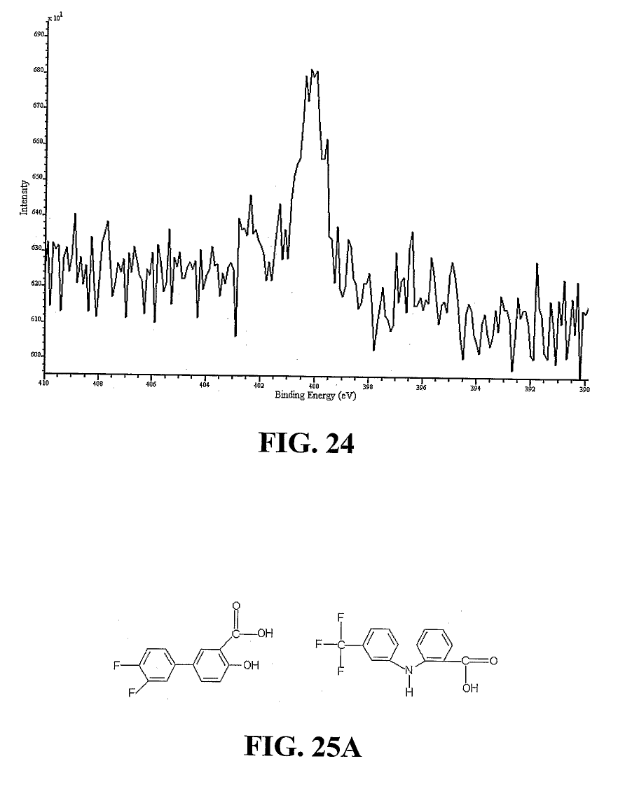

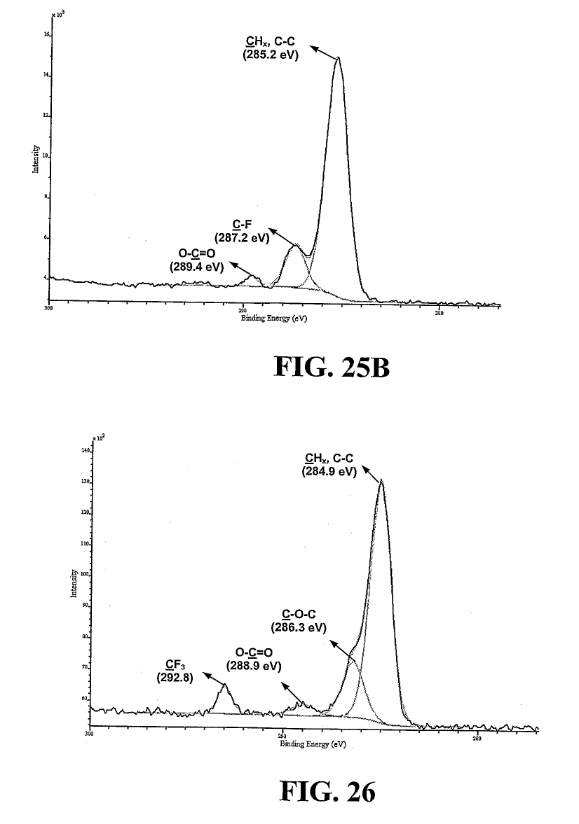

The large and symmetrical F 1s and N 1s peaks (FIGS. 23, 24, and 25) observed on the drug attached samples strongly confirmed the presence of diflunisal and flufenamic acid on the SAMs formed substrates. The C 1s peaks at 287.2 eV and 292.8 eV are assigned to the formation of C—F (Ada et al., 1998; Wagner et al., 2000) and —CF₃ bonds (Wagner et al., 2000; Wistara et al., 1999) in diflunisal and flufenamic acid attached samples respectively (FIG. 25 and FIG. 26). These unique peaks arise only after the attachment of the drugs to the SAMs.

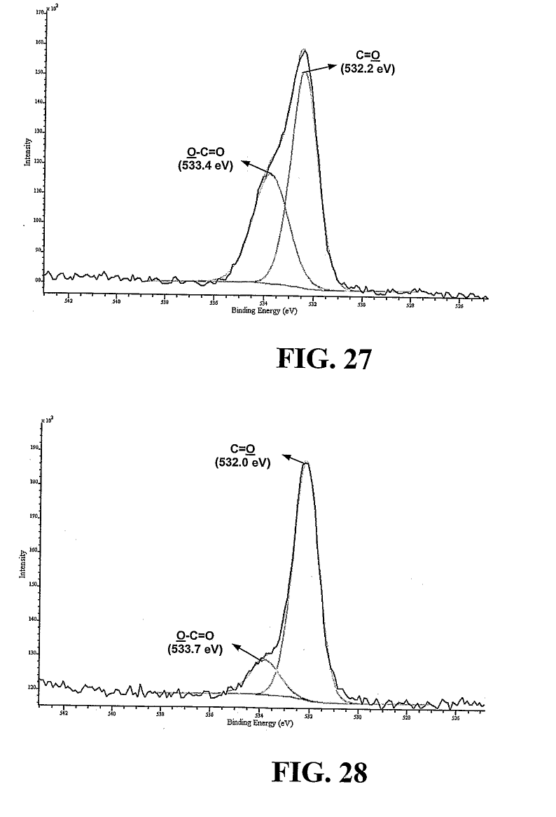

The formation of ester bonds between the SAMs and the drug molecules is confirmed by the higher BE peaks at 289.4 eV and 288.9 eV of the C 1s spectrum (FIG. 25 and FIG. 26) for the T-SAMs_{(Diflunisal)} and T-SAMs_{(Flufenamic acid)} formed specimens respectively (Gea and Turunen, 2003; Cumpston et al., 1997). The ester bond formation is further confirmed with the higher BE peaks at 533.4 and 533.7 eV in O 1s spectrum (FIG. 27 and FIG. 28) of T-SAMs_{(Diflunisal)} and T-SAMs_{(Flufenamic acid)} formed specimens respectively (Konstadinidis et al., 1992; López et al., 2004).

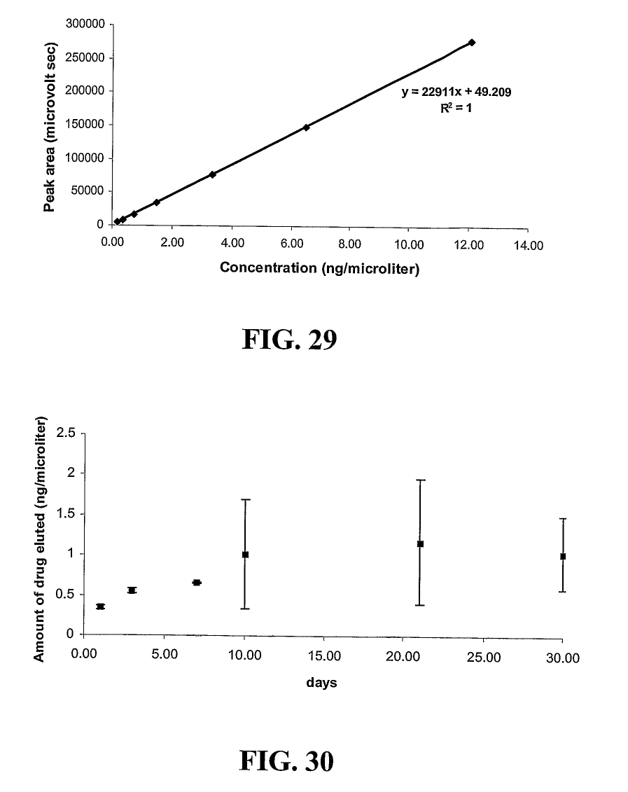

Drug elution studies on T-SAMs coated gold substrates. Stock solution containing 10 mg/10 ml aspirin in mobile phase was used. The stock solution was then diluted in the mobile phase to furnish solutions with concentrations of 0.19, 0.39, 0.74, 1.50, 3.35, 6.49, 12.09 ng/l. Calibration curves were obtained by plotting peak area ratios versus concentration of aspirin (FIG. 29). Aspirin showed linearity in the range of 0.2-12.09 ng/μl. The slope, intercept, and correlation coefficient values were found to be 22911 microvolt sec/(ng/microliter), 49.209 microvolt sec, and 1 respectively.

Subsequently, the drug release study of the T-SAM coated gold samples was carried out over a period of 30 days using HPLC. Six different samples were used at each time points. All the six samples were characterized at 1, 3, 7, 10, 21, and 30 days. The characterization data is presented in FIG. 30. It was observed that there was an increase in drug elution during the first week of trials. The quantity of drug released was found to plateau after about 10 days, though a large variance was found for the data sets collected day 10 onwards.

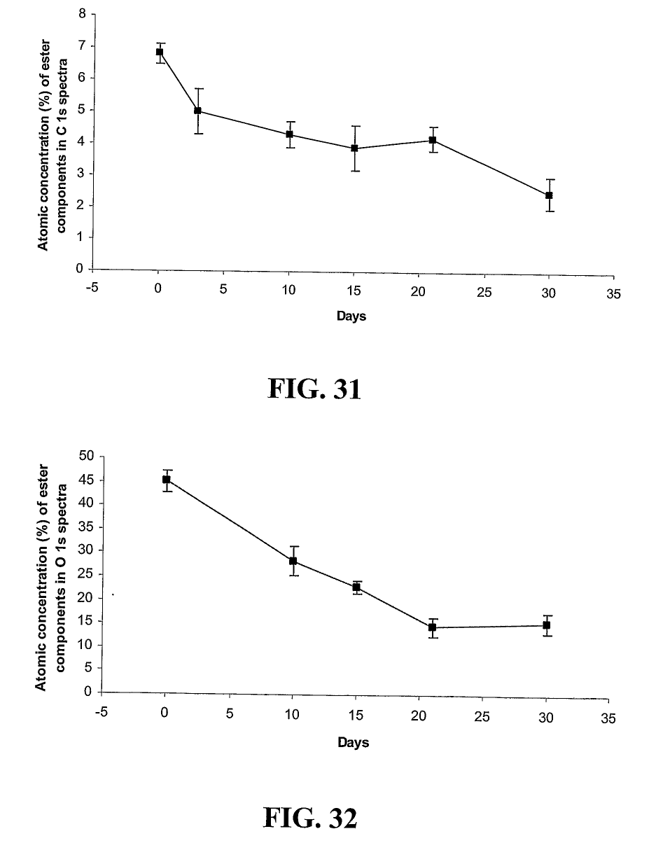

XPS measurements were taken for the aspirin gold substrates. The C 1s, O 1s, and S 2p spectra were thoroughly investigated for determining elemental composition changes on the samples after drug elution. In the C 1s spectrum, the mole fraction of the ester peak at 288.6±0.2 eV was determined with respect to the other two peaks; hydrocarbons at 284.6±0.1 eV and ether at 285.9±0.2 eV; and plotted in FIG. 31.

Similarly, in the O 1s spectrum, the mole fraction of the ester peak at 533.2±0.2 eV was determined with respect to the carbonyl peak at 531.9±0.1 eV, and plotted. The relative peak widths of the mole fractions of ester, ether, and hydrocarbon peak were kept constant. FIG. 31 and FIG. 32 show the drug-SAM ester bond hydrolysis profile. This demonstrates that the drug release is occurring in a controlled fashion via a hydrolytic mechanism involving cleavage of the drug-SAM ester bond.

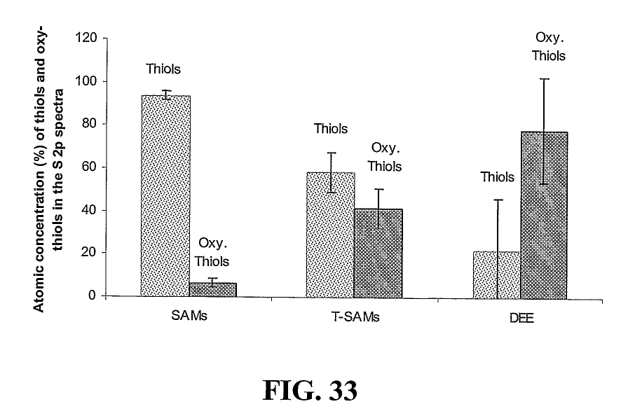

Investigating the stability of SAMs on gold and titanium surfaces. It is important to note that approximately 40% of the thiol species in T-SAMs were oxidized during the drug attaching experiments (FIG. 33). It is ideally desired to keep the oxidation % of thiol species to a minimum during the drug loading procedure so as to ensure greater stability of SAMs which would result in lesser variation in long term drug elution.

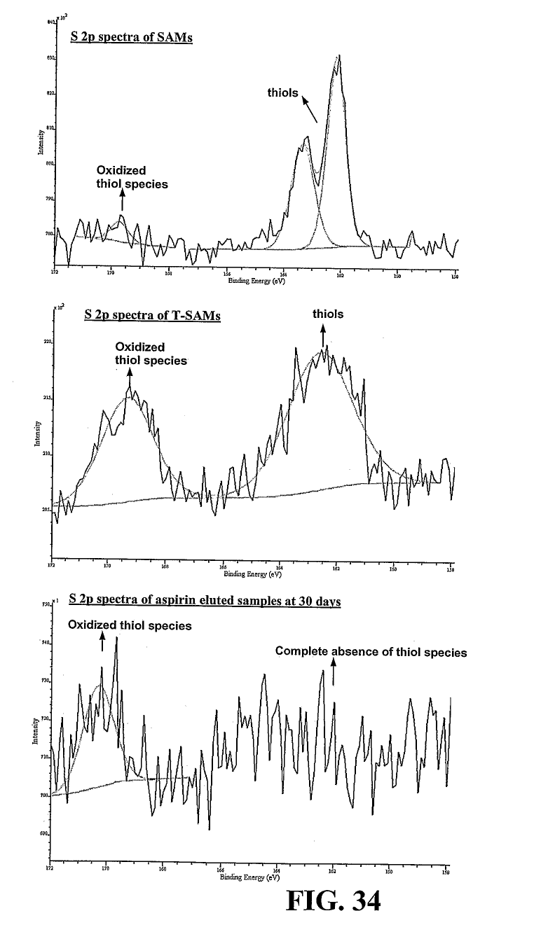

Drug delivery through T-SAMs may be limited by stability of self assembled monolayers (Lee et al., 2004; Ishida et al., 2002; Lee et al., 1998; Flynn et al., 2003). FIG. 34 shows the XPS spectra of the S 2p region after the formation of SAMs, T-SAMs, and after the elution of the drugs. The S 2p spectra of all the samples have been examined carefully for the peaks at 162 eV (for thiol species) and 169 eV (for oxidized thiol species-sulfonates) (Lee et al., 2004; Ishida et al., 2002; Lee et al., 1998). FIG. 33 shows the atomic concentration (%) of the thiol (BE=162.2±0.4 eV) components and oxidized thiol (BE=169.3±0.6 eV) components in the XPS S 2p spectra.

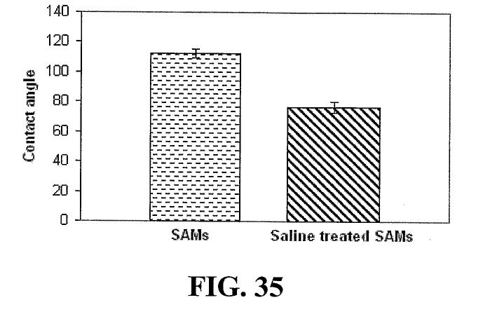

The stability of SAMs on titanium was checked by dipping the phosphate SAMs formed Ti plates in the 0.09% saline solution for 48 hours. The measured contact angle decreased from 112.4±3° to 76.8±3.7°. The stability of the SAMs was examined by immersing the phosphate SAMs formed metal plates in 0.09% saline solution for 48 hours. The contact angle was reduced from 112.4±3° to 76.8±3.7° (FIG. 35). This shows the disorderly arrangement of SAMs after the saline solution treatment. According to one way ANOVA, since the calculated F ratio (921.83) is markedly greater than any of the critical values (13.12) in the table, the saline solution has a very significant effect on the stability of SAMs at p<0.001. The power is >0.995, the number of samples used=22.

### Example 5

### Protocols to Investigate the Stability of Phosphates Phosphonic Acids and Trichlorosilane SAMs on Titanium Surfaces and to Compare it with the Stability of SAMs on Standard Gold/Thiol Systems

Formation of SAMs on gold. Glass microscope slides used for gold substrates will be first cleaned with detergent solution, acetone, and an ample amount of double-distilled water (dd-H₂O) for 30 minutes. The cleaned glass slides will be sputter coated with a 20 nm layer of titanium to improve adhesion of gold on the glass substrates. This will be followed by 150 nm thick gold deposition at a growth rate of 1-2 nm/s. The gold substrates will be rinsed with absolute ethanol and dd-H₂O repeatedly for 3 minutes before the SAMs deposition. The 2 mM solutions of 11-mercapto-1-undecanol will be prepared with absolute ethanol. The SAMs will be formed by immersing gold substrates into the above prepared solutions for 48 hours. Upon removal, the samples will be rinsed with ethanol for 3 minutes, and blown dry with nitrogen.

Formation of SAMs on titanium. Titanium substrates will be prepared by sputter coating the cleaned glass slides with a 400 Å layer thick titanium deposition. Phosphate SAMs will be formed on the titanium substrates by immersing them for 48 hours in the amphiphile solution of ammonium salt of dodecyl phosphate, dissolved in water. The synthesis of ammonium salt of dodecyl phosphate and the preparation of its amphiphile solution will be carried out as per previously reported synthetic protocols for these monolayers. Phosphonic acid SAMs will be formed on the titanium substrates by immersing them for 48 hr in the amphiphile solution of carboxyl alkyl-phosphonic acid, which consists of 2 mM solution of the carboxyl alkyl-phosphonic acid, dissolved in ethanol. Synthesis of carboxyl alkylphosphonic acid will be carried our as per reported literature protocol (Pawsey et al., 2002). SAMs will be formed on the titanium substrates by immersing them for 48 hours in the amphiphile solution of trichlorosilanes, dissolved in toluene.

Investigating the stability of SAMs. The phosphate, phosphonic acid, and trichlorosilane SAMs formed will be evaluated for their stability in air, PBS and UV light. For measuring the stability in ambient laboratory conditions and UV light, SAMs will be exposed to normal atmospheric air and UV light at various time intervals (1, 3, 7, 10, and 15 days) respectively. After respective time intervals, the samples will be rinsed in ethanol and dd-H₂O to remove the physio-adsorbed molecules. Then, the samples will be analyzed using contact angle measurements and XPS. At least 6 samples will be used for each time point. SAMs stability in PBS will be determined in a similar manner by immersing the SAMs in PBS for similar time intervals (1, 3, 7, 10, and 15 days). Samples will be immersed in 10 ml of PBS (pH 7.4) at 37° C. After respective time intervals the samples will be rinsed in ethanol, and dd-H₂O. The samples will then be analyzed for stability using contact angle measurements and XPS, as described above for air stability of SAMs. Similar experiments on stability will be carried out on gold/thiol systems. The data on phosphate, phosphonic acid, trichlorosilane SAM's stability on titanium will be compared to the stability data on gold/thiol systems.

### Example 6

### Additional Protocols to Attach Therapeutic Molecules to Gold and Titanium Surfaces Via SAMs

Attachment of therapeutics to the SAMs on gold substrates. Before functionalizing the SAMs on gold substrates, 0.25 grams of aspirin will be refluxed in 4 ml of thionyl chloride at 76° C. for 1 hour in nitrogen atmosphere. The excess of thionyl chloride will be removed by evaporation under vacuum on a rotary evaporator at 40° C. 20 ml of dry-tetrahydrofuran (THF) will be injected into the prepared acid chloride. Hydroxyl terminated SAMs formed gold substrates will then carefully immersed into the solution mixture. Then 0.2 ml of pyridine will be added, and the mixture will be kept under nitrogen purge for one hour. The substrates will be rinsed with THF for 3 minutes and blow dry with nitrogen.

Attachment of therapeutics to the SAMs on titanium substrates. A solution mixture of 0.25 grams of diflunisal, 20 ml of THF, and 0.2 ml of pyridine will be prepared. The carboxy-alkyl phosphonic acid SAMs formed substrates will be immersed in thionyl chloride for 20 minutes in the nitrogen atmosphere. After 20 minutes, the substrates were taken out and transferred immediately to the prepared solution mixture and it will be kept under nitrogen purge for one hour. Then, the substrates will be rinsed with THF for 3 minutes and blow dry with nitrogen.

### Example 7

### Protocol to Assess the Drug Release Kinetics of T-SAMs Coated Gold and Titanium Surfaces

Cumulative drug release study on T-SAMs formed gold and titanium substrates. T-SAMs formed gold and titanium substrates will be submerged in 7 ml of phosphate buffered saline solution (PBS, pH 7.4) at 37° C. A PBS sample will be taken at 1, 3, 7, 10, 21 and 30 days and analyzed for the quantity of drug eluted. The gold and titanium substrates with SAMs, T-SAMs, and post drug elution will be characterized by X-ray photoelectron spectroscopy. XPS data will be collected on three points on each specimen in order to ensure that local inhomogenities do not affect the results. The PBS solution with eluted drug will be characterized by high performance liquid chromatography. The amount of drug that is coated on the sample surface will be determined by using AFM/STM molecular imaging techniques.

All of the devices and methods disclosed and claimed herein can be made and executed without undue experimentation in light of the present disclosure. While the compositions and methods of this invention have been described in terms of preferred embodiments, it will be apparent to those of skill in the art that variations may be applied to the devices and methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,680,338
U.S. Pat. No. 5,092,877
U.S. Pat. No. 5,141,648
U.S. Pat. No. 5,304,121
U.S. Pat. No. 5,563,250
U.S. Pat. No. 5,609,907
U.S. Pat. No. 5,721,131
U.S. Pat. No. 5,852,127
U.S. Pat. No. 5,856,456
U.S. Pat. No. 5,879,697
U.S. Pat. No. 5,880,270
U.S. Pat. No. 5,897,588
U.S. Pat. No. 5,938,695
U.S. Pat. No. 6,017,365
U.S. Pat. No. 6,025,202
U.S. Pat. No. 6,053,942
U.S. Pat. No. 6,068,656
U.S. Pat. No. 6,099,562
U.S. Pat. No. 6,146,767
U.S. Pat. No. 6,242,264
U.S. Pat. No. 6,287,332
U.S. Pat. No. 6,398,804
U.S. Pat. No. 6,440,565
U.S. Pat. No. 6,532,380
U.S. Pat. No. 6,562,066
U.S. Pat. No. 6,617,027
U.S. Pat. No. 6,617,527
U.S. Pat. No. 6,723,517
U.S. Pat. No. 6,756,354
U.S. Pat. No. 6,821,529
U.S. Pat. No. 6,875,227
U.S. Pat. No. 6,893,413
U.S. Patent Appln. Pub. 20020119305
U.S. Patent Appln. Pub. 20020164419
U.S. Patent Appln. Pub. 20020197879
U.S. Patent Appln. Pub. 20030059865
U.S. Patent Appln. Pub. 20030114921
U.S. Patent Appln. Pub. 20030125798
U.S. Patent Appln. Pub. 20030157732
U.S. Patent Appln. Pub. 20030158460
U.S. Patent Appln. Pub. 20040062592
U.S. Patent Appln. Pub. 20040037836
U.S. Patent Appln. Pub. 20040116999
U.S. Patent Appln. Pub. 20050060022
U.S. Patent Appln. Pub. 20050084515
U.S. Patent Appln. Pub. 20050085895
U.S. Patent Appln. Pub. 20050085898
U.S. Patent Appln. Pub. 30020130719
U.S. Patent Appln. Pub. 20030130611
Abdureyim et al., J. Electron Spectro. Rel. Phenom., 114-116:371-374, 2001.
Abizaid et al., Circulation, 104(5):533-538, 2001.
Ada et al., J. Phys. Chem. B, 102:3959-3966, 1998.
Al Suwaidi et al., Am. J. Cardiol., 87(6):680-686, 2001.
Alexandrescuyz et al., J. Phys. Appl. Phys., 30:2620-2625, 1997.
Allara et al., J. Amer. Chem. Soc., 113:1852-1854, 1991.
Amir et al., Angew. Chem. Int. Ed., 42:4494-4499, 2003.
Anderson and Hendifar, J. Amer. Chem. Soc., 81:1027-102, 1959.
Bain et al., J. Am. Chem. Soc., 111:321-335, 1989.
Biltresse et al., J. Biomaterials, 26:(22):4576-4587, 2005.
Bonan et al., Am Heart J., 121(5):1522-1530, 1991.
Castner and Ratner, Science, 500(1-3):28-60, 2002.
Chapman et al., Langmir, 16(17):6927-6936, 2000.
Chau and Porter, Chemical Physics Letters, 167(3):198-204, 1990.
Chen et al., Materials Research Bulletin, 36(15):2605-2612, 2001.
Colombo et al., Circulation, 108(7):788-794, 2003.
Cumpston et al., J. Appl. Phys., 81(8):3716-3720, 1997.
Cura et al., Am J Cardiol. Mar. 1, 2003; 91(5):587-590, 2003.
Duevel and Corn, Anal. Chem., 64:337-342, 1992.
Ellis, In: Angiographic results from the pivotal, prospective, randomized trial of the slow-rate release of polymer-based paclitaxel-eluting TAXUS stent, Transcatheter Cardiovaculsr Therapeutics (TCT) Meeting, Washington, D.C., 2003.
Fadeev and McCarthy, J. Amer. Chem. Soc., 121:12184-12185, 1999.
Farb et al., Circulation, 104(4):473-479, 2001.
Fischman et al., N. Engl. J. Med., 331(8):496-501, 1994.
Flynn et al., Langmuir, 19:10909-10915, 2003.
Flynn et al., Langmuir, 19:10909-10915, 2003.
Folkers et al., Langmuir, 11(3):813-824, 1995.
Frey and Corn, Analytical Chemistry, 68(18):3187-3193, 1996.
Gea and Turunen, J. Mater. Res., 18(11):2697-2707, 2003.
Gross et al., Chemical Rev., 101(7):2097-2124, 2001.
Hefor and Textor, Langmuir, 17(13): 4014-4020, 2001.
Hehrlein et al., Circulation, 93(4):641-645, 1996.
Helmy and Fadeev, Langmuir, 18:8924-8928, 2002.
Hofer et al., Langmuir, 17(13):4014-4020, 2001.
Hoffman et al., Circulation, 94(6):1247-1254, 1996.
Hutt et al., Langmuir, 13:2740-2748, 1997.
Ishida et al., Langmuir, 18:10496-10499, 2002.
Jost et al., J. Invasive Cardiol., 10(8):477-479, 1998.
Knotek, Surface Science, 101(1-3): 334-340, 1980.
Komatsu et al., Circulation, 98(3):224-233, 1998.
Konstadinidis et al., Langmuir, 8:1307-1317, 1992.
Kornowski et al., J. Am. Coll. Cardiol., 31(1):224-230, 1998.
Laane, J. Amer. Chem. Soc., 89(5):1144-1147, 1967.
Laboda et al., Biochemistry, 27(7):2313-2319, 1988.
Laibinis et al. J. Am. Chem. Soc., 113:7152-7167, 1991.
Laird et al., Circulation, 93(3):529-536, 1996.
Lakovou et al., JAMA, 293(17):2154-2156, 2005.
Lee et al., J. Phys. Chem. B, 108:2648-2653, 2004.
Lee et al., Langmuir, 14:6419-6423, 1998.
Lin et al., Langmuir, 18(14):5473-5478, 2002.
López et al., Analysis, 17(5):267-272, 2004.
Lougerstay-Madec et al. Anticancer Drug Des., 13(8):995-1007, 1998.
Madec-Lougerstay et al., J. Chem. Soc. Perkin Trans., 1:1369, 1999.
Mahapatro et al., Biomacromolecules, 4:544-551, 2003.
Mahapatro et al., Biomacromolecules, 5(1):62-68, 2004b.
Mahapatro et al., Macromolecules, 37(1):35-40, 2004a.
Marcinko and Fadeev, Langmuir, 20:2270-2273, 2004.
McFadden et al., Lancet., 364:1466-1467, 2004.
Meth and Sukenik, Thin Solid Films, 425(1-2):49-58, 2003.
Morice et al., New England J. Medicine, 346(23):1773-1780, 2002.
Moses et al., New England J. Medicine, 349(14):1315-1323, 2003.
Nebeker et al., J. Am. Coll. Cardiol., 47(1):175-181, 2006.
Nguyen-Ho et al., Catheter Cardiovasc. Mien., 56(2):281-288, 2002.
Norde, Adv. Colloid. Interfac., 25:267-340, 1986.
Okamoto, Bulletin Chem. Soc. Japan., 58(11):3393-3394, 1985.
Onyiriuka, J. Appl. Polymer Sci., 47(12):2187-2194, 2003.
Palegrosdemange et al., J. Am. Chem. Soc., 113:12-20, 1991.
Pan et al., Langmuir, 14:3545-3550, 1998.
Pawsey et al., Langmuir, 18(13):5205-5212, 2002.
Pawsey et al., Langmuir, 18(13):5205-5212, 2002.
Picart et al., Advanced Functional Materials, 15(1):83-94, 2005.
Rectenwald et al., Circulation, 102(14):1697-1702, 2000.
Ren et al., Surface Science, 546:64-74, 2003.
Rjeb et al., Condensed Matter, 5(2):169-172, 2004.
Roberts, J. Chem. Soc.—Perkin Transactions, 1(13):1475-1499, 2001.
Ruan et al., Thin Solid Films, 419(1-2):95-104, 2002.
Ryan et al., J. Amer. Chem. Soc., 82:3601-3604, 1960.
Sagiv, J. Am. Chem. Soc., 102:92-98, 1980.
Schlotter et al., Chemical Physics Letters, 132:93-98, 1986.
Schoenfisch and Pemberton, J. Am. Chem. Soc., 120:4502-4513, 1998.
Schreiber, Progress in Surface Science, 65(5-8):151-256, 2000.
Serruys et al., J. Am. Coll. Cardiol., 39(3):393-399, 2002.
Serruys et al., N. Engl. J. Med., 331(8):489-495, 1994.
Shustak et al., Langmuir, 20(18):7499-7506, 2004.
Singh and Shah, Colloids Surf, a-Physicochem. Engineering, Aspects, 77(3):219-224, 1993.
Singh et al., J. Amer. Oil Chemists Soc., 71(12):1405-1409, 1994.
Stefanadis et al., J. Am. Coll. Cardiol., 28(4):1039-1046, 1996.
Tanaka and Yu, Langmuir, 18(3):797-804, 2002.
Tao, J. Amer. Chem. Soc., 115:4350-4358, 1993.
Tomalia and Frechet, J. Polym. Sci., 40(2): 719, 2002.
Tomalia et al., Chem. Int. Ed. Engl., 29:138, 1990.
Tosatti et al., Langmuir, 18(9):3537-3548, 2002.
Turner et al., Langmuir, 12:4411-4416, 1996.
Ulman, Chemical Rev., 96(4):1533-1554, 1996.
van Alsten, Langmuir, 15(22):7605-7614, 1999.
van der Glessen et al., Circulation, 94(7):1690-1697, 1996.
Virmani et al., Circulation, 109(6):701-705, 2004a.
Virmani et al., Coron. Artery Dis., 15(6):313-318, 2004b.
Wagner et al., J. Phys. Chem. B, 1043291-3297, 2000.
Wawrzynczak and Thorpe, FEBS Lett., 207(2):213-216, 1986.
Wistara et al., Cellulose, 6(4):325-348, 1999.
Yan et al., J. Macromol. Sci—Pol. R., C44:175-206, 2004.
Yang et al., J. Biomaterials, 26(30):5991-5998, 2005.
Yoshida et al., J. Dent. Res., 83(6):454-458, 2004.
Zwahlen et al., Langmuir, 18(10):3957-3962, 2002.

1. A medical device comprising one or more surfaces, one or more self-assembled monolayer molecules attached to the one or more surfaces of the medical device, one or more linkers comprising a first functional group and a second functional group, the first functional group attached to a self-assembled monolayer molecule and the second functional group attached to a therapeutic agent.

2. The medical device of claim 1, further defined as comprising more than one self-assembled monolayer molecules forming one or more self-assembled monolayers (SAM) coating a portion or all of the one or more surfaces of the medical device.

3. The medical device of claim 1, wherein the one or more surfaces of the medical device are comprised of one or more of stainless steel, titanium, tantalum, cobalt, chromium, gold, silver, platinum, a polymer, a polymer derivative, a copolymer, a multi-component copolymer, glass, pyrolytic carbon, alumina, zirconia, titania, graphite, and a ceramic.

4. The medical device of claim 1, wherein the one or more surfaces of the medical device are comprised of an alloy of two or more metals selected from the group consisting of stainless steel, titanium, tantalum, cobalt, chromium, gold, silver, and platinum.

5. The medical device of claim 4, wherein the alloy is nitinol.

6. The medical device of claim 3, wherein the one or more surfaces of the medical device are comprised of one or more polymers selected from the group consisting of poly(ethylene glycol), poly(caprolactone), poly(hydroxyethyl methacrylate), poly(lactic acid), poly(ethylene), poly(glycolic acid), poly(styrene), a poly(anhydride), a poly(urethane), a poly (carbamate), a poly(ester), and a derivative thereof.

7. The medical device of claim 3, wherein the one or more polymers is further defined as a terpolymer or a polymer blend.

8. The medical device of claim 3, wherein the material is stainless steel.

9. The medical device of claim 8, wherein the stainless steel is 316L stainless steel.

10. The medical device of claim 1, comprising a SAM attached to one surface of the medical device.

11. The medical device of claim 10, comprising a SAM attached to a portion of the one or more surface of the medical device.

12. The medical device of claim 1, comprising a SAM attached to more than one surface of the medical device.

13. The medical device of claim 1, wherein the one or more self-assembled monolayer molecules are attached to the one or more surfaces via one or more moieties selected from the group consisting of a thiol, a disulfide, a dithioic acid, a dithiocarbamate, a silane, a chlorosilane, a dichlorosilane, a trichlorosilane, an alkoxysilane, a dialkoxysilane, a trialkoxysilane, a hydroxyamic acid, a phosphate, a phosphonic acid, a carboxylic acid, a hydroxamic acid, an alcohol, an amine, a sulfate, a sulfonate, and a sulfinate.

14. The medical device of claim 13, wherein the one or more self-assembled monolayer molecules are attached to the one or more surfaces via a thiol moiety.

15. The medical device of claim 13, wherein the one or more self-assembled monolayer molecules are attached to the one or more surfaces via a silane or silane derivative.

16. The medical device of claim 13, wherein the one or more self-assembled monolayer molecules are attached to the one or more surfaces via a phosphonate or phosphate.

17. The medical device of claim 1, wherein the one or more self-assembled monolayer molecules are comprised of six to thirty-nine carbon atoms.

18. The medical device of claim 17, wherein the one or more self-assembled monolayer molecules are comprised of eight, nine, ten, eleven or twelve carbon atoms.

19. The medical device of claim 1, wherein a polymer or a peptide are attached to the one or more of the self-assembled monolayer molecules.

20. The medical device of claim 19, wherein the polymer is poly(ethylene glycol).

21. The medical device of claim 19, wherein the peptide is a cellular adhesion peptide.

22. The medical device of claim 1, wherein the one or more self-assembled monolayer molecule comprises more than one type of self-assembled monolayer molecule.

23. The medical device of claim 18, wherein a polymer or peptide is attached to the one or more of the self-assembled monolayer molecules, and wherein the one or more self-assembled monolayer molecules are comprised of eight to twelve carbons.

24. The medical device of claim 23, wherein the polymer is poly(ethylene glycol).

25. The medical device of claim 23, wherein the peptide is a cellular adhesion peptide.

26. The medical device of claim 22, wherein the one or more self-assembled monolayer molecules are attached to the one or more surfaces via one or more moieties selected from the group consisting of a thiol, a disulfide, a dithioic acid, a dithiocarbamate, a silane, a chlorosilane, a dichlorosilane, a trichlorosilane, an alkoxysilane, a dialkoxysilane, a trialkoxysilane, a hydroxyamic acid, a phosphate, a phosphonic acid, a carboxylic acid, a hydroxamic acid, an alcohol, an amine, a sulfate, a sulfonate, and a sulfinate.

27. The medical device of claim 1, wherein the one or more linkers are selected from the group consisting of a sulfate, polyethylene glycol, a dendrimer, a molecule comprising a tert-butyl protecting group, a molecule comprising an isobutylene oxide connection, an amino benzyl alcohol, a hydroxy benzyl alcohol connection, an aminobenzene dimethanol, an aminobenzene trimethanol, a hydroxybenzene dimethanol, a hydroxybenzene trimethanol, a vinyl sulfoxide, a substituted vinyl sulfoxide, a substituted methoxymethyl connection, a substituted vinyl ether connection, a carbonate connection, an ester connection, an anhydride connection, a substituted carbamic anhydride connection, a carbonic anhydride connection, an substituted urea connection, a substituted urethane connection, a substituted guanidine connection, a ether connection, a mercaptan connection, a sulfoxide connection, a sulfinate connection, a sulfonate connection, a sulfenate connection, a nitronate connection, a sulfite connection, a sulfate connection, a phosphate connection, a phosphonate connection, a phosphine connection, a silane connection, a silicate connection, a disulfide connection, a peroxide connection, an alkane connection, an alkene connection, an alkyne connection, an iodonium connection, an amino connection, a substituted allyl ether connection, a substituted benzyl ether connection and an imine connection.

28. The medical device of claim 1, where the one or more linkers are further defined as a dendritic structure or dendrimer.

29. The medical device of claim 28, wherein the dendritic structure or dendrimer is further defined as being capable of disassembly, self-immolation, release by dendritic amplification, or cascade-release.

30. The medical device of claim 1, wherein the first and second functional groups of the one or more linkers are selected from the group consisting of a hydroxyl, a carboxyl, an amino, a phosphate, a phosphonate, a sulfate, a sulfite, a sulfenate, a sulfinate, a sulfonate, a sulfoxide, a sulfone, an amide, an ester, an ketone, an aldehyde, a nitrile, an alkene, an alkyne, an ether, a thiol, a hydroxyamic acid, a silane, a silicate, a carbamodithionate, a dithionate, a mercaptan, a disulfide, a peroxide and a nitronate.

31. The medical device of claim 1, wherein at least one self-assembled monolayer molecule is covalently bound to the first functional group of the one or more linkers.

32. The medical device of claim 1, wherein at least one self-assembled monolayer molecule is non-covalently bound to the first functional group of the one or more linkers.

33. The medical device of claim 1, wherein the second functional group of the one or more linkers is covalently bound to the therapeutic agent.

34. The medical device of claim 1, wherein the second functional group of the one or more linkers is non-covalently bound to the therapeutic agent.

35. The medical device of claim 1, wherein the therapeutic agent is selected from the group consisting of a small molecule, a peptide, a polypeptide, a protein, an enzyme, an antibody, a DNA molecule, and an RNA molecule.

36. The medical device of claim 1, wherein the therapeutic agent is an anticancer agent, a hormone, an anesthetic agent, a vasodilator, an anticoagulant, an anti-inflammatory agent, a steroid, an antibiotic, an antiseptic, an antifungal, an opiate, an analgesic, an antiproliferative agent, or an anti-platelet agent.

37. The medical device of claim 36, wherein the therapeutic agent is rapamycin, sirolimus, a taxol, everolimus, tacrolimus, dexamethasone, prednisolone, morphine, or fentanyl.

38. The medical device of claim 37, wherein the taxol is paclitaxel.

39. The medical device of claim 1, further defined as comprising more than one type of therapeutic agent attached to the one or more linkers.

40. The medical device of claim 1, wherein the medical device is further defined as a medical device suitable for implantation in a subject.

41. The medical device of claim 1, wherein the medical device is selected from the group consisting of a stent, a valve, a metal plate, a musculoskeletal fixation system, a pin, an artificial joint, a dental implant, a temporal mandibular joint, an ocular implant, a neural implant, an artificial heart, and an artificial organ, and an implant in contact with body fluids.

42. The medical device of claim 41, wherein the stent is selected from the group consisting of a coronary stent, an arterial stent, a GI stent, a pulmonary stent, a vascular stent, and a ureteral stent.

43. The medical device of claim 1, wherein the medical device is further defined as a medical device suitable for application to a surface of a subject.

44. The medical device of claim 43, wherein the surface of the subject is a skin surface, a mucosal surface, a wound surface, a surface of a hollow viscus, or a tumor surface.

45. The medical device of claim 1, wherein the medical device comprises one or more openings in one or more surfaces of the medical device.

46. The medical device of claim 1, wherein the medical device is further defined as a medical device with one or more nanoporous surfaces.

47. The medical device of claim 46, wherein the medical device is further defined as a medical device with a nanoporous body.

48. The medical device of claim 45, wherein the one or more self-assembled monolayer molecules are attached to the surface of the medical device that comprises the one or more openings.

49. The medical device of claim 1, further defined as a medical device capable of releasing the therapeutic agent in a subject following contact of the medical device with a subject.

50. The medical device of claim 49, wherein the therapeutic agent is released by hydrolysis, oxidation, reduction, cycloaddition, retro-cycloaddition, ring-closure, decomposition, disproportionation, electrophilic cleavage, nucleophilic cleavage, aminolysis, alcoholysis, elimination, and solvolysis, acid catalysis, biocatalysis, or base catalysis following implantation of the medical device in a subject.

51-121. (canceled)

122. A method of administering a therapeutic agent to a subject, comprising:
(a) obtaining a medical device comprising one or more surfaces, one or more self-assembled monolayer molecules attached to the one or more surfaces of the medical device, one or more linkers comprising a first functional group and a second functional group, the first functional group attached to a self-assembled monolayer molecule and the second functional group attached to a therapeutic agent; and
contacting the subject with the medical device.

123. The method of claim 122, wherein the medical device is further defined as comprising more than one self-assembled monolayer molecule forming a self-assembled monolayer (SAM) on one or more surface of the medical device.

124. The method of claim 122, further comprising release of the therapeutic agent following contact of the medical device with the subject.

125. The method of claim 122, wherein the subject is a mammal.

126. The method of claim 125, wherein the mammal is a human.

127. The method of claim 126, wherein the human is a patient in need of the therapeutic agent or treatment or prevention of a disease.

128. The method of claim 127, wherein the disease is selected from the group consisting of cardiovascular disease, hyperproliferative disease, coronary artery disease, valvular heart disease, heart failure, peripheral vascular disease, ureteral obstruction, bile duct obstruction, bronchial or tracheal obstruction, arthritis, degenerative joint disease, a bone fracture, arthritis, degenerative joint disease, cancer, or a cardiac arrhymthia.

129. The method of claim 128, wherein the patient is further defined as a patient in need of surgical therapy with implantation or application of a medical device for treatment or prevention of cardiovascular disease, hyperproliferative disease, coronary artery disease, valvular heart disease, heart failure, peripheral vascular disease, ureteral obstruction, bile duct obstruction, bronchial or tracheal obstruction, arthritis, degenerative joint disease, a bone fracture, arthritis, degenerative joint disease, cancer, or a cardiac arrhymthia.

130. The method of claim 122, wherein the therapeutic agent is selected from the group consisting of a small molecule, a peptide, a polypeptide, a protein, an enzyme, an antibody, a DNA molecule, and an RNA molecule.

131. The method of claim 122, wherein the therapeutic agent is an anticancer agent, a hormone, an anesthetic agent, a vasodilator, an anticoagulant, an anti-inflammatory agent, a steroid, an antibiotic, an antiseptic, an antifungal, an opiate, an analgesic, an antiproliferative agent, and an anti-platelet drug.

132. The method of claim 122, wherein the therapeutic agent is rapamycin, sirolimus, a taxol, everolimus, tacrolimus, dexmethasone, prednisolone, morphine, or fentanyl.

133. The method of claim 132, wherein the taxol is paclitaxel.

134. The method of claim 122, wherein the medical device comprises one or more material selected from the group consisting of stainless steel, titanium, tantalum, cobalt, chromium, gold, silver, platinum, a polymer, a polymer derivative, a copolymer, a multi-component copolymer, glass, pyrolytic carbon, alumina, zirconia, titania, graphite, and a ceramic.

135. The method of claim 134, wherein the one or more surface of the medical device is comprised of an alloy of two or more metal selected from the group consisting of stainless steel, titanium, tantalum, cobalt, chromium, gold, silver, and platinum.

136. The method of claim 135, wherein the alloy is nitinol.

137. The method of claim 134, wherein the medical device is comprised of one or more polymer selected from the group consisting of poly(ethylene glycol), poly(caprolactone), poly(hydroxyethyl methacrylate), poly(lactic acid), poly(ethylene), poly(glycolic acid), poly(styrene), a poly(anhydride), a poly(urethane), a poly (carbamate), a poly(ester), and a derivative thereof.

138. The of claim 134, wherein the multi-component polymer is further defined as a terpolymer or a polymer blend.

139. The method of claim 134, wherein the material is stainless steel.

140. The method of claim 139, wherein the stainless steel is 316L stainless steel.

141. The method of claim 122, wherein the SAM is attached to one surface of the medical device.

142. The method of claim 122, wherein the SAM is attached to a portion of the one or more surfaces of the medical device.

143. The method of claim 122, wherein the SAM is attached to a single surface of the medical device.

144. The method of claim 122, wherein the one or more self-assembled monolayer molecules are attached to the one or more surfaces via one or more moieties selected from the group consisting of a thiol, a disulfide, a dithioic acid, a dithiocarbamate, a silane, a chlorosilane, a dichlorosilane, a trichlorosilane, an alkoxysilane, a dialkoxysilane, a trialkoxysilane, a hydroxyamic acid, a phosphate, a phosphonic acid, a carboxylic acid, a hydroxamic acid, an alcohol, an amine, a sulfate, a sulfonate, and a sulfinate.

145. The method of claim 122, wherein the one or more self-assembled monolayer molecules comprise more than one type of self-assembled monolayer molecule.

146. The method of claim 122, wherein a polymer or peptide is attached to the one or more self-assembled monolayer molecules, and the one or more self-assembled monolayer molecules are comprised of eight to twelve carbons.

147. The method of claim 122, comprising more than one self-assembled monolayer (SAM) on the one or more surfaces of the medical device.

148. The method of claim 122, wherein one or more linkers are selected from the group consisting of a sulfate, polyethylene glycol, a dendrimer, a molecule comprising a tert-butyl protecting group, a molecule comprising an isobutylene oxide connection, an amino benzyl alcohol, a hydroxy benzyl alcohol connection, an aminobenzene dimethanol, an aminobenzene trimethanol, a hydroxybenzene dimethanol, a hydroxybenzene trimethanol, a vinyl sulfoxide, a substituted vinyl sulfoxide, a substituted methoxymethyl connection, a substituted vinyl ether connection, a carbonate connection, an ester connection, an anhydride connection, a substituted carbamic anhydride connection, a carbonic anhydride connection, an substituted urea connection, a substituted urethane connection, a substituted guanidine connection, a ether connection, a mercaptan connection, a sulfoxide connection, a sulfinate connection, a sulfonate connection, a sulfenate connection, a nitronate connection, a sulfite connection, a sulfate connection, a phosphate connection, a phosphonate connection, a phosphine connection, a silane connection, a silicate connection, a disulfide connection, a peroxide connection, an alkane connection, an alkene connection, an alkyne connection, an iodonium connection, an amino connection, a substituted allyl ether connection, a substituted benzyl ether connection and an imine connection.

149. The method of claim 122, wherein the first and second functional group of the linkers are selected from the group consisting of a hydroxyl, a carboxyl, an amino, a phosphate, a phosphonate, a sulfate, a sulfite, a sulfenate, a sulfinate, a sulfonate, a sulfoxide, a sulfone, an amide, an ester, an ketone, an aldehyde, a nitrile, an alkene, an alkyne, an ether, a thiol, a hydroxyamic acid, a silane, a silicate, a carbamodithionate, a dithionate, a mercaptan, a disulfide, a peroxide and a nitronate.

150. The method of claim 122, wherein at least one self-assembled monolayer molecule is covalently bound to the first functional group of the linker.

151. The method of claim 122, wherein at least one self-assembled monolayer molecule is non-covalently bound to the first functional group of the linker.

152. The method of claim 122, wherein the second functional group of the linker is covalently bound to the therapeutic agent.

153. The method of claim 122, wherein the second functional group of the linker is non-covalently bound to the therapeutic agent.

154. The method of claim 122, wherein the medical device is selected from the group consisting of a stent, a valve, a metal plate, a musculoskeletal fixation system, a pin, an artificial joint, a dental implant, a temporal mandibular joint, an ocular implant, a neural implant, an artificial heart, and an artificial organ, or an implant in contact with body fluids.

155. The method of claim 154, wherein the stent is a coronary stent, an arterial stent, a GI stent, a pulmonary stent, a vascular stent, a ureteral stent.

156. The method of claim 122, wherein the medical device is further defined as a medical device suitable for application to a surface of the subject.

157. The method of claim 156, wherein the surface is a skin surface, a mucosal surface, a wound surface, a surface of a hollow viscus, or a tumor surface.

158. The method of claim 122, further defined as a medical device comprising one or more openings in one or more surfaces of the medical device.

159. The method of claim 122, wherein the self-assembled monolayer molecules are attached to the one or more surfaces of the medical device that comprise the one or more openings.

160. The method of claim 122, further defined as a medical device capable of releasing the therapeutic agent in a subject following contact of the medical device with a subject.

161. The method of claim 160, wherein the therapeutic agent is released by hydrolysis, hydrolysis, oxidation, reduction, cycloaddition, retro-cycloaddition, ring-closure, decomposition, disproportionation, electrophilic cleavage, nucleophilic cleavage, aminolysis, alcoholysis, elimination, and solvolysis, acid catalysis, or base catalysis following implantation of the medical device in a subject.

162. The method of claim 122, further comprising identifying a subject in need of the therapeutic agent.

163. The method of claim 162, further comprising identifying a subject in need of the medical device.

164. The method of claim 122, further defined as a method of preventing a disease in a subject.

165. The method of claim 164, further comprising identifying a patient in need of preventive therapy.

166. The method of claim 122, further comprising administration of one or more secondary forms of therapy.

167. The method of claim 166, wherein the one or more secondary forms of therapy are selected from the group consisting of pharmacotherapy, surgical therapy, radiation therapy, chemotherapy, gene therapy, and immunotherapy.

168. The medical device of claim 27, wherein the linker is a sulfate.

169. The method of claim 148, wherein the linker is a sulfate.
